(19) United States
(12) Patent Application Publication
(10) Pub. No.: US 2003/0083357 A1
(43) Pub. Date: May 1, 2003

(54) Oxime derivatives for the treatment of dyslipidemia and hypercholesteremia

(76) Inventors: Magnus Pfahl, Solana Beach, CA (US); Catherine Tachdjian, San Diego, CA (US); Hussien A. Al-Shamma, Encinitas, CA (US)

Correspondence Address: NEEDLE & ROSENBERG P C, 127 PEACHTREE STREET N E, ATLANTA, GA, 30303-1811, US

(21) Appl. No.: 10/224,288

(22) Filed: Aug. 19, 2002

Related U.S. Application Data
(60) Provisional application No. 60/313,199, filed on Aug. 17, 2001.

(51) Int. Cl. C07D 213/54; A61K 31/44; A61K 31/40; A61K 31/15; A61K 31/165

(52) U.S. Cl. 514/357; 514/428; 514/640; 514/534; 514/619; 546/329; 548/566; 560/35; 564/256; 564/163

(57) ABSTRACT

The present invention relates to compounds of Formula (I) which may be useful in the treatment of diseases, such as, metabolic disorders, dyslipidemia and/or hyperchloesterolemia:

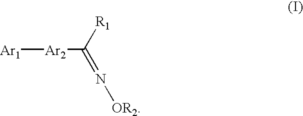

### RELATED APPLICATIONS

This application claims priority to the U.S. Provisional Application Serial No. 60/313,199, filed Aug. 17, 2001, the disclosure of which application is hereby incorporated in its entirety by this reference.

### BACKGROUND OF THE INVENTION

Western diets that are rich in fats combined with sedentary lifestyles have led to increased risks for the development of a number of metabolic disorders including dyslipidemia, hypercholesteremia and obesity. Obesity has reached epidemic proportions in the U.S. and other developed countries and even in several developing countries. While approximately 10% of the obese population eventually develops Type 2 diabetes, a significantly larger percentage of obese persons have elevated levels of blood lipids, including elevated fatty acids, triglycerides and cholesterol. These dyslipidemic or hypercholesteremic patients are at risk for a number of diseases including artherosclerosis and heart disease. Well balanced diets, restricted calorie intake and exercise have been well accepted as effective approaches to the reversal/treatment of these disorders. At the same time, it has become apparent that these simple “natural” regiments cannot be successfully employed or managed by a majority of the obese population. Drugs that are safe and effective in controlling dietary or genetically based dyslipidemia or hypercholesteremia are therefore the only alternative solution to prevent and counteract serious disease development or progression in the affected patients. While anti-dyslipidemic drugs (for instance the fibrates) and cholesterol lowering drugs have been developed, they either do not have optimal safety profiles, or lack in efficacy for the control of dietary induced and dependant or genetically based dyslipidemia and hypercholesteremia. Thus, there is a need in the art for new drugs for the treatment of diseases such as dyslipidemia and hypercholesteremia.

### SUMMARY OF THE INVENTION

In accordance with the purposes of this invention, as embodied and broadly described herein, this invention, in one aspect, relates to compositions and methods related to metabolism.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 shows the total cholesterol levels in HSD Rats maintained on an atherogenic diet after treatment with Compound 1.

FIG. 2 shows the LDL cholesterol levels in HSD Rats maintained on an atherogenic diet after treatment with Compound 1.

FIG. 3 shows the improvement of glucose tolerance in Zuker Fatty Rats treated for one week with Compound 1.

FIG. 4 shows representative examples of methods for the synthesis of compounds disclosed herein.

### DETAILED DESCRIPTION

Definitions

In the specification and Formulae described herein the following terms are hereby defined.

The term “alkyl” denotes a saturated hydrocarbon radical. Alkyl radicals may be branched or unbranched, and are structurally similar to a non-cyclic alkane compound modified by the removal of one hydrogen from the non-cyclic alkane and the substitution therefore of a non-hydrogen group or residue. Alkyls comprise a noncyclic, saturated, straight or branched chain hydrocarbon residue having from 1 to 12 carbons, 1 to 9 carbons, 1 to 8 carbons, or 1 to 6 carbons. Lower alkyl radicals have 1 to 4 carbon atoms. Examples of alkyl and lower alkyl radicals include but are not limited to methyl, ethyl, n-propyl, iso-propyl, n-butyl, sec-butyl, t-butyl, amyl, t-amyl, n-pentyl, n-hexyl, i-octyl, n-nonyl and like radicals.

The term “alkenyl” denotes an unsaturated hydrocarbon radical containing at least one carbon-carbon double bond. Alkenyl radicals are structurally similar to a non-cyclic alkene compound modified by the removal of one hydrogen from the non-cyclic alkene and the substitution therefore of a non-hydrogen group or residue. Alkenyl radicals may have 1 to 12 carbons, 1 to 9 carbons, 1 to 8 carbons, or 1 to 6 carbons. Lower alkenyl radicals have 1 to 4 carbon atoms. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

The term “alkynyl” denotes a hydrocarbon radical containing at least one triple bond. Alkynyl radicals may have 1 to 12 carbons, 1 to 9 carbons, 1 to 8 carbons, or 1 to 6 carbons. Lower alkynyl radicals have 1 to 4 carbon atoms. Examples include but are not limited to ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “substituted alkyl” denotes an alkyl radical bonded to one or more organic or inorganic substituent radicals. Substituted alkyls are an alkyl radical as referenced in the above definition that is further substituted with one, two, or more additional organic or inorganic substitutuent groups. Suitable organic and inorganic substituents include but are not limited to hydroxyl, halogen, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. When the alkyl is substituted with more than one group then they may be the same or different.

The term “substituted alkenyl” denotes an alkenyl radical bonded to one or more organic or inorganic substituent radicals, or preferably one, two, or more such substitutents. Suitable organic and inorganic substituents include but are not limited to halogen, hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. When the alkenyl is substituted with more than one group then they may be the same or different.

The term “substituted alkynyl” denotes an alkynyl radical containing 1 to 9 carbons bonded to one or more organic or inorganic substituent radicals. Suitable organic and inorganic substituents include but are not limited to halogen, hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. When the alkynyl is substituted with more than one group then they may be the same or different.

The term “cycloalkyl” denotes a carbocyclic radical which is structurally similar to a cyclic alkane compound modified by the removal of at least one hydrogen from the cyclic alkane and substitution therefore of a non-hydrogen group or residue. Cycloalkyl groups, or residues radical may contain 1 to 8 ring carbons, 2 to 7 ring carbons, 3 to 6 ring carbons, 4 to 5 ring carbons, 3 to 18 ring carbons, 4 to 12 ring carbons, or 5 to 8 ring carbons. A cycloalkyl radical may refer to an exocyclic radical fused to an aryl or heteroaryl ring, in which case the number of carbon atoms excludes the aromatic carbon atoms that are part of the aryl or heteroaryl ring. containing 3 to 8 ring carbons. Examples include but are not limited to cyclopropyl, cyclobutyl, cyclopentyl, cyclopentyl, cyclohexyl, cycloheptyl and the like.

The term “substituted cycloalkyl” denotes a cycloalkyl as defined above bonded to one or more organic or inorganic substituent radicals. Suitable organic and inorganic substituents include but are not limited to halogen, alkyl, hydroxyl, alkoxy, substituted alkoxy, carboxy, carboalkoxy, alkylcarboxamide, substituted alkylcarboxamide, dialkylcarboxamide, substituted dialkylcarboxamide, amino, mono-substituted amino or di-substituted amino. When the cycloalkyl is substituted with more than one group, they may be the same or different.

The term “cycloalkenyl” denotes a cycloalkyl radical as defined above additionally having at least one double bond in the ring. If the cycloalkenyl ring is fused to an aryl or heteroaryl ring, the double bond is in addition to the “double” bond of that is integral to the aryl or heteroaryl ring. Examples include but are not limited to cyclopropenyl, 1-cyclobutenyl, 2-cyclobutenyl, 1 -cyclopentenyl, 2-cyclopentenyl, 3-cyclopentenyl, 1-cyclohexyl, 2-cyclohexyl, 3-cyclohexyl and the like.

The term “substituted cycloalkenyl” denotes a cycloalkenyl radical bonded to one or more organic or inorganic substituent radicals. Suitable organic and inorganic substituents include but are not limited to halogen, alkyl, hydroxyl, alkoxy, substituted alkoxy, haloalkoxy, carboxy, carboalkoxy, alkylcarboxamide, substituted alkylcarboxamide, dialkylcarboxamide, substituted dialkylcarboxamide, amino, mono-substituted amino or di-substituted amino. When the cycloalkenyl is substituted with more than one group, they may be the same or different.

The term “alkoxy” as used herein denotes a substituent radical comprising an oxygen atom with an alkyl radical bound thereto. Examples include but are not limited to methoxy, ethoxy, n-propoxy, iso-propoxy, n-butoxy, t-butoxy, iso-butoxy and the like.

The term “substituted alkoxy” denotes an alkoxy radical as defined above bonded to one or more organic or inorganic substituent radicals. Suitable organic and inorganic substituents include but are not limited to hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. When the alkoxy is substituted with more than one group is present then they may be the same or different.

The term amino denotes a substituted or unsubstituted trivalent nitrogen-containing radical or group that is structurally related to ammonia (NH₃) by the substitution of one or more of the hydrogen atoms of ammonia by a substitutent group or radical.

The term “mono-substituted amino” denotes an amino substituted with one radicals selected from alkyl, substituted alkyl or arylalkyl wherein the terms have the same definitions found herein.

The term “di-substituted amino” denotes an amino substituted with two radicals that may be same or different selected from aryl, substituted aryl, alkyl, substituted alkyl or arylalkyl wherein the terms have the same definitions as disclosed herein. Examples include but are not limited to dimethylamino, methylethylamino, diethylamino and the like.

The term “haloalkyl” denotes an alkyl radical, as defined above, substituted with one or more halogens, such as fluorine, chlorine, bromine, or iodine preferably fluorine. Examples include trifluoromethyl, pentafluoroethyl and the like.

The term “haloalkoxy” denotes a haloalkyl, as defined above, that is directly attached to an oxygen to form trifluoromethoxy, pentafluoroethoxy and the like.

The term “acyl” denotes a radical containing a carbonyl (—C(O)—R group) wherein the R group is hydrogen or has 1 to 8 carbons, such as, for example, formyl, acetyl, propionyl, butanoyl, iso-butanoyl, pentanoyl, hexanoyl, heptanoyl, benzoyl and the like.

The term “acyloxy” denotes a radical containing a carboxyl (—O—C(O)—R) group wherein the R group comprises hydrogen or 1 to 8 carbons. Examples include but are not limited to acetyloxy, propionyloxy, butanoyloxy, iso-butanoyloxy, benzoyloxy and the like.

The term “aryl” denotes a radical comprising at least one unsaturated and conjugated six membered ring analogous to the six membered ring of benzene. Aryl radicals having such unsaturated and conjugated rings are also known to those of skill in the art as “aromatic” radicals. Preferred aryl radicals have 6 to 12 ring carbons. Aryl radicals include but are not limited to aromatic radicals comprising phenyl and naphthyl ring radicals.

The term “substituted aryl” denotes an aromatic radical whose aromatic ring is bonded to one or more organic or inorganic substituent groups, radicals, or residues. Suitable organic and inorganic substituents for aryl radicals include but are not limited to hydroxyl, cycloalkyl, aryl, substituted aryl, heteroaryl, heterocyclic ring, substituted heterocyclic ring, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, alkoxy, substituted alkoxy or haloalkoxy radicals, wherein the terms are defined herein. When an aryl radical is substituted with more than one substituent group radical, or residue then they may be the same or different.

The term “halo” or “halogen” refers to a fluoro, chloro, bromo or iodo group.

The term “alkylsulfonyl” refers to a sulfone radical containing 1 to 8 carbons, linear or branched. Examples include but are not limited to methylsulfonyl, ethylsulfonyl, isopropylsulfonyl having the structures CH₃S(O)₂—, CH₃CH₂S(O)₂—, (CH₃)₂CHS(O)₂— respectively and the like.

The term “alkylsulfinyl” refers to a sulfoxide radical containing 1 to 8 carbons, linear or branched. Examples include but are not limited to methylsulfinyl, ethylsulfinyl, isopropylsulfinyl having the structures CH3S(O)—, CH₃CH₂S(O)—, (CH₃)₂CHS(O)— respectively and the like.

The term “thioalkyl” refers to a sulfide radical containing 1 to 8 carbons, linear or branched. Examples include but are not limited to methylsulfide, ethyl sulfide, isopropylsulfide having the structures CH₃S—, CH₃CH₂S—, (CH₃)₂CHS— respectively and the like.

The term “thiohaloalkyl” denotes a thioalkyl radical wherein the alkyl moiety is substituted with one or more halogens. Examples include but are not limited to trifluoromethylthio, 1,1-difluoroethylthio, 2,2,2-trifluoroethylthio and the like.

The term “carboalkoxy” refers to an alkyl ester of a carboxylic acid, wherein alkyl has the same definition as found above. Examples include but are not limited to carbomethoxy, carboethoxy, carboisopropoxy and the like.

The term “alkylcarboxamide” denotes a radical having the structure —N(R)—C(O)— or —C(O)—N(R)— wherein a single alkyl group R is attached to the nitrogen atom of an amide, i.e.. Examples include but are not limited to N-methylcarboxamide, N-ethylcarboxamide, N-(iso-propyl)carboxamide and the like.

The term “substituted alkylcarboxamide” denotes a residue having “substituted alkyl” group attached to the nitrogen atom of an alkylcarboxamide residue.

The term “dialkylcarboxamide” denotes two alkyl or arylalkyl R groups that are the same or different attached to the nitrogen atom of a carboxamide (—C(O)—N(R′)(R″)) radical. Examples include but are not limited to N,N-dimethylcarboxamide, N-methyl-N-ethylcarboxamide and the like.

The term “substituted dialkylcarboxamide” denotes dialkylcarboxamide residue having two alkyl groups attached to the nitrogen of the dialkylcarboxyamide residue, where one or both groups is a “substituted alkyl”, as defined above. It is understood that these groups may be the same or different. Examples include but are not limited to N,N-dibenzylcarboxamide, N-benzyl-N-methylcarboxamide and the like.

The term “alkylamide” denotes a residue comprising an acyl radical attached to the nitrogen of an amine or monoalkylamine residue, wherein the term acyl has the same definition as found above. Examples of an alkylamide include but are not limited to acetamido, propionamido and the like.

The term “alkylene” denotes an acyclic or cyclic hydrocarbyl radical containing one to nine carbons that bridges two groups, such as, for example, Ar₁ and Ar₂, to give Ar₁-alkylene-Ar₂. Examples of alkylene radicals include but are not limited to:

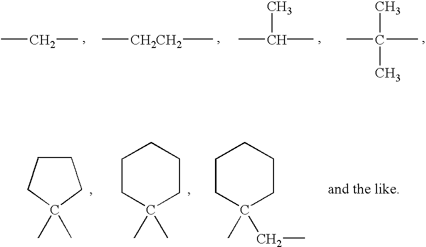

The term “substituted alkylene” denotes an alkylene radical defined above containing one to nine carbons that is further substituted with at least one additional group, selected from but not limited to hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. When the alkylene is substituted with more than one group then they may be the same or different.

The term “heterocyclic ring” is a radical that comprises at least a five-membered or six-membered ring that are completely or partially saturated and comprise at least one ring heteroatom but no more than three ring heteroatoms, selected from nitrogen, oxygen and/or sulfur. Examples include but are not limited to morpholino, piperidinyl, piperazinyl, tetrahydrofuranyl and the like.

The term “substituted heterocyclic ring” refers to a heterocyclic ring bonded to one or more organic or inorganic substituent radicals. Suitable organic and inorganic substituents include but are not limited to halogen, hydroxyl, alkyl, substituted alkyl, haloalkyl, phenyl, substituted phenyl, heteroaryl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, alkoxy, substituted alkoxy or haloalkoxy. When the heterocyclic ring is substituted with more than one group then the groups may be the same or different.

The term “heteroaryl” is an aromatic radical that comprises at least a five-membered or six-membered unsaturated and conjugated ring containing at least two ring carbon atoms and 1 to 4 ring heteroatoms selected from nitrogen, oxygen and/or sulfur. Such heteroaryl radicals are often alternatively termed “heteroaromatic” by those of skill in the art. In some embodiments the heteroaryl radicals have from two to twelve ring carbon atoms, or alternatively 4 to 5 ring carbon atoms in the heteroaryl ring. Examples include, but are not limited to, pyridinyl, pyrimidinyl, pyrazinyl, pyrrolyl, furanyl, tetrazolyl, isoxazolyl, oxadiazolyl, benzothiophenyl, benzofuranyl, quinolinyl, isoquinolinyl and like radicals or residues.

The term “substituted heteroaryl” denotes a heteroaryl radical as defined above wherein the heteroaryl ring is bonded to one or more organic or inorganic substituent radicals. Suitable organic and inorganic substituent radicals for heteroaryl radicals include but are not limited to hydroxyl, cycloalkyl, aryl, substituted aryl, heteroaryl, heterocyclic ring, substituted heterocyclic ring, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, alkoxy, substituted alkoxy or haloalkoxy radicals, wherein the terms are defined herein. When the heteroaryl is substituted with more than one substituent group then they may be the same or different. The organic substituent radicals may each comprise between 1 and 18 carbon atoms, 1 and 12 carbon atoms, 1 and 6 carbon atoms, or between 1 and 4 carbon atoms.

The term “amide” as defined hereby and used in the instant specification refers to a functional group or residue that contains a carbonyl (CO) group bound to a nitrogen atom, i.e. a residue having the formula:

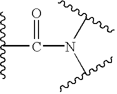

The term radical, as used in the specification and concluding claims, refers to a fragment, group, or substructure of an organic compound regardless of how the compound is prepared, or the presence of other substituent groups on the radical. For example, certain embodiments of the invention comprise 5,6,7,8-tetrahydro-2-naphthyl radicals, i.e. fragments having the structure:

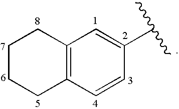

A 5,6,7,8-tetrahydro-2-naphthyl radical itself further comprises a benzene radical and a cyclohexene radical, or may be further substituted with one or more other substitutent groups or radicals, including, for example, methyl or other radicals, as disclosed elsewhere herein. The term “radical” as used herein is not to be confused with certain reactive chemical compounds having unpaired electrons, known to those of skill in the art as “free radicals.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH2CH2O— repeat units in the polyester, regardless of whether ethylene glycol is used to prepare the polyester.

By the term “effective amount” of a compound or composition as provided herein is meant a nontoxic but sufficient amount of the compound or composition to provide the desired function, such as the inhibition or activation of a particular enzyme, a regulation of gene expression, or the treatment of a disease condition. The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount may be determined by one of ordinary skill in the art using only routine experimentation.

It must be noted that, as used in the specification and the appended claims, the singular forms “a,” “an” and “the” can include plural referents unless the context clearly dictates otherwise. Thus, for example, reference to “an aromatic compound” includes mixtures of aromatic compounds.

Compositions

Some disclosed embodiments of the invention relate to compounds of Formula (I):

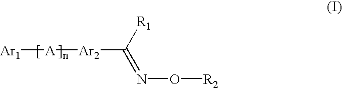

wherein n is 0 or 1;

Ar₁ is a substituted or unsubstituted aryl radical or a substituted or unsubstituted heteroaryl radical;

Ar₂ is a substituted or unsubstituted aryl radical or a substituted or unsubstituted heteroaryl radical;

A is a substituted or unsubstituted bridging radical comprising a connected chain of atoms comprising from one to nine carbon atoms and optionally comprising one or two heteroatoms selected from O, S and N, wherein N is further substituted with hydrogen, alkyl or substituted alkyl;

R₁ is hydrogen, a substituted or unsubstituted amino radical, or a substituted or unsubstituted organic radical comprising from one to 12 carbon atoms; and

R₂ is hydrogen, or a substituted or unsubstituted organic radical comprising from one to 12 carbon atoms;

or a pharmaceutically acceptable salt thereof.

In some embodiments, the Ar₁ radicals may comprise an aryl or heteroaryl radical optionally substituted with hydrogen, alkyl, substituted alkyl, alkenyl, substituted alkenyl, alkynyl, substituted alkynyl, halogen, cyano, nitro, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, alkylsulfonamide, arylsulfonamide, alkylurea, arylurea, alkylcarbamate, arylcarbamate, heteroaryl, haloalkoxy, alkylsulfonyl, alkylsulfinyl, thioalkyl or thiohaloalkyl radicals.

In other embodiments, the Ar₁ radical may comprise a substituted aryl or heteroaryl radical wherein two substituents together with the aryl or heteroaryl ring of Ar₁ form a cycloalkyl, substituted cycloalkyl, cycloalkenyl or substituted cycloalkenyl optionally comprising 1 or 2 heteroatoms selected from O, S, SO, SO₂ and N, wherein N is further substituted with hydrogen, alkyl or substituted alkyl. In these embodiments, Ar₁ may be optionally substituted with hydrogen, alkyl, substituted alkyl, alkenyl, substituted alkenyl, alkynyl, substituted alkynyl, halogen, cyano, nitro, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, alkylsulfonamide, arylsulfonamide, alkylurea, arylurea, alkylcarbamate, arylcarbamate, heteroaryl, haloalkoxy, alkylsulfonyl, alkylsulfinyl, thioalkyl or thiohaloalkyl radicals. The organic substituent radicals may each independently comprise between 1 and 18 carbon atoms, 1 and 12 carbon atoms, 1 and 6 carbon atoms, or between 1 and 4 carbon atoms.

Certain embodiments of the invention relate to compounds wherein n is 0 or 1; i.e. the bridging “A” group may be either present or absent, so as to give compounds of the structures indicated below.

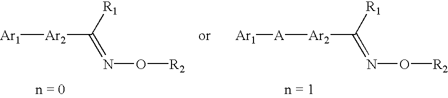

The bridging A groups may comprise an alkylene or substituted alkylene group or radical optionally comprising 1 or 2 heteroatoms selected from O, S and N, wherein the heteroatoms are substituted for a carbon atom of an A group. N atoms may be further substituted with a variety of substituent groups, including hydrogen, alkyl or substituted alkyl.

Examples of bridging “A” radicals having heteroatoms therein include, for example:

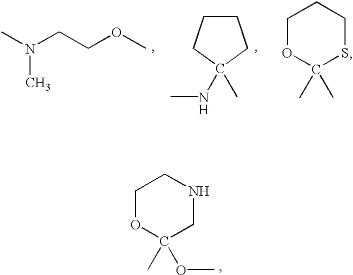

In some embodiments, the Ar₂ radical of the compounds of the invention may be an aryl or heteroaryl optionally substituted with hydrogen, alkyl, substituted alkyl, alkenyl, substituted alkenyl, alkynyl, substituted alkynyl, halogen, cyano, nitro, hydroxyl, acyloxy, alkoxy, substituted alkoxy, hydroxyl, acyl, amino, mono-substituted amino, di-substituted amino, carboxy, carboalkoxy, alkylcarboxamide, substituted alkylcarboxamide, dialkylcarboxamide, substituted dialkylcarboxamide, alkylsulfonamide, arylsulfonamide, alkylurea, arylurea, alkylcarbamate, arylcarbamate, heteroaryl, alkoxy, substituted alkoxy, haloalkoxy, alkylsulfonyl, alkylsulfinyl, thioalkyl or thiohaloalkyl.

In some embodiments, the R₁ radical may be hydrogen, or a substituted or unsubstituted organic radical comprising from one to 12 carbon atoms, from one to 6 carbon atoms, or from one to four carbon atoms. Suitable radicals include but are not limited to an alkyl, substituted alkyl, cycloalkyl, substituted cycloalkyl, amino, mono-substituted amino or di-substituted amino radical.

In some embodiments, the R₂ radical may be hydrogen, or a substituted or unsubstituted organic radical comprising from one to 12 carbon atoms, from one to 6 carbon atoms, or from one to four carbon atoms. Suitable radicals include but are not limited to an alkyl, substituted alkyl, cycloalkyl or substituted cycloalkyl. In some embodiments, the R₂ radical is hydrogen.

In still other embodiments Ar₁ is an aryl or pyridyl radical optionally substituted with hydrogen, alkyl, substituted alkyl, alkenyl, substituted alkenyl, alkynyl, substituted alkynyl, halogen, cyano, nitro, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, alkylsulfonamide, arylsulfonamide, alkylurea, arylurea, alkylcarbamate, arylcarbamate, heteroaryl, haloalkoxy, alkylsulfonyl, alkylsulfinyl, thioalkyl or thiohaloalkyl.

In some other embodiments two substituents together with Ar₁ bonded thereto form a cycloalkyl, substituted cycloalkyl, cycloalkenyl or substituted cycloalkenyl optionally comprising 1 or 2 heteroatoms selected from O, S, SO, SO₂ and N, wherein N is further substituted with hydrogen, or an organic radical having between 1 and 12, or 1 and 6, or 1 and 4 carbon atoms. N- radicals comprising an alkyl or substituted alkyl, haloalkyl, acyl, alkylsufonyl, aryl, substituted aryl, heteroaryl, or substituted heteraryl radical may be beneficial in some embodiments.

In many embodiments, the Ar₁ aryl or heteroaryl ring and/or the additional cyclic ring radical bonded thereto may have 1, 2, 3, 4, 5, 6, or 7 non-hydrogen substituent radicals bonded to one or more ring atoms of the aryl, heteroaryl, or additional cyclic ring radicals. In many embodiments, the additional cyclic ring radical may have between 2 and 5 non-hydrogen substituent radicals bonded thereto.

In many embodiments Ar₁ and its substitutent groups together comprise a total of between 6 to 30 carbon atoms, or 8 to 25 carbon atoms, or 10 to 20 carbon atoms.

In one embodiment the two substituents bonded to Ar₁ are ortho with respect to each other thereby forming a fused ring with Ar₁, one specific example is shown in Formula (II):

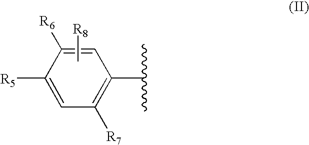

wherein: R₅ and R₆ together with the aromatic ring form a cycloalkyl, substituted cycloalkyl, cycloalkenyl or substituted cycloalkenyl optionally comprising 1 or 2 heteroatoms selected from O, S, SO, SO₂ and N, wherein N is further substituted with hydrogen, alkyl or substituted alkyl; and R₇ and R₈ are independently or together hydrogen, alkyl, substituted alkyl, alkenyl, substituted alkenyl, alkynyl, substituted alkynyl, halogen, cyano, nitro, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, alkylsulfonamide, arylsulfonamide, alkylurea, arylurea, alkylcarbamate, arylcarbamate, heteroaryl, haloalkoxy, alkylsulfonyl, alkylsulfinyl, thioalkyl or thiohaloalkyl.

In one embodiment related to Formula (II), R₅ and R₆ together with the aromatic ring bonded thereto form a 5,6,7,8-tetrahydro-2-naphthyl radical:

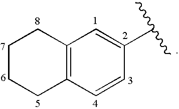

In another embodiment related to Formula (II), R₅ and R₆ together with the aromatic ring bonded thereto form a 5,6,7,8-tetrahydro-2-napthyl radical substituted with alkyl, substituted alkyl, alkenyl, substituted alkenyl, alkynyl, substituted alkynyl, halogen, cyano, nitro, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, alkylsulfonamide, arylsulfonamide, alkylurea, arylurea, alkylcarbamate, arylcarbamate, heteroaryl, haloalkoxy, alkylsulfonyl, alkylsulfinyl, thioalkyl or thiohaloalkyl.

In other embodiments related to Formula (II) R₅ and R₆ together with the aromatic ring bonded thereto form a cycloalkyl or substituted cycloalkyl, such as a polycyclic radical; wherein R₇ is methyl, ethyl, trifluoromethyl, methoxy or dimethylamino; and R₈ is hydrogen. In some embodiments the polycyclic radical is:

1) 3,5,5,8,8-pentamethyl-5,6,7,8-tetrahydro-2-naphthyl,

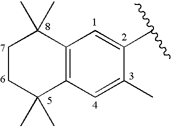

2) 3-ethyl-5,5,8,8-tetrametyl-5,6,7,8-tetrahydro-2-naphthyl,

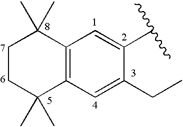

3) 3-trifluoromethyl-5,5,8,8-tetramethyl-5,6,7,8-tetrahydro-2-naphthyl,

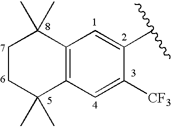

4) 3-methoxy-5,5,8,8-tetramethyl-5,6,7,8-tetrahydro-2-naphthyl, or

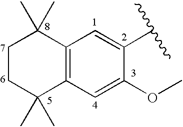

5) 3-dimethylamino-5,5,8,8-tetramethyl-5,6,7,8-tetrahydro-2-naphthyl.

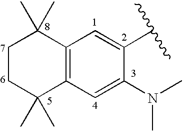

In one embodiment, R₅ and R₆ together with the Ar₁ of Formula (I) form a substituted cycloalkyl with to give the 5,5,8,8-tetramethyl-5,6,7,8-tetrahydro-2-naphthyl radical:

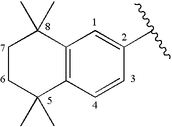

and in still another embodiment, R₅ and R₆ together form a substituted cycloalkyl with the Ar₁ of Formula (I) optionally comprising 1 or 2 nitrogen heteroatoms to give 1-isopropyl-7-methyl-1,2,3,4-tetrahydro-6-quinolinyl radical;

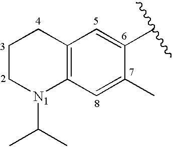

or the 1,4-diisopropyl-6-methyl-1,2,3,4-tetrahydro-7-quinoxalinyl radical:

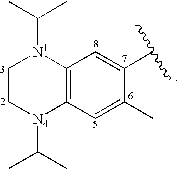

In other embodiments related to Formula (II), at least one of R₅, R₆ and R₈ is an bulky organic substituent, such as an alkyl, a substituted alkyl, a cycloalkyl, a substituted cycloalkyl, a heterocyclic, a substituted heterocyclic, a heteroaryl, a substituted a heteroaryl, an aryl, or a substituted aryl radical. In some preferred embodiments, at least one the R₅, R₆ and R₈ substituents are sterically bulky alkyl or substituted alkyl radicals have the formula

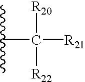

wherein R₂₀, R₂₁, and R₂₂ can be independently selected from hydrogen, alkyl, substituted alkyl, cycloalkyl, substituted cycloalkyl, heterocyclic or substituted heterocyclic ring. Preferably, at least two of the R₂₀, R₂₁, and R₂₂ substituents have carbon atoms bound to the central carbon atom, and no more than one of R₂₀, R₂₁, and R₂₂ are hydrogen, so as to form at least a secondary R₁₂ group. For example, R₂₀ and R₂₁ may both comprise alkyl groups, while R₂₂ is hydrogen. Alternatively, R₂₀ and R₂, may, together with the illustrated carbon atom, form a cycloalkyl, substituted cycloalkyl , heterocyclic or substituted heterocyclic ring, while R₂₂ is an independent substitutent as defined above. In another embodiment, R₂₀ and R₂₁ together with the carbon atom may form an aryl, a substituted aryl, a heteroaryl, or a substituted heteroaryl ring, and R₂₂ would be absent.

Even more preferably, none of R₂₀, R₂₁, and R₂₂ are hydrogen, and R₁₂ therefore comprises a tertiary carbon atom and/or a tertiary group. In many embodiments, the R₅, R₆ and R₈ groups comprises at least 4 carbon atoms, or may comprise between 4 and 15 carbon atoms, or between 4 and 12 carbon atoms.

The bulky substituent radical may be a substituted radical of the Formula:

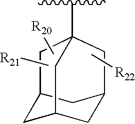

wherein:

R₂₀, R₂₁ and R₂₂ are at any position on the ring radical and are independently hydrogen, halogen, alkyl, hydroxy, carboxyl, alkylcarboxamide or dialkylcarboxamide. In one embodiment R₂₀, R₂₁ and R₂₂ are hydrogen, such that the substituted cycloalkyl is an adamantyl radical of the Formula:

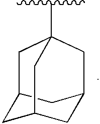

Some embodiments of the invention relate to compounds wherein the bulky substituent radical is a substituted heterocyclic radical of the formula:

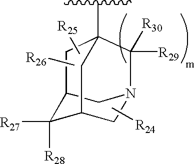

wherein:

m is 0 or 1;

R₂₄, R₂₅ and R₂₆ can be attached to any carbon on the substituted heterocyclic radical except for the carbons bearing R₂₇ and R₂₈ or R₂₉ and R₃₀ and are independently hydrogen, halogen, alkyl, hydroxy, carboxyl, alkylcarboxamide or dialkylcarboxamide;

R₂₇ and R₂₈ are independently hydrogen, halogen, or hydroxy; or R₂₇ and R₂₈ together form a carbonyl radical;

R₂₉ and R₃₀ are independently hydrogen; or R₂₉ and R₃₀ together form a carbonyl radical.

In certain preferred embodiments, the bulky substituent group is a phenyl, a 2-pyridyl, a 3-pyridyl, a 4-pyridyl, a 1-alkylcyclohexyl, or an adamantyl residue.

Additional disclosures regarding non-oxime compounds having bulky substitutents for the Ar₁ groups described in the preceeding several paragraphs are described in co-pending U.S. Utility application Ser. No. 10/094,142, filed Mar. 7, 2002, which is hereby incorporated herein by this referene, in its entirety, for its additional disclosures relating to the structures of similar bulky substituents for Ar₁, and for methods of making precursors and compounds containing such substructure fragments.

In the compounds of the invention, the Ar₂ aryl or heteroaryl aromatic group bearing the oxime sustituent radical may be unsubstituted, or substituted with 0, 1, 2, or 3 additional non-hydrogen substituent groups. Examples of suitable substituent radicals for Ar₂ include one or more of alkyl, substituted alkyl, alkenyl, substituted alkenyl, alkynyl, substituted alkynyl, halogen, cyano, nitro, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, alkylsulfonamide, arylsulfonamide, alkylurea, arylurea, alkylcarbamate, arylcarbamate, heteroaryl, haloalkoxy, alkylsulfonyl, alkylsulfinyl, thioalkyl or thiohaloalkyl radicals. The organic substituent radicals may each independently comprise between 1 and 18 carbon atoms, 1 and 12 carbon atoms, 1 and 6 carbon atoms, or between 1 and 4 carbon atoms.

In some embodiments, Ar₁ is a residue of Formula (200):

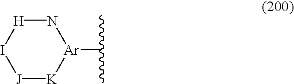

wherein:

a) the B, H, I, J and K residues are independently selected from —C(O)—, —C(S)—, —O—, —S—, —N(R₁₀₁)—,—N(R₁₀₂)—, —C(R₁₀₃)(R₁₀₄)—, —C(R₁₀₅)(R₁₀₆)—, or —C(R₁₀₇)(R₁₀₈)— residues, and from zero to two of the B, H, I, J or K residues may be absent; wherein:

i) R₁₀₁, R₁₀₂, R₁₀₃, R₁₀₄, R₁₀₅, R₁₀₆, R₁₀₇ and R₁₀₈ are independently selected from hydrogen, hydroxyl, a halogen, amino, or an organic residue comprising 1 to 12 carbon atoms; or two of the R₁₀₁, R₁₀₂, R₁₀₃, R₁₀₄, R₁₀₅, R₁₀₆, R₁₀₇ and R₁₀₈ residues may be connected together to form an exocyclic substituent residue comprising 1 to 6 ring carbon atoms and from 0 to 3 optional ring heteroatoms selected from O, S, or N; and

ii) B, H, I, J and K together with the Ar₅ form a ring containing at least one amide residue having the formula

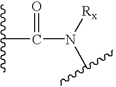

wherein Rₓ is a R₁₀₁ or R₁₀₂ residue.

In some embodiments B, H, I, J and K together with Ar₁ form a ring containing at least one amide residue having the Formulae (205a-k) and Ar₁ is an aryl or substituted aryl:

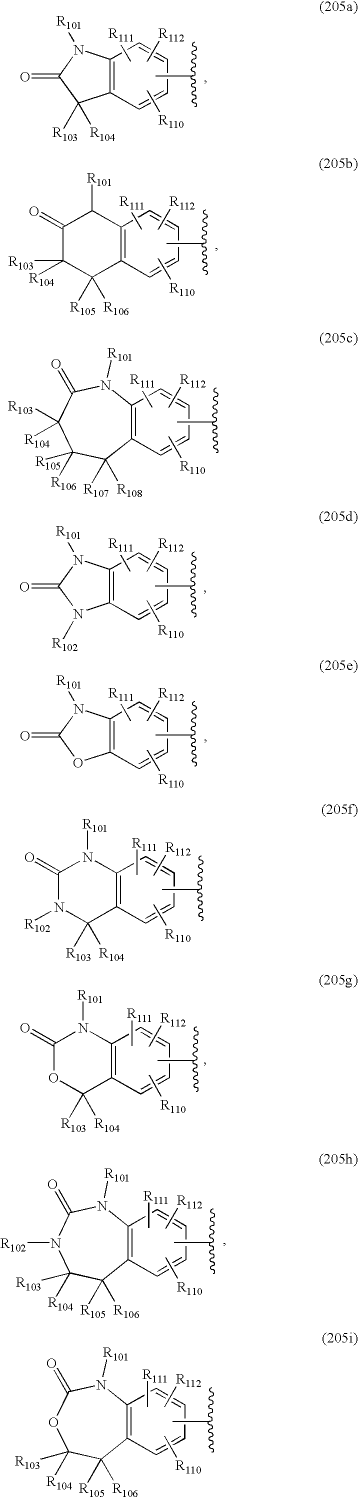

wherein R₁₀₁, R₁₀₂, R₁₀₃, R₁₀₄, R₁₀₅, R₁₀₆, R₁₀₇, R₁₀₈, R₁₁₀, R₁₁₁, or R₁₁₂ may be independently selected from inorganic substitutents, which include but are not limited to inorganic substitutents such as hydrogen, halogen, cyano, nitro, hydroxyl, or amino. Alternatively, R₁₀₁, R₁₀₂, R₁₀₃, R₁₀₄, R₁₀₅, R₁₀₆, R₁₀₇, R₁₀₈, R₁₁₀, R₁₁₁ or R₁₁₂ may comprise an organic residue. Examples of suitable organic residues include but are not limited to an alkyl, substituted alkyl, haloalkyl, alkenyl, substituted alkenyl, alkynyl, substituted alkynyl, acyloxy, amino, mono-substituted amino, di-substituted amino, alkylsulfonamide, arylsulfonamide, alkylurea, arylurea, alkylcarbamate, arylcarbamate, aryl, heteroaryl, alkoxy, substituted alkoxy, haloalkoxy, thioalkyl, thiohaloalkyl, carboxy, carboalkoxy, alkylcarboxamide, substituted alkylcarboxamide, dialkylcarboxamide or substituted dialkylcarboxamide residue. In some embodiments, preferred R₁₀₁, R₁₀₂, R₁₀₃, R₁₀₄, R₁₀₅, R₁₀₆, R₁₀₇, R₁₀₈, R₁₁₀, R₁₁₁ or R₁₁₂ groups are an alkyl, substituted alkyl, haloalkyl, alkoxy, substituted alkoxy, or haloalkoxy residues, particularly those comprising from 1 to 6 carbons, or 1 to four carbons.

Additional disclosures regarding non-oxime compounds having aromatic substructure fragments analogous to the Ar₁ groups described in the preceeding several paragraphs are described in co-pending U.S. Provisional Application Serial No. 60/362,732, filed Mar. 8, 2002, which is hereby incorporated herein by this referene, in its entirety, for its additional disclosures relating to the structures of similar aromatic substructure fragments for Ar₁, and for methods of making precursors and compounds containing such substructure fragments.

In some other embodiments, Ar₁ is a residue of the formula

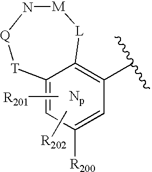

wherein:

a) R₂₀₀, R₂₀₁ and R₂₀₂ are independently selected from hydrogen, hydroxyl, a halogen, amino, or an organic residue comprising 1 to 12 carbon atoms;

b) Nₚ are the number of heteroaryl ring nitrogens selected from 0, 1 or 2;

c) L, M, N, Q and T residues are independently selected from —C(O)—, —C(S)—, —O—, —S—, —N(R₂₀₃)—,—N(R₂₀₄)—, —C(R₂₀₅)(R₂₀₆)—, —C(R₂₀₇)(R₂₀₈)—, or —C(R₂₀₉)(R₂₁₀)— residues, and from zero to two of the L, M, N, Q or T residues may be absent;

wherein:

i) R₂₀₀, R₂₀₁, R₂₀₂, R₂₀₃, R₂₀₄, R₂₀₅, R₂₀₆, R₂₀₇, R₂₀₈, R₂₀₉, and R₂₁₀ are independently selected from hydrogen, hydroxyl, a halogen, amino, or an organic residue comprising 1 to 12 carbon atoms; or two of the R₂₀₃, R₂₀₄, R₂₀₅, R₂₀₆, R₂₀₇, R₂₀₈, R₂₀₉ and R₂₁₀ residues may be connected together to form an exocyclic substituent residue comprising 1 to 6 ring carbon atoms and from 0 to 3 optional ring heteroatoms selected from O, S, or N.

For example, L, M, N, Q and T together with a substituted or unsubstituted aryl may form a ring having the Formulaes (305a-k):

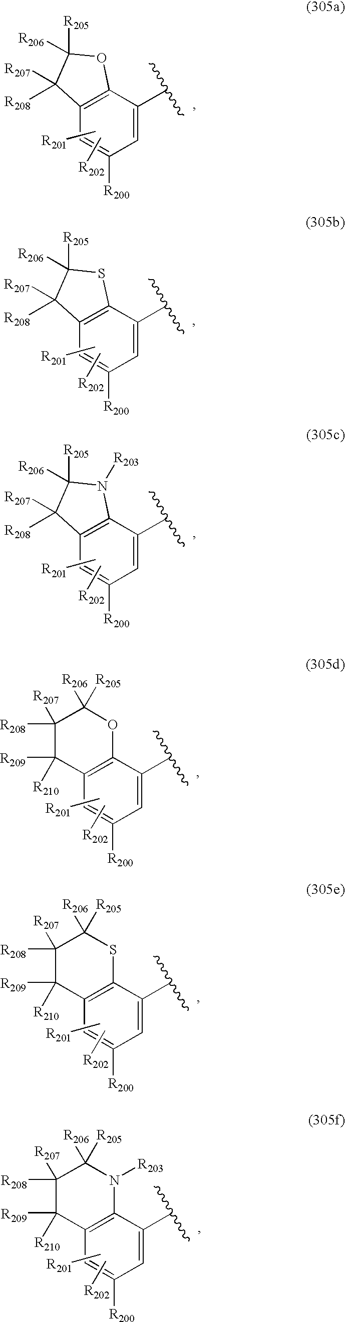

wherein

R₂₀₀, R₂₀₁, R₂₀₂, R₂₀₃, R₂₀₄, R₂₀₅, R₂₀₆, R₂₀₇, R₂₀₈, R₂₀₉, and R₂₁₀ are independently or together hydrogen, alkyl, substituted alkyl, haloalkyl, alkenyl, substituted alkenyl, alkynyl, substituted alkynyl, halogen, cyano, nitro, hydroxyl, acyloxy, amino, mono-substituted amino, di-substituted amino, alkylsulfonamide, substituted alkylsulfonamide, arylsulfonamide, heteroarylsulfonamide, alkylurea, alkylthiourea, arylurea, acyl, substituted acyl, alkylcarbamate, arylcarbamate, alkylthiocarbamate, substituted alkylthiocarbamate, arylthiocarbamate, heteroaryl, substituted heteroaryl, alkoxy, substituted alkoxy, haloalkoxy, thioalkyl, alkylsulfoxide, alkylsulfonyl, thiohaloalkyl, carboxy, carboalkoxy, alkylcarboxamide, substituted alkylcarboxamide, dialkylcarboxamide or substituted dialkylcarboxamide sustituent radicals.

Additional disclosures regarding non-oxime compounds having aromatic substructure fragments analogous to the Ar₁ groups described in the preceeding several paragraphs are described in co-pending U.S. Provisional Application Serial No. 60/362,702, filed Mar. 8, 2002, which is hereby incorporated herein by this referene, in its entirety, for its additional disclosures relating to the structures of similar aromatic substructure fragments for Ar₁, and for methods of making precursors and compounds containing such substructure fragments.

In many embodiments Ar₂ ring and its substitutent groups together comprise a total of between 4 to 20 carbon atoms, or 4 to 15 carbon atoms, or 5 to 10 carbon atoms.

In some embodiments of the invention Ar₂ is of the following formulas:

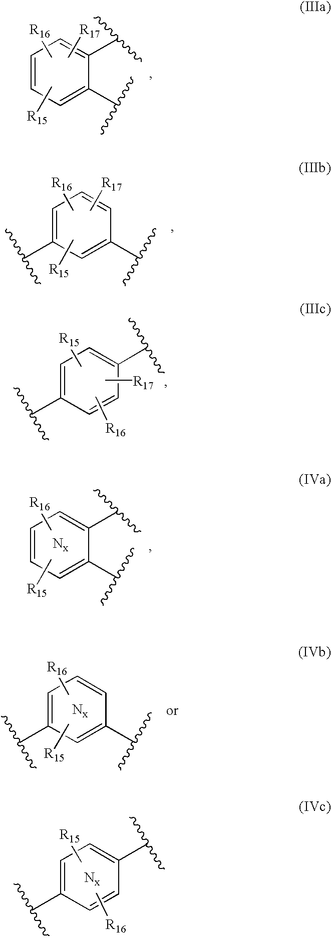

wherein wherein Nₓ is 1 or 2 and the nitrogen atoms are unsubstituted ring atoms, R₁₅, R₁₆ and R₁₇ are independently or together hydrogen, alkyl, substituted alkyl, alkenyl, substituted alkenyl, alkynyl, substituted alkynyl, halogen, cyano, nitro, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, alkylsulfonamide, arylsulfonamide, alkylurea, arylurea, alkylcarbamate, arylcarbamate, heteroaryl, haloalkoxy, alkylsulfonyl, alkylsulfinyl, thioalkyl or thiohaloalkyl.

Formulaes (IVa), (IVb) and (IVc) represent different nitrogen containing heteroaryl radicals for Ar₂, wherein one or two ring nitrogens are present and can be at any position not already substituted with another group. By way of an example, when one ring nitrogen is present in Formula (IVb) the following structures are within the scope of the invention:

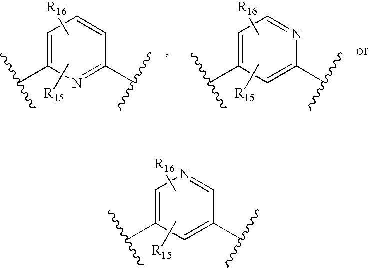

Similarly, when two ring nitrogens are present in Formula (IVb) the following structures are within the scope of the invention:

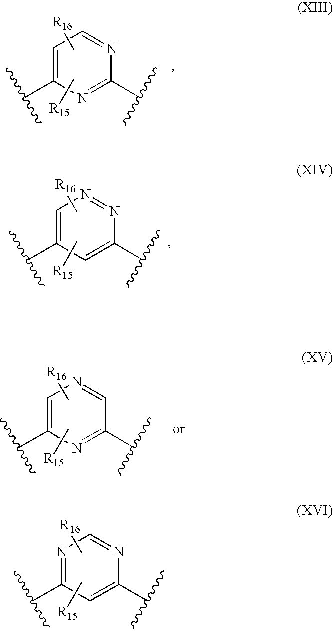

and R₁₅ and R₁₆ have the same definition as above.

It is understood that the Formulaes disclosed herein are general structures and where applicable can represent more than one bonding orientation with respect to other radicals in Formula (I) and other embodiments disclosed herein, such as, for example, Formula (X), can represent either Formula (XI) or Formula (XII):

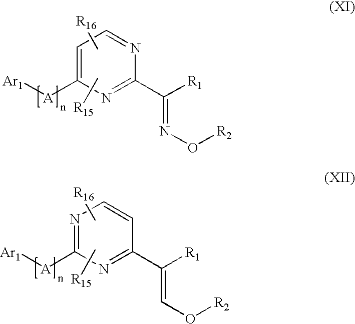

wherein Ar₁ A, n, R₁ and R₂ have the same meaning as defined above.

In other embodiments n is 1 and A is an alkylene optionally comprising 1 or 2 heteroatoms selected from 0 or N, wherein N is further substituted with hydrogen or alkyl.

In one embodiment R₁ is hydrogen, alkyl or substituted alkyl.

In another embodiment R₂ is hydrogen, alkyl or substituted alkyl.

In still another embodiment R₁ or R₂ are hydrogen.

This invention also relates to a pharmaceutical formulation comprising one or more compounds disclosed herein in an admixture with a pharmaceutically acceptable excipient.

Certain compounds disclosed herein (especially the oxime and oxime derivatives of the invention) may exist in either the pure syn or pure anti configuration or a mixture of syn and anti configurations. Both configurations and/or mixtures thereof are within the scope of the invention.

The compounds disclosed herein may also include salts of the compounds, such as salts with cations. Cations with which the compounds of the invention may form pharmaceutically acceptable salts include alkali metals, such as sodium or potassium; alkaline earth metals, such as calcium; and trivalent metals, such as aluminum. The only constraint with respect to the selection of the cation is that it should not unacceptably increase the toxicity. Also, compounds disclosed herein may include salts formed by reaction of a nitrogen contained within the compound, such as an amine, aniline, substituted aniline, pyridyl and the like, with an acid, such as HCl, carboxylic acid and the like.

The present invention provides, but is not limited to, the specific compounds set forth in the Examples as well as those set forth below, and a pharmaceutically acceptable salt thereof:

3-(3,5,5,8,8-Pentamethyl-5,6,7,8-tetrahydro-2-naphthyl)-4-methoxybenzaldehyde oxime,

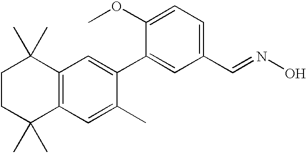

3-(3,5,5,8,8-Pentamethyl-5,6,7,8-tetrahydro-2-naphthyl)-4-trifluoromethoxybenzaldehyde oxime,

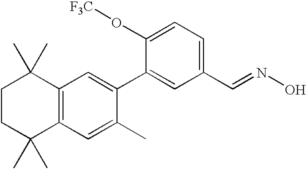

3-(3,5,5,8,8-Pentamethyl-5,6,7,8-tetrahydro-2-naphthyl)-4-dimethylaminobenzaldehyde oxime,

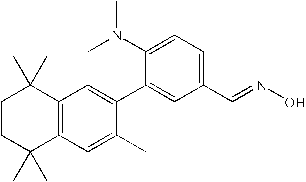

3-(3,5,5,8,8-Pentamethyl-5,6,7,8-tetrahydro-2-naphthyl)-2-fluoro-4-methoxybenzaldehyde oxime,

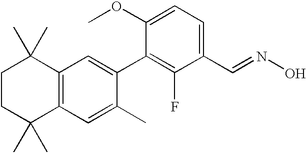

5-(3,5,5,8,8-Pentamethyl-5,6,7,8-tetrahydro-2-naphthyl)-6-methoxy-3-pyridinecarboxaldehyde oxime,

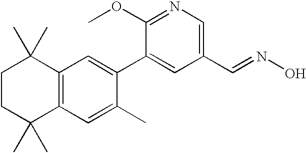

6-(3,5,5,8,8-Pentamethyl-5,6,7,8-tetrahydro-2-naphthyl)-5-methoxy-2-pyridinecarboxaldehyde oxime,

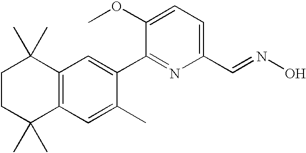

3-(3,5,5,8,8-Pentamethyl-5,6,7,8-tetrahydro-2-naphthyl)-4-methoxy-6-hydroxybenzaldehyde oxime,

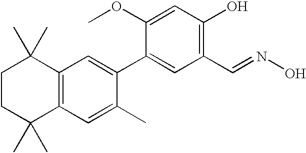

3-(3,5,5,8,8-Pentamethyl-5,6,7,8-tetrahydro-2-naphthyl)-4,6-dimethoxybenzaldehyde oxime,

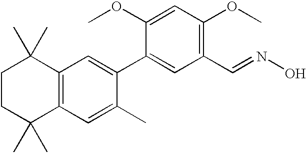

3-(3,5,5,8,8-Pentamethyl-5,6,7,8-tetrahydro-2-naphthyl)-4,6-dihydroxybenzaldehyde oxime,

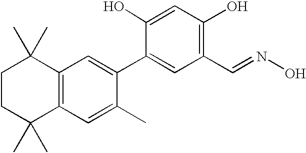

3-(1,4-Diisopropyl-6-methyl-1,2,3,4-tetrahydro-7-quinoxalinyl)-4-methoxybenzaldehyde oxime,

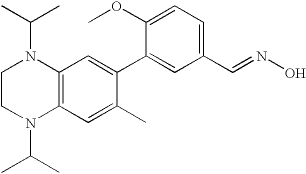

3-(5,5,8,8-Tetramethyl-5,6,7,8-tetrahydro-2-naphthyl)-2,4-dimethoxybenzaldehyde oxime,

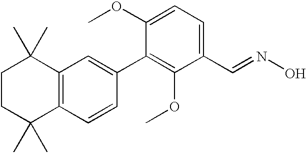

3-(5,5,8,8-Tetramethyl-5,6,7,8-tetrahydro-2-naphthyl)-4-methoxybenzaldehyde oxime,

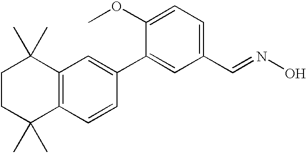

3-(1-Isopropyl-7-methyl-1,2,3,4-tetrahydro-6-quinolinyl)-4-methoxybenzaldehyde oxime,

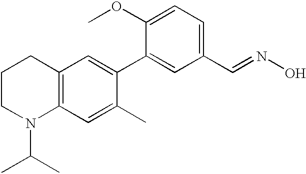

3-(3,5,5,8,8-Pentamethyl-5,6,7,8-tetrahydro-2-naphthyl)-4,5-dimethoxybenzaldehyde oxime,

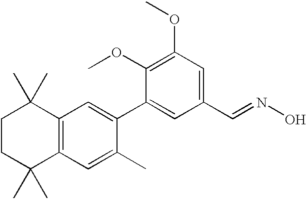

3-(3,5,5,8,8-Pentamethyl-5,6,7,8-tetrahydro-2-naphthyl)-4-hydroxybenzaldehyde oxime,

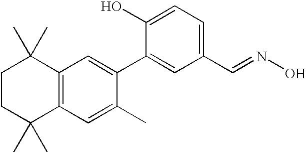

3-(3,5,5,8,8-Pentamethyl-5,6,7,8-tetrahydro-2-naphthyl)-4-methoxy-5-fluorobenzaldehyde oxime,

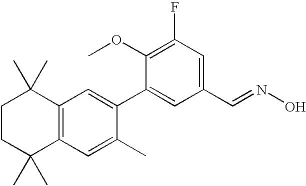

3-(3,5,5,8,8-Pentamethyl-5,6,7,8-tetrahydro-2-naphthyl)-4-methoxy-2,5-difluorobenzaldehyde oxime,

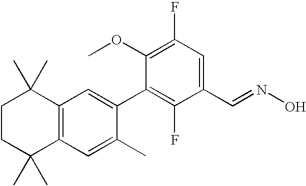

3-(3,5,5,8,8-Pentamethyl-5,6,7,8-tetrahydro-2-naphthyl)-4-chlorobenzaldehyde oxime,

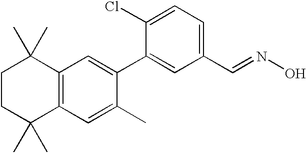

3-(3,5,5,8,8-Pentamethyl-5,6,7,8-tetrahydro-2-naphthyl)-4-methylbenzaldehyde oxime,

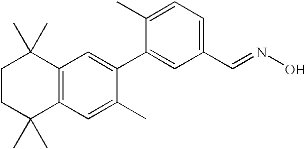

3-(3,5,5,8,8-Pentamethyl-5,6,7,8-tetrahydro-2-naphthyl)-4-ethylbenzaldehyde oxime,

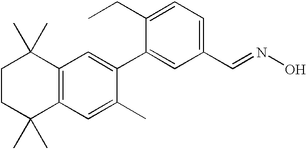

3-(3,5,5,8,8-Pentamethyl-5,6,7,8-tetrahydro-2-naphthyl)-4-trifluoromethylbenzaldehyde oxime,

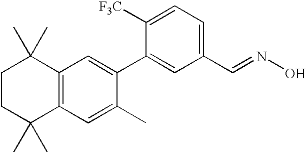

3-(3,5,5,8,8-Pentamethyl-5,6,7,8-tetrahydro-2-naphthyl)-4-ethoxybenzaldehyde oxime,

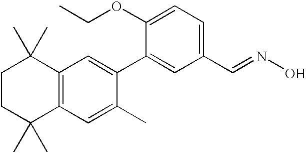

3-(3,5,5,8,8-Pentamethyl-5,6,7,8-tetrahydro-2-naphthyl)-4-ethoxy-2-fluorobenzaldehyde oxime,

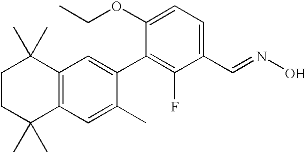

3-(3,5,5,8,8-Pentamethyl-5,6,7,8-tetrahydro-2-naphthyl)-4-isopropoxybenzaldehyde oxime,

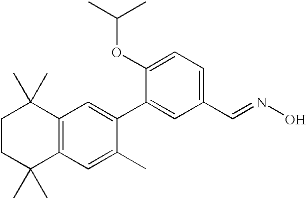

3-(3,5,5,8,8-Pentamethyl-5,6,7,8-tetrahydro-2-naphthyl)-4-methylamino-5-bromobenzaldehyde oxime,

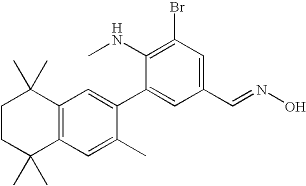

3-(3,5,5,8,8-Pentamethyl-5,6,7,8-tetrahydro-2-naphthyl)-4-aminobenzaldehyde oxime,

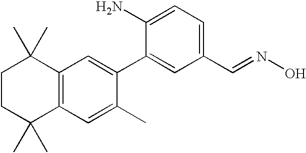

3-(3,5,5,8,8-Pentamethyl-5,6,7,8-tetrahydro-2-naphthyl)-4-acetamidobenzaldehyde oxime,

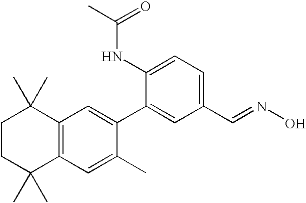

3-(3,5,5,8,8-Pentamethyl-5,6,7,8-tetrahydro-2-naphthyl)-4-ethoxy-2,5-difluorobenzaldehyde oxime,

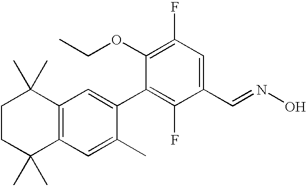

3-(3-Methoxy-5,5,8,8-tetramethyl-5,6,7,8-tetrahydro-2-naphthyl)-4-trifluoromethoxybenzaldehyde oxime,

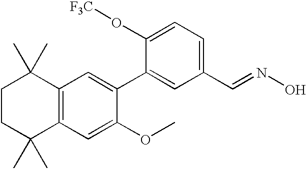

3-(3-Dimethylamino-5,5,8,8-tetramethyl-5,6,7,8-tetrahydro-2-naphthyl)-4-trifluoromethoxybenzaldehyde oxime,

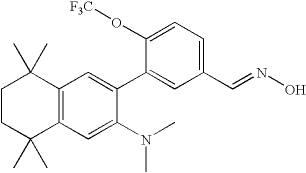

3-(3-Trifluoromethyl-5,5,8,8-tetramethyl-5,6,7,8-tetrahydro-2-naphthyl)-4-methoxybenzaldehyde oxime,

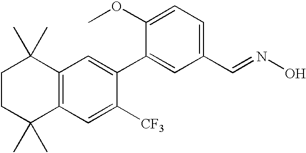

3-(3-Trifluoromethyl-5,5,8,8-tetramethyl-5,6,7,8-tetrahydro-2-naphthyl)-4-methylbenzaldehyde oxime,

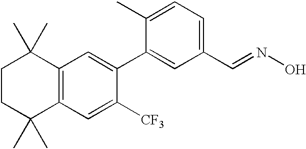

3-(3-Trifluoromethyl-5,5,8,8-tetramethyl-5,6,7,8-tetrahydro-2-naphthyl)-4-ethylbenzaldehyde oxime,

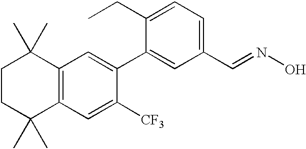

3-(3-Trifluoromethyl-5,5,8,8-tetramethyl-5,6,7,8-tetrahydro-2-naphthyl)-4-trifluoromethoxybenzaldehyde oxime,

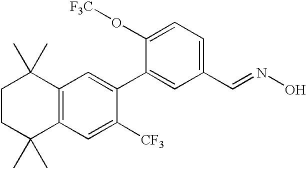

3-(3-Dimethylamino-5,5,8,8-tetramethyl-5,6,7,8-tetrahydro-2-naphthyl)-4-dimethylaminobenzaldehyde oxime,

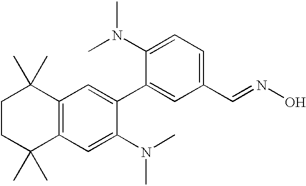

3-(3-Trifluoromethyl-5,5,8,8-tetramethyl-5,6,7,8-tetrahydro-2-naphthyl)-4-chlorobenzaldehyde oxime,

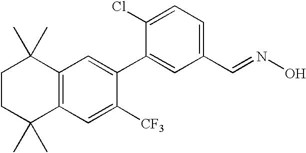

3-(3,5,5,8,8-Pentamethyl-5,6,7,8-tetrahydro-2-naphthyl)-4-acetoxybenzaldehyde oxime,

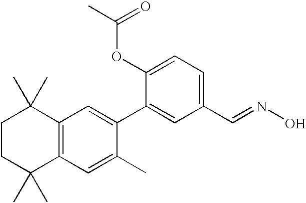

3-(3,5,5,8,8-Pentamethyl-5,6,7,8-tetrahydro-2-naphthyl)-2-fluoro-4-hydroxybenzaldehyde oxime,

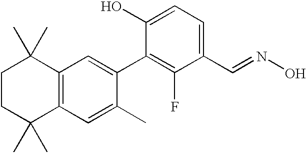

3-(3,5,5,8,8-Pentamethyl-5,6,7,8-tetrahydro-2-naphthyl)-4-(1-propen-3-yl)-benzaldehyde oxime,

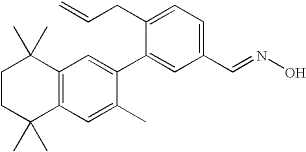

3-(3,5,5,8,8-Pentamethyl-5,6,7,8-tetrahydro-2-naphthyl)-4-hydroxy-5-fluorobenzaldehyde oxime,

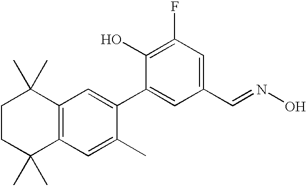

3-(3,5,5,8,8-Pentamethyl-5,6,7,8-tetrahydro-2-naphthyl)-4-hydroxy-2,5-difluorobenzaldehyde oxime,

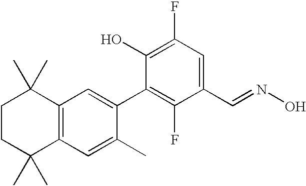

3-(3,5,5,8,8-Pentamethyl-5,6,7,8-tetrahydro-2-naphthyl)-4-methoxy-2,6-difluorobenzaldehyde oxime,

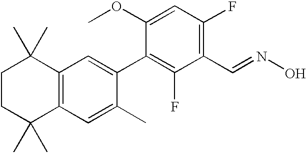

3-(3,5,5,8,8-Pentamethyl-5,6,7,8-tetrahydro-2-naphthyl)-4-methoxy-5,6-difluorobenzaldehyde oxime,

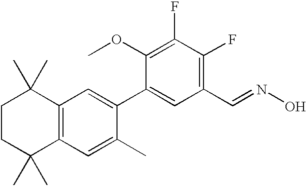

3-(3-Trifluoromethyl-5,5,8,8-tetramethyl-5,6,7,8-tetrahydro-2-naphthyl)-4-dimethylaminobenzaldehyde oxime,

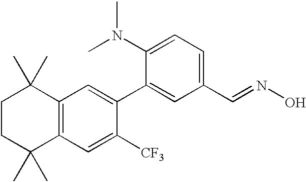

3-(3,5-Di-t-butyl-4-hydroxyphenyl)-4-methoxybenzaldehyde oxime,

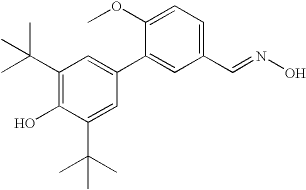

3-(3-Ethyl-5,5,8,8-tetramethyl-5,6,7,8-tetrahydro-2-naphthyl)-4-trifluoromethoxybenzaldehyde oxime,

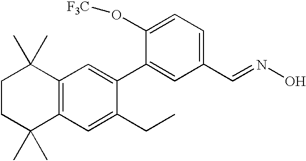

2-(3,5,5,8,8-Pentamethyl-5,6,7,8-tetrahydro-2-naphthyl)-3-methoxybenzaldehyde oxime,

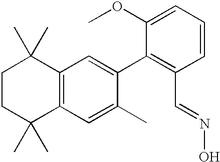

4-[3-(3,5,5,8,8-Pentamethyl-5,6,7,8-tetrahydro-2-naphthyl)-4-trifluoromethoxy-benzyloxy]benzaldehyde oxime

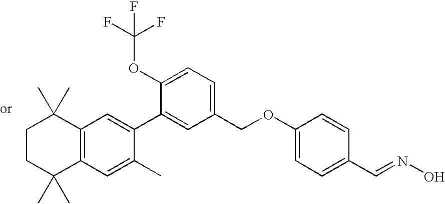

4-[2-(Methyl-pyridin-2-yl-amino)-ethoxy]benzaldehyde oxime

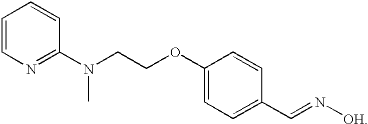

Making the Compositions

Various synthetic methods may be employed by those of skill in the art in the production of the compounds and intermediates disclosed herein. For example, the many known methods of organic chemistry may be employed to provide precursor carbonyl compounds as shown below,

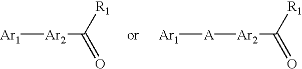

wherein Ar₁, Ar₂, and/or R₁ may be as described in any of the embodiments herein. These precursor carbonyl compounds could be prepared, for example, by acylation of the corresponding aromatic compounds, or other alternative methods. The precursor carbonyl compounds could then be treated with hydroxylamine, or a derivative thereof, to provide the desired oxime or oxime derivative compounds of the invention

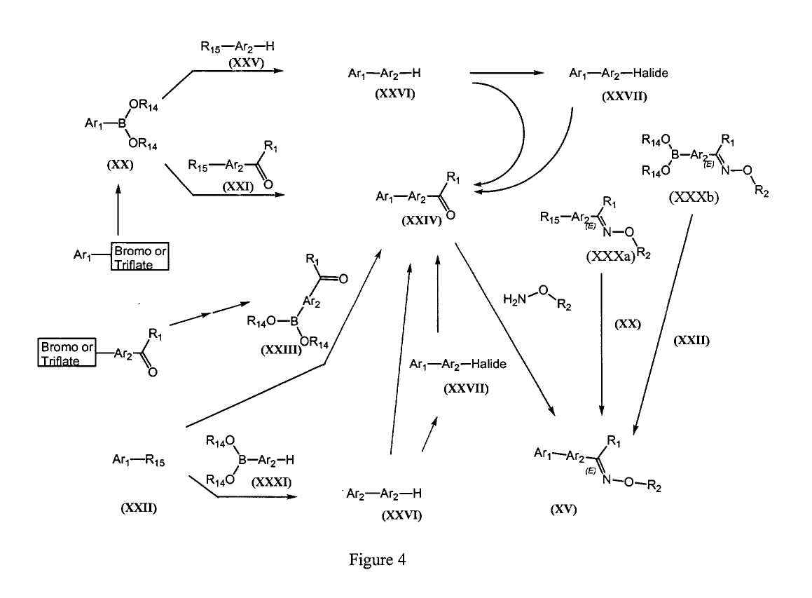

A representative set of synthetic pathways are shown in FIG. 4 when n=0 (i.e. no bridging A group is present. One method, for example, includes coupling a boronic acid of Formula (XX), R₁₄=H, with a carbonyl-containing aryl bromide of Formula (XXI), R₁₅=Br, to give biaryl (XXIV) that is substituted with a carbonyl group, such as, for example, a formyl group (i.e., R₁=H). Alternatively, boronic acid (XX) may be coupled with aryl bromide (XXV), R₁₅=Br, to give biaryl (XXVI) that is subsequently formylated using techniques known in the art, such as the Vilsmeier or the Vilsmeier-Haack reaction, the Gatterman reaction, the Duff reaction, the Reimer-Tiemann reaction or a like reaction. Coupling reactions such as that described for the formation of Biaryl (XXIV) and (XXVI) may also be conducted using boronic esters, such as where R₁₄ together with the boron form a pinacol borate ester (formation of pinacol esters: Ishiyama, T., et al., J. Org. Chem. 1995, 60, 7508-7510, Ishiyama, T., et al., Tetrahedron Letters 1997, 38, 3447-3450; coupling pinacol esters: Firooznia, F. et al., Tetrahedron Letters 1999, 40, 213-216, Manickam, G. et al., Synthesis 2000, 442-446; all four citations incorporated herein by reference). In addition, R₁₅ may also be I, Cl or triflate (derived from a phenol). Biaryl (XXVI) may also be acylated, for example by the Friedel-Crafts Acylation reaction or the like. In one embodiment, the biaryl (XXVI) is formylated. Alternatively, in a two step manner, biaryl (XXVI) is formylated by first performing a halogenation step to give biaryl (XXVII), such as a bromination, followed by a halogen-metal exchange reaction using an alkyl lithium and reaction with DMF or equivalent known in the art to give biaryl (XXIV) where R₁ is H. The carbonyl group of biaryl (XXIV) may subsequently be condensed with a hydroxylamine derivative to give biaryl (XV). In one embodiment the hydroxylamine derivative is hydroxylamine (i.e., R₂=H).

In an alternative manner, the coupling may take place between aryl (XXII), such as, for example, where R₁₅=Br, and boronic acid (XXIII, R₁₄=H) to give the above mention biaryl (XXIV). Also aryl (XXII) may be coupled with boronic acid (XXXI) to give biaryl (XXVI). Employing the same strategy as described above biaryl (XXVI) may be either formylated or acylated to achieve biaryl (XXIV).

Aryl (XX) can be readily produced by reaction of Ar₁-Halide, such as bromide, with an alkyl lithium to give the Ar₁-lithium that is subsequently allowed to react with a borate ester and hydrolysized to give aryl (XX) wherein R₁₄ is hydrogen. In another method, aryl (XX) can be prepared by reacting Ar₁-Triflate with a pinacoldiboron in the presence of a pallidium catalyst, such as, dppf, to give the corresponding aryl (XX) wherein the two R₁₄ groups together with the boron form a pinacol ester. In another embodiment, aryl (XXIII) can be readily obtained by first protecting the carbonyl group using methods known in the art, such as, for example, an acetal or ketal, and then reacting the halide, such as a bromide, with an alkyl lithium to give the Ar₂-lithium that is subsequently allowed to react with a borate ester and hydrolysized to deprotect the carbonyl group and give aryl (XXIII) wherein R₁₄ is hydrogen. In another method, aryl (XXIII) can be prepared without protection of the carbonyl group by reacting Ar₂-Triflate with a pinacoldiboron in the presence of a palladium catalyst, such as, dppf, to give the corresponding aryl (XXIII) wherein the two R₁₄ groups together with the boron form a pinacol ester.

Some embodiments of the invention relate to compound of Formula (XV):

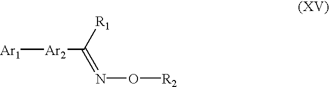

and processes for their preparation, wherein:

Ar₁ is a substituted or unsubstituted aryl radical or a substituted or unsubstituted heteroaryl radical;

Ar₂ is a substituted or unsubstituted aryl radical or a substituted or unsubstituted heteroaryl radical;

A is a substituted or unsubstituted bridging radical comprising a connected chain of atoms comprising from one to nine carbon atoms and optionally comprising one or two heteroatoms selected from O, S and N, wherein N is further substituted with hydrogen, alkyl or substituted alkyl;

R₁ is hydrogen, a substituted or unsubstituted amino radical, or a substituted or unsubstituted organic radical comprising from one to 12 carbon atoms; and

R₂ is hydrogen, or a substituted or unsubstituted organic radical comprising from one to 12 carbon atoms;

or a pharmaceutically acceptable salt thereof;

comprising the steps of:

i) coupling an Ar₁ precursor compound with an Ar₂ precursor compound to give a biaryl carbonyl containing compound; wherein:

(1) the Ar₁ precursor compound has the structure:

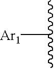

(2) and the Ar₂ precursor compound has a carbonyl group and has the structure:

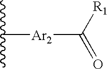

(3) and wherein the biaryl carbonyl containing compound has the structure:

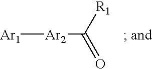

ii) condensing the biaryl carbonyl containing compound with a hydroxylamine derivative having the structure:

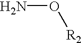

to give a compound of Formula (XV), or a pharmaceutically acceptable salt thereof.

In another embodiment of the invention relates to a process wherein the Ar₁ radical is of the Formula:

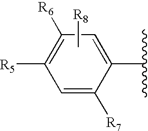

In still another embodiment of the invention relates to a process wherein the hydroxylamine derivative is of the formula:

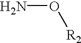

wherein R₂ is hydrogen.

The various organic group transformations utilized herein may be performed by a number of procedures other than those described above. References for other synthetic procedures that may be utilized for the synthetic steps leading to the compounds disclosed herein may be found in, for example, March, J., Advanced Organic Chemistry, 5^{th} Edition, Weiley-Interscience (2001); or Larock, R. C., Comprehensive Organic Transformations, A Guide to Functional Group Preparations, 2^{nd} Edition, VCH Publishers, Inc. (1999), both incorporated herein by reference, for their disclosures of the known reaction and methods of organic chemistry that might be employed to make the compounds of the invention.

One embodiment of the invention relates to the processes for making compounds of Formula I, wherein n is 0, which comprises coupling two aromatic rings to give a biaryl wherein one of the aryl rings contains a carbonyl moiety, in another embodiment the carbonyl moiety is an aldehyde. The resulting biaryl product may be subsequently condensed with an hydroxylamine derivative, such as hydroxylamine to give a compound of Formula (I).

In another embodiment of the invention, wherein n is 0, relates to the process of making compounds of Formula (I) which comprises coupling two aromatic rings to give a biaryl wherein one of the aryl rings, such as Ar₂, contains an oxime moiety to give a compound of Formula (I). In this embodiment the condensation with the hydroxylamine derivative takes place prior to the coupling of two aromatic rings.

Coupling of two aryl rings may be conducted using an aryl boronic acid or esters with an aryl halide (such as, iodo, bromo, or chloro), triflate or diazonium tetrafluoroborate; as described respectively in Suzuki, Pure & Applied Chem., 66:213-222 (1994), Miyaura and Suzuki, Chem. Rev. 95:2457-2483 (1995), Watanabe, Miyaura and Suzuki, Synlett. 207-210 (1992), Littke and Fu, Angew. Chem. Int. Ed., 37:3387-3388 (1998), Indolese, Tetrahedron Letters, 38:3513-3516 (1997), Firooznia, et. al., Tetrahedron Letters 40:213-216 (1999), and Darses, et. al., Bull. Soc. Chim. Fr. 133:1095-1102 (1996); all incorporated herein by reference. According to this coupling reaction, precursors such as (XX) and (XXI) may be used or in another embodiment of the invention (XXa) and (XXI) may be used:

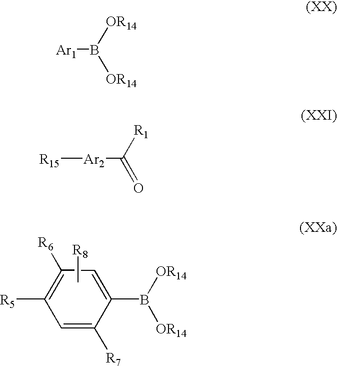

wherein R₁₄ is either alkyl or hydrogen and R₁₅ is a halide (such as, iodo, bromo, or chloro), triflate or diazonium tetrafluoroborate. Alternately, it is understood that the coupling groups may be reversed, such as, for example, the use of (XXIIa) and (XXIII), or, in another embodiment, (XXIIb) and (XXIII) to achieve the same coupling product:

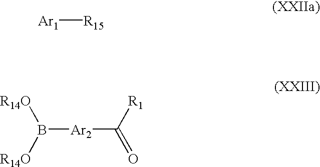

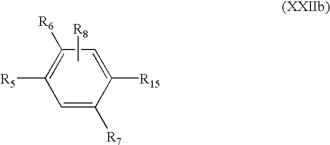

wherein R₁₄ and R₁₅ have the same meaning as described above. The preparation of the above mentioned precursors may be prepared by methods readily available to those skilled in the art. For example, the boronic ester may be prepared from an aryl halide by conversion into the corresponding aryl lithium, followed by treatment with a trialkyl borate. Preferably, the boronic ester is hydrolyzed to the boronic acid.

The coupling of the two aromatic rings may be accomplished in a similar manner using compound (XXXa) and compound (XXa) or (XXb) to give a compound of Formula (I) wherein n=0. Alternatively, compound (XXXb) and compound (XXIIa) or (XXIIb) may be coupled to give a compound of Formula (I) wherein n=0. In this process, the condensation takes place prior to the coupling of the two aromatic rings.

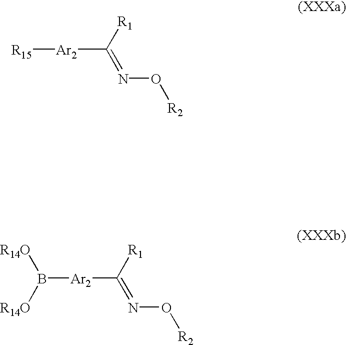

The coupling reaction may also be conducted between an arylzinc halide and an aryl halide or triflate. Alternately, the coupling reaction may also be executed using an aryl trialkyltin derivative and an aryl halide or triflate. These coupling methods are reviewed by Stanforth, Tetrahedron 54:263-303 (1998) and incorporated herein by reference. In general, the utilization of a specific coupling procedure is selected with respect to available precursors, chemoselectivity, regioselectivity and steric considerations.

Condensation of the biaryl carbonyl containing derivatives (e.g., FIG. 4, compound (XXIV)) with a suitable hydroxylamine derivative, such as, hydroxylamine, may be accomplished by the use of methods known in the art. For example, the biaryl carbonyl product from the coupling reaction may be condensed with a hydroxylamine derivative to give a compound of Formula (I). This type of condensation may be solvent and pH dependent and it is understood that routine experimentation may be necessary to identify the optimal solvent with a particular base, for example, pyridine, triethylamine and the like, and a solvent such as an alcohol, for example, ethanol and the like; or mixtures thereof.

Using the Compositions

The compounds disclosed herein can function, for example, as antidiabetic molecules, modulators of lipid metabolism, and/or carbohydrate metabolism. This activity can be demonstrated in animal models of dyslipidemia and type 2 diabetes, such as in the Zuker fatty rat or the KKA^{y} mouse. In these models a compound is considered active if it is able to exhibit the ability to reduce glucose or increase glucose tolerance compared to controls. Compounds disclosed herein can be useful, for example, to modulate metabolism (such as, for example, lipid metabolism and carbohydrate metabolism) and can be used to treat type 2 diabetes. Modulation of lipid metabolism could also include a decrease of lipid content intracellularly or extracellularly. Modulation of lipid metabolism could also include the increase of one type of lipid containing particle such as high density lipoprotein (HDL) and or simultaneous decrease in low density lipoprotein (LDL). In one suitable animal model to measure such activity in vivo, young Sprague Dawley rats fed a high cholesterol diet. Modulation of metabolism may occur directly for example, through binding of the compounds disclosed herein with its cognate nuclear receptor, which directly affects an increase or decrease in lipid content by up-regulation or down-regulation of a gene involved in lipid metabolism. Modulation, for example, could be an increase in lipid metabolism, such that lipid metabolism is greater than that of a control. Modulation, also includes, for example, an increase in lipid metabolism, such that the lipid metabolism approaches that of a control. Likewise, modulation of lipid metabolism could be a decrease in lipid metabolism, such that the lipid metabolism is less than or decreasing towards a control. Carbohydrate metabolism can also be up-regulated or down-regulated to either approach the level of carbohydrate metabolism in a control or to deviate from the level of carbohydrate metabolism in a control. Changes in carbohydrate metabolism can directly or indirectly also result in changes of lipid metabolism and, similarly, changes in lipid metabolism can lead to changes in carbohydrate metabolism. An example is type 2 diabetes where an increase in free fatty acids in the patients leads to decreased cellular uptake and metabolism of glucose.

It is understood that a variety of lipid molecules can be modulated. The compounds disclosed herein can modulate a single type of lipid molecule, such as cholesterol, or the compounds disclosed herein can modulate multiple types of lipid molecules. The compounds disclosed herein can also modulate a single or variety of carbohydrate molecules. The compounds disclosed herein can modulate metabolism disorders, such as dylipidemia. Metabolism can be modulated by the compounds disclosed herein by, for example, decreasing the serum cholesterol and/or the serum triglyceride levels, relative to a control having serum cholesterol and/or triglyceride levels indicative of a mammal having dyslipidemia or hypercholesteremia. It is recognized that any decrease in serum cholesterol and/or triglyceride levels can benefit the mammal having dyslipidemia.

These compounds may be characterized by their low molecular weights and physiological stability, and therefore, represent a class that can be implemented to prevent, alleviate, and/or otherwise, treat disorders of lipid and carbohydrate metabolism, such as obesity, dyslipidemia, type 2 diabetes and other diseases related to type 2 diabetes. It is understood that treatment or prevention of type 2 diabetes can involve modulation of lipid or carbohydrate metabolism, such as the modulation of serum glucose or serum triglyceride levels.

An embodiment of the invention relates to the use of the compounds disclosed herein. The compounds disclosed herein can be either used singularly or plurally, and pharmaceutical compositions thereof for the treatment of mammalian diseases, particularly those related to humans. Compounds disclosed herein and compositions thereof can be administered by various methods including, for example, orally, internally, parentally, topically, nasally, vaginally, ophthalinically, sublingually or by inhalation for the treatment of diseases related to lipid metabolism, such as dyslipidemia and hypercholesteremia, carbohydrate metabolism, lipid and carbohydrate metabolism such as polycystic ovary syndrome, syndrome X, type 2 diabetes, including disorders related to type 2 diabetes such as, diabetic retinopathy, neuropathy, macrovascular disease or differentiation of adipocytes. Routes of administration and dosages known in the art may be found in Comprehensive Medicinal Chemistry, Volume 5, Hansch, C. Pergamon Press, 1990; incorporated herein by reference.

Although the compounds described herein may be administered as pure chemicals, it is preferable to present the active ingredient as a pharmaceutical composition. Thus another embodiment of the disclosed compounds is the use of a pharmaceutical composition comprising one or more compounds and/or a pharmaceutically acceptable salt thereof, together with one or more pharmaceutically acceptable carriers thereof and, optionally, other therapeutic and/or prophylactic ingredients. The carrier(s) must be ‘acceptable’ in the sense of being compatible with the other ingredients of the composition and not overly deleterious to the recipient thereof.

Pharmaceutical compositions typically include those suitable for oral, enterable, parental (including intramuscular, subcutaneous and intravenous), topical, nasal, vaginal, ophthalinical, sublingually or by inhalation administration. The compositions may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combination thereof, and then, if necessary, shaping the product into the desired delivery system.

Pharmaceutical compositions suitable for oral administration may be presented as discrete unit dosage forms such as hard or soft gelatin capsules, cachets or tablets each containing a predetermined amount of the active ingredient; as a powder or as granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art., e.g., with enteric coatings.

Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or one or more preservative.

The compounds may also be formulated for parenteral administration (e.g., by injection, for example, bolus injection or continuous infusion) and may be presented in unit dose form in ampules, pre-filled syringes, small bolus infusion containers or in multi-does containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds may be formulated as ointments, creams or lotions, or as the active ingredient of a transdermal patch. Suitable transdermal delivery systems are disclosed, for example, in Fisher et al. (U.S. Pat. (No. 4,788,063, incorporated herein by reference) or Bawa et al. (U.S. Pat. Nos. 4,931,279, 4,668,506 and 4,713,224; all incorporated herein by reference). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredient may also be delivered via iontophoresis, e.g., as disclosed in U.S. Pat. Nos. 4,140,122, 4,383,529, or 4,051,842; incorporated herein by reference.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions may be adapted to provide sustained release of the active ingredient employed, e.g., by combination thereof with certain hydrophilic polymer matrices, e.g., comprising natural gels, synthetic polymer gels or mixtures thereof.

The pharmaceutical compositions according to the invention may also contain other adjuvants such as flavorings, coloring, antimicrobial agents, or preservatives.

It will be further appreciated that the amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, one of skill in the art understands how to extrapolate in vivo data obtained in a model organism, such as mouse, rat and the like, to another mammal, such as a human. These extrapolations are not simply based on the weights of the two organisms, but rather incorporate differences in metabolism, differences in pharmacological delivery, and administrative routes. Based on these types of considerations, a suitable dose will, in alternative embodiments, typically be in the range of from about 0.5 to about 100 mg/kg/day, from about 1 to about 75 mg/kg of body weight per day, from about 3 to about 50 mg per kilogram body weight of the recipient per day, or in the range of 6 to 90 mg/kg/day, or typically in the range of 15 to 60 mg/kg/day.

The compound is conveniently administered in unit dosage form; for example, in alternative embodiments, containing typically 0.5 to 1000 mg, 5 to 750 mg, most conveniently, or 10 to 500 mg of active ingredient per unit dosage form.

One skilled in the art will recognize that dosage and dosage forms outside these typical ranges can be tested and, where appropriate, be used in the methods of this invention.

In separate embodiments, the active ingredient may be administered to achieve peak plasma concentrations of the active compound of from typically about 0.5 to about 75 μM, about 1 to 50 μM, or about 2 to about 30 μM. This may be achieved, for example, by the intravenous injection of a 0.05 to 5% solution of the active ingredient, optionally in saline, or orally administered as a bolus containing about 0.5-500 mg of the active ingredient. Desirable blood levels may be maintained by continuous infusion to provide about 0.01-5.0 mg/kg/hr or by intermittent infusions containing about 0.4-15 mg/kg of the active ingredients.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

The following examples are given to illustrate the invention and are not intended to be inclusive in any manner:

### EXAMPLES

### Example 1

3-(3,5,5,8,8-Pentamethyl-5,6,7,8-tetrahydro-2-naphthyl)-4-methoxybenzaldehyde oxime, also referred to Compound 1 herein:

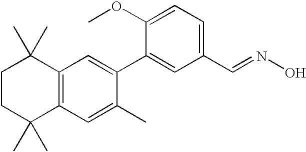

To a solution of 4-methoxy-3-(3,5,5,8,8-pentamethyl-5,6,7,8-tetrahydronaphthylen-2-yl)benzaldehyde (0.300 g, 0.89 mmol, 1 eq) in ethanol (10 mL) was added hydroxylamine hydrochloride (0.065 g, 0.89 mmol, 1 eq) and pyridine (0.300 mL, approximately 5 eq). The resulting solution was heated to reflux for 2 hours. The solution was cooled to room temperature and the solvent was removed under reduced pressure and the residue was chromatographed on silica gel (9:1, hexane: EtOAc) to give 0.180 g (57% yield) of 3-(3,5,5,8,8-pentamethyl-5,6,7,8-tetrahydro-2-naphthyl)-4-methoxybenzaldehyde oxime; mp 152° C.

¹H NMR (500 MHz; CDCl₃): δ[1.27 (s), 1.32 (s), 12 H], 1.69 (s, 4 H), 2.09 (s, 3 H), 3.81 (s, 3 H), 6.96 (d, J=8.3 Hz, 1 H), 7.10 (s, 1 H), 7.14 (s, 1 H), 7.39 (d, J=2.0 Hz, 1 H), 7.56 (q, J=8.3 Hz, J=2.0 Hz, 1 H), 8.11 (s, 1 H).

The intermediate 4-methoxy-3-(3,5,5,8,8-pentamethyl-5,6,7,8-tetrahydronaphthalen-2-yl)benzaldehyde was prepared as follows:

a. (3,5,5,8,8-Pentamethyl-5,6,7,8-tetrahydronaphthalen-2-yl) boronic acid.

The (3,5,5,8,8-pentamethyl-5,6,7,8-tetrahydronaphthalen-2-yl) boronic acid, was prepared in an analogous manner as reported by Dawson et al. (J. Med. Chem. 1995, 38, 3368-3383).

b. 3-(3,5,5,8,8-Pentamethyl-5,6,7,8-tetrahydronaphthalen-2-yl)-4-methoxy-benzaldehyde.

A mixture of 3-bromo-4-methoxybenzaldehyde (19.0 g, 88.4 mmol), (3,5,5,8,8-pentamethyl-5,6,7,8-tetrahydronaphthalen-2-yl) boronic acid (23.8 g, 97.2 mmol) and potassium carbonate (48.8 g, 353.6 mmol) in 1,2-dimethoxyethane (500 mL) and water (40 mL) was degassed with argon for 60 minutes. Tetrakis(triphenylphosphine) palladium(0) (5.0 g, 4.3 mmol) was added and the mixture heated at reflux under argon for 16 hours. The solution was cooled to room temperature, diluted with ethyl acetate (200 mL) and washed successively with water (100 mL) and brine (100 mL), dried over anhydrous magnesium sulfate, filtered and evaporated. The residue was chromatographed on silica gel (eluent: ethyl acetate/hexane, 1:9) to give 26.8 g of 3-(3,5,5,8,8-pentamethyl-5,6,7,8-tetrahydronaphthalen-2-yl)-4-methoxy-benzaldehyde (90%). ¹H NMR (500 MHz; CDCl₃): δ1.26 (s, 6 H); 1.32 (s, 6 H); 1.70 (s, 4 H); 2.08 (s, 3 H); 3.89 (s, 3 H); 7.06 (d, J=8.5 Hz, 1 H); 7.09 (s, 1 H); 7.16 (s, 1 H); 7.71 (d, J=2.0 Hz, 1 H); 7.88 (dd, J₁=2.0 Hz, J₂=8.5 Hz 1 H), 9.91 (s, 1 H).

### Example 2

4-(3,5,5,8,8-Pentamethyl-5,6,7,8-tetrahydro-2-naphthyl)-3-methoxy benzaldehyde.

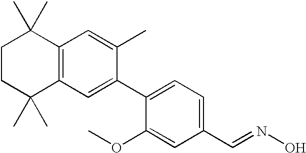

may be prepared in a similar manner to Example 1 using 3-methoxy-4-(3,5,5,8,8-pentamethyl-5,6,7,8-tetrahydro-naphthalen-2-yl)benzaldehyde.

The intermediate 3-methoxy-4-(3,5,5,8,8-pentamethyl-5,6,7,8-tetrahydronaphthalen-2-yl)benzaldehyde was prepared as follows:

a. To a solution of vanillin (1.0 g, 6.57 mmol) in dichloromethane (50 mL) was added pyridine (0.6 mL, 7.76 mmol) and the solution cooled to 0° C. Triflic anhydride (1.3 mL, 7.76 mmol) was added slowly and the reaction mixture warmed slowly to room temperature and stirred overnight at room temperature. The solution was washed successively with water and brine, dried over anhydrous magnesium sulfate, filtered and evaporated. The residue was purified on silica gel (eluent: ethyl acetate/hexane, 1:9) to give 1.38 g of 3-methoxy-4-trifluoromethanesulfonyl benzaldehyde (yield 74%). ¹H NMR (500 MHz; CDCl₃) δ4.00 (s, 3 H); 7.41 (d, J=8.0 Hz, 1 H), 7.50 (dd, J₁=2.0 Hz, J₂=8.0 Hz, 1 H), 7.56 (d, J=2.0 Hz, 1 H), 9.98 (s, 1H).

b. A mixture of 3-methoxy-4-trifluoromethanesulfonyl benzaldehyde (0.50 g, 1.76 mmol), (3,5,5,8,8-pentamethyl-5,6,7,8-tetrahydronaphtalen-2-yl) boronic acid (0.43 g, 1.76 mmol) and potassium carbonate (0.97 g, 7.04 mmol) in 1,2-dimethoxyethane (15 mL) and water (1 mL) was degassed with argon for 30 minutes. Tetrakis(triphenylphosphine)palladium(0) (0.20 g, 0.17 mmol) was added and the mixture heated at reflux under argon for 5 hours. The solution was cooled to room temperature, diluted with ethyl acetate and washed successively with water and brine, dried over anhydrous magnesium sulfate, filtered and evaporated. The residue was chromatographed on silica gel (Biotage, eluent: 0-30% ethyl acetate in hexane) to give 0.40 g of 4-(3,5,5,8,8-pentamethyl-5,6,7,8-tetrahydro-2-naphthyl)-3-methoxybenzaldehyde (67%). ¹H NMR (500 MHz; CDCl₃) δ1.27 (s, 6 H), 1.32 (s, 6 H), 1.70 (s, 4 H), 2.09 (s, 3 H), 3.85 (s, 3 H), 7.09(s, 1 H), 7.16 (s, 1 H), 7.26 (s, 1 H), 7.35 (d, J=7.5 Hz, 1 H); 7.47 (s, 1 H), 7.50 (d, J=7.5 Hz, 1 H), 10.02 (s, 1 H).

### Example 3

2-(3,5,5,8,8-Pentamethyl-5,6,7,8-tetrahydro-2-naphthyl)-5-pyridinecarboxyaldehyde oxime;

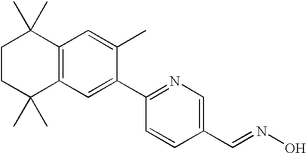

may be prepared in a similar manner to Example 1 using 2-(3,5,5,8,8-pentamethyl-5,6,7,8-tetrahydro-naphthalen-2-yl)pyridine-5-carboxaldehyde.

The intermediate 2-(3,5,5,8,8-pentamethyl-5,6,7,8-tetrahydro-naphthalen-2-yl)pyridine-5-carboxaldehyde was prepared as follows:

a. 2-Bromo-pyridine-5-carboxaldehyde.

To a suspension of 2,5-dibromopyridine (10.28 g, 0.043 mol) in dry ether (150 mL) cooled to −78° C. under argon was added dropwise a solution of n-BuLi (17.4 mL, 0.043 mol, 2.5M in hexanes) while maintaining an internal reaction temperature below −78° C. The resulting dark red suspension was stirred for 30 min. and a solution of DMF (4.0 mL, 0.0521 mol) in 5 mL dry ether was added dropwise. After 45 min. the bath was removed and the mixture was allowed to warm to RT. The mixture was cooled to 0° C. and 1N HCl was added and stirred for 15 min. The resulting layers were separated and the aqueous layer washed with ether (twice) and combined with the original organics. The organics were washed with water, brine and dried (MgSO₄). The mixture was filtered and evaporated to give a solid that was purified by column chromatography (silica gel, CH₂Cl₂) to afford the product as a white solid, 5.23 g (64.8% yield). ¹H NMR (300 MHz; CDCl₃) δ7.69 (d, J=8.0 Hz, 1 H), 8.03 (dd, J₁=8.0 Hz, J₂=2.0 Hz, 1 H), 8.84 (d, J=2.0 Hz, 1 H), 10.10 (s, 1 H).

b. 2-(3,5,5,8,8-Pentamethyl-5,6,7,8-tetrahydro-naphthalen-2-yl)pyridine-5-carboxaldehyde.

A mixture of 2-Bromo-pyridine-5-carboxaldehyde (0.50 g, 2.69 mmol), (3,5,5,8,8-pentamethyl-5,6,7,8-tetrahydronaphtalen-2-yl) boronic acid (0.795 g, 3.23 mmol) and potassium carbonate (0.745 g, 5.38 mmol) in toluene (5mL), EtOH (1 mL) and water (0.75 mL) was degassed with argon for 30 minutes. Tetrakis(triphenyl-phosphine) palladium(0) (0.062 g, 0.054 mmol) was added and the mixture heated at reflux under argon until complete consumption of starting material. The solution was cooled to room temperature, diluted with ethyl acetate and washed successively with water and brine, dried over anhydrous magnesium sulfate, filtered and evaporated. The residue was chromatographed on silica gel (Biotage, eluent: 10% ethyl acetate in hexane) to give 0.744 g of 2-(3,5,5,8,8-pentamethyl-5,6,7,8-tetrahydro-naphthalen-2-yl)pyridine-5-carboxaldehyde (93%).

### Example 4

4-(3,5,5,8,8-Pentamethyl-5,6,7,8-tetrahydro-2-naphthyl)-3-hyrdoxybenzaldehyde oxime;

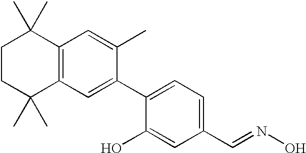

may be prepared in a similar manner to Example 1 using 3-hydroxy-4-(3,5,5,8,8-pentamethyl-5,6,7,8-tetrahydro-naphthalen-2-yl)benzaldehyde.

The intermediate 3-hydroxy-4-(3,5,5,8,8-pentamethyl-5,6,7,8-tetrahydronaphthalen-2-yl)benzaldehyde was prepared as follows:

a. To a solution of 3-methoxy-4-(3,5,5,8,8-pentamethyl-5,6,7,8-tetrahydronaphthalen-2-yl)benzaldehyde (2.0 g, 5.94 mmol, prepared as described in Example 1) in dichloromethane (60 mL) cooled to −78° C. was added BBr₃ (1.12 mL) under argon. The solution was slowly warmed to RT and poured into iced-water. The mixture was extracted with EtOAc, washed with water and brine, dried (MgSO₄), filtered and evaporated to give the crude product. The crude product was taken up into DMF (15 mL) and NaOAc (2.5 g) and the solution was heated to reflux and the temperature maintained overnight. The solution was cooled to RT, diluted with EtOAc and washed successively with water and brine, dried (MgSO₄), filtered and evaporated. The residue was purified on silica gel (eluent: ethyl acetate/hexane, 1:9) to give 1.19 g of 3-hydroxy-4-(3,5,5,8,8-pentamethyl-5,6,7,8-tetrahydro-naphthalen-2-yl) benzaldehyde (yield 62%).

### Example 5

4-(3,5,5,8,8-Pentamethyl-5,6,7,8-tetrahydro-2-naphthyl)-3-ethoxybenzaldehyde oxime;

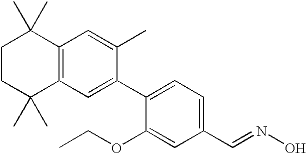

may be prepared in a similar manner to Example 1 using 3-ethoxy-4-(3,5,5,8,8-pentamethyl-5,6,7,8-tetrahydro-naphthalen-2-yl)benzaldehyde.

The intermediate 3-ethoxy-4-(3,5,5,8,8-pentamethyl-5,6,7,8-tetrahydronaphthalen-2-yl)benzaldehyde was prepared in a similar manner as described in Example 2:

a. 3-Ethoxy-4-trifluoromethanesulfonyl benzaldehyde.

To a solution of 4-hydroxy-3-ethoxybenzaldehyde (5.0 g, 30.09 mmol) in dichloromethane (100 mL) was added pyridine (2.92 mL, 36.11 mmol) and the solution cooled to 0° C. Triflic anhydride (6.01 mL, 36.11 mmol) was added slowly and the reaction mixture warmed slowly to room temperature and stirred overnight. The mixture was washed successively with water and brine, dried over anhydrous magnesium sulfate, filtered and evaporated. The residue was purified on silica gel (eluent: ethyl acetate/hexane, 5:95) to give 4.89 g of 3-ethoxy-4-trifluoromethanesulfonyl benzaldehyde (yield 58%).

b. 3-ethoxy-4-(3,5,5,8,8-pentamethyl-5,6,7,8-tetrahydro-naphthalen-2yl)benzaldehyde.

A mixture of 3-methoxy-4-trifluoromethanesulfonyl benzaldehyde (0.51 g, 1.81 mmol), (3,5,5,8,8-pentamethyl-5,6,7,8-tetrahydronaphtalen-2-yl) boronic acid (0.534 g, 2.17 mmol) and potassium carbonate (0.50 g, 3.62 mmol) in toluene (5 mL), EtOH (1 mL) and water (0.75 mL) was degassed with argon for 30 minutes. Tetrakis(triphenyl-phosphine) palladium(0) (0.042 g, 0.036 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was chromatographed on silica gel (Biotage, eluent: 10% ethyl acetate in hexane) to give 0.40 g of 4-(3,5,5,8,8-pentamethyl-5,6,7,8-tetrahydro-2-naphthyl)-3-ethoxybenzaldehyde (67%).

### Example 6

4-(3,5,5,8,8-Pentamethyl-5,6,7,8-tetrahydro-2-naphthyl)-3-methylbenzaldehyde oxime;

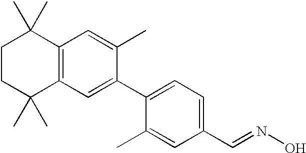

may be prepared in a similar manner as described in Example 1 using the intermediate 3-methyl-4-(3,5,5,8,8-pentamethyl-5,6,7,8-tetrahydro-naphthalen-2-yl) benzaldehyde.

The intermediate 3-methyl-4-(3,5,5,8,8-pentamethyl-5,6,7,8-tetrahydronaphthalen-2-yl)benzaldehyde was prepared in a similar manner as described in Example 2 utilizing 4-hyroxy-3-methylbenzaldehyde; step a) 3-methyl-4-trifluoromethanesulfonyl benzaldehyde (yield 47%) and step b) 3-methyl-4-(3,5,5,8,8-pentamethyl-5,6,7,8-tetrahydro-naphthalen-2-yl)benzaldehyde (yield 83%).

### Example 7

5-(3,5,5,8,8-Pentamethyl-5,6,7,8-tetrahydro-2-naphthyl)-6-methoxy-3-pyridinecarboxaldehyde oxime;

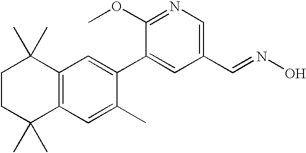

may be prepared in a similar manner as described in Example 1 using 2-methoxy-3-(3,5,5,8,8-pentamethyl-5,6,7,8-tetrahydronaphthalen-2-yl)pyridine-5-carboxaldehyde.

The intermediate 2-methoxy-3-(3,5,5,8,8-pentamethyl-5,6,7,8-tetrahydronaphthalen-2-yl)pyridine-5-carboxaldehyde was prepared as follows:

a. 5-Bromo-2-methoxy-pyridine.

To a suspension of 2-methoxypyridine (10.00 g, 0.09 mol) and sodium acetate (8.27 g, 0.10 mmol) in 30 mL of glacial acetic acid was added a solution of bromine in 20 mL glacial acetic acid while maintaining the reaction temperature below 50° C. After 3 hours, 100 mL of H20 was added and the resulting solution neutralized with cold 2.5M NaOH. The suspension was extracted with ether (2×200 mL), the combined organics were dried over MgSO₄, filtered and evaporated. The crude material was purified on silica gel (eluent:hexane to hexane:ethyl acetate 97:3) and distilled (34-36.5° C./0.05 mm Hg) to give 8.84 g (51.3%) of 5-bromo-2-methoxypyridine as a clear colorless liquid.

b. 2-methoxy-pyridine-5-carboxaldehyde.

To a solution of 5-bromo-2-methoxy-pyridine (8.50 g, 45.2 mmol) in 100 mL dry ether under argon at −64° C. was added 1.6M n-BuLi in hexanes. The resulting mixture was stirred at −64° C. for 40 minutes and allowed to warm to −35° C. To the resulting suspension was added 7.0 mL of dry DMF over 10 minutes. After 15 minutes, the mixture was allowed to warm to 0° C. and 75 mL of 5% NH₄Cl was added. The resulting mixture was separated and the aqueous layer extracted with EtOAc (3×75 mL). The organics were combined, dried (MgSO₄), filtered and evaporated under vacuum to give 2-methoxy-pyridine-5-carboxaldehyde as a tannish solid (recrystallized from hexane), 3.76 g (60.6%); m.p. 48.5-50° C.

c. 2-methoxy-3-bromo-pyridine-5-carboxyaldehyde.

To a suspension of 2-methoxypyridine-5-carboxyaldehyde (3.50 g, 25.5 mmol) and sodium acetate (2.30 g, 28.1 mmol) in 15 mL of glacial acetic acid was added a solution of bromine (1.45 mL, 28.1 mmol) in 20 mL glacial acetic acid and the resulting mixture heated to 100° C. for 18 hours under argon. The mixture was cooled, diluted with water (50 mL) and neutralized with 2.0 M NaOH. The resulting mixture was extracted with ether (4×200 mL), the combined organics dried (MgSO₄), filtered and evaporated. The crude material was purified on silica gel [gradient, hexane:ethyl acetate (99:1) to hexane:ethyl acetate (92:8)] to give 2-methoxy-3-bromo-pyridine-5-carboxyaldehyde as a white solid,0.97 g (17.6%). ¹H NMR (500 MHz, CDCl₃): δ4.11 (s, 3 H), 8.29 (d, J=2.0 Hz, 1 H), 8.56 (d, J=2.0 Hz, 1 H), 9.92 (s, 1 H).

d. 2-Methoxy-3-(3,5,5,8,8-pentamethyl-5,6,7,8-tetrahydronaphthalen-2-yl)pyridine-5-carboxaldehyde.

A mixture of 2-methoxy-3-bromo-pyridine-5-carboxyaldehyde (319 mg, 1.48 mmol), (3,5,5,8,8-pentamethyl-5,6,7,8-tetrahydronaphthalen-2-yl) boronic acid (545 mg, 2.22 mmol), potassium carbonate (817 mg, 5.91 mmol) and water (2 mL) in anhydrous 1,2-dimethoxyethane (30 mL) was degassed with argon for 15 minutes prior to the addition of tetrakis(triphenylphosphine)palladium(0) (342 mg, 0.30 mmol). The reaction mixture was heated under reflux for 15 hours, allowed to cool to room temperature and extracted with ethyl acetate (2×100 mL). The organic extracts were successively washed with water (100 mL), a saturated aqueous solution of NH₄Cl (100 mL), a saturated aqueous solution of NaCl (100 mL), dried over MgSO₄ and filtered. Removal of the solvent under reduced pressure gave an oil which was purified by column chromatography, using a Biotage 12M cartridge, eluting with 5% ethyl acetate/95% hexane. The title compound was isolated in quantitative yield.

¹H NMR (500 MHz, CDCl₃): δ1.24 (s, 6 H), 1.27 (s, 6 H), 1.70 (s, 4 H), 2.09 (s, 3 H), 4.09 (s, 3 H), 7.07 (s, 1 H), 7.17 (s, 1 H), 7.94 (d, J=2.0 Hz, 1 H), 8.64 (d, J=2.0 Hz, 1 H), 10.01 (s, 1 H).

### Example 8

### 3-(3,5,5,8,8-Pentamethyl-5,6,7,8-tetrahydro-2-naphthyl)-2-fluoro-4-methoxybenzaldehyde oxime;

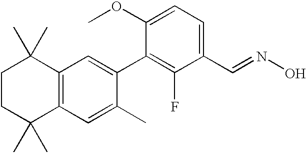

may be prepared in a similar manner as described in Example 1 in a 57% yield using 3-(3,5,5,8,8-Pentamethyl-5,6,7,8-tetrahydro-2-naphthyl)-2-fluoro-4-methoxybenzaldehyde.

The intermediate 3-(3,5,5,8,8-pentamethyl-5,6,7,8-tetrahydro-2-naphthyl)-2-fluoro-4-methoxybenzaldehyde was prepared as follows:

a. 2-Fluoro-3-hydroxy-4-methoxybenzaldehyde.

To a heated solution (80° C.) of hexamethylenetetramine (2.8 g, 20 mmol) in trifluoroacetic acid (10 mL) was added dropwise over a 50 minutes period 2-fluoro-6-methoxyphenol (1.42 g, 10 mmol) in trifluoroacetic acid (10 mL). The mixture was heated for an additional 1 hour, concentrated and water (50 ml) was added. The solution was stirred for 10 minutes and solid potassium carbonate was added until the solution was neutral. The solid that formed was collected to afford 1.1 g of 2-fluoro-3-hydroxy-4-methoxybenzaldehyde (65%). ¹H NMR (500 MHz; DMSO-d₆): δ3.90 (s, 3 H); 6.98 (d, J=8.5 Hz, 1 H); 7.31 (t, J=8.5 Hz, 1 H); 9.66 (br, 1 H); 10.02 (s, 1 H).

b. 2-Fluoro-4-methoxy-3-trifluoromethanesulfonyl benzaldehyde.

To a solution of 2-fluoro-3-hydroxy-4-methoxybenzaldehyde (1.1 g, 6.47 mmol) in dichloromethane (50 mL) was added pyridine (0.6 mL, 7.76 mmol) and the solution cooled to 0° C. Triflic anhydride (1.3 mL, 7.76 mmol) was added slowly and the reaction mixture warmed slowly to room temperature and stirred overnight at room temperature. The solution was washed successively with water and brine, dried over anhydrous magnesium sulfate, filtered and evaporated. The residue was chromatographed and silica gel (eluent: ethyl acetate/hexane, 1:4) to give 1.21 g of 2-fluoro-4-methoxy-3-trifluoromethanesulfonyl benzaldehyde (62%). ¹H NMR (500 MHz; CDCl₃): δ4.03 (s, 3 H), 6.95 (d, J=8.0 Hz, 1 H), 7.89 (dd, J₁=8.0 Hz, J₂=9.0 Hz, 1 H), 10.20 (s, 1 H).

c. 2-Fluoro-4-methoxy-3-trifluoromethanesulfonyl benzaldehyde.

A mixture of 2-fluoro-4-methoxy-3-trifluoromethanesulfonyl benzaldehyde (1.21 g, 4.01 mmol), (3,5,5,8,8-pentamethyl-5,6,7,8-tetrahydronaphthalen-2-yl) boronic acid (1.08 g, 4.41 mmol) and potassium carbonate (2.22 g, 16.04 mmol) in 1,2-dimethoxyethane (30 mL) and water (2 mL) was degassed with argon for 30 minutes. Tetrakis(triphenylphosphine)palladium(0) (0.23 g, 0.2 mmol) was added and the mixture heated at reflux under argon for 16 hours. The solution was cooled to room temperature, diluted with ethyl acetate and washed successively with water and brine, dried over anhydrous magnesium sulfate, filtered and evaporated. The residue was chromatographed on silica gel (Biotage, eluent: ethyl acetate/hexane, 0.5:8.5) to give 0.87 g of 4-methoxy-2-fluoro-3-(3,5,5,8,8-pentamethyl-5,6,7,8-tetrahydronaphthalen-2-yl) benzaldehyde (62%). ¹H NMR (500 MHz; CDCl₃): δ1.26 (s, 6 H), 1.32 (s, 6 H), 1.69 (s, 4 H), 2.07 (s, 3 H), 3.85 (s, 3 H), 7.07 (d, J=8.8 Hz, 1 H), 7.07 (s, 1 H), 7.19 (s, 1 H), 7.90 (t, J=8.8 Hz, 1 H), 10.25 (s, 1 H).

### Example 9

6-(3,5,5,8,8-Pentamethyl-5,6,7,8-tetrahydro-2-naphthyl)-5-methoxy-2-pyridinecarboxaldehyde oxime;

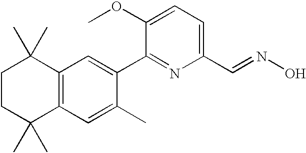

may be prepared in a similar manner as described in Example 1 using 6-(3,5,5,8,8-pentamethyl-5,6,7,8-tetrahydronaphthalen-2-yl)-5-methoxy-pyridine-2-carboxaldehyde.

The intermediate 6-(3,5,5,8,8-Pentamethyl-5,6,7,8-tetrahydronaphth-2-yl)-5-methoxy-pyridine-2-carboxaldehyde was prepared as follows:

a. 2-Bromo-3-hydroxy-6-methyl-pyridine.

To a solution of 5-hydroxy-2-methylpyridine (8.80 g, 80.6 mmol) in 125 mL of pyridine was added a solution of bromine (14.18 g, 88.7 mmol) in 50 mL pyridine dropwise. The temperature of the reaction mixture rose to 40° C. upon completion of addition. After 1 hour the pyridine was removed under vacuum and the resulting solid was suspended into water (200 mL) and stirred overnight. The solid was collected and dried to give the desire product as a brownish solid (8.05 g, 53.1% yield). ¹H NMR (500 MHz, CDCl₃): δ2.21 (s, 3 H), 6.73 (d, J=8.1 Hz, 1 H), 6.94 (d, J=8.1 Hz, 1 H), 9.36 (brs, 1 H).

b. 2-Bromo-3-methoxy-6-methyl-pyridine.

A stirred mixture of 2-bromo-3-hydroxy-6-methyl-pyridine (7.89 g, 42.0 mmol), potassium carbonate (11.60 g, 83.9 mmol) and iodomethane (8.93 g, 62.9 mmol, 3.92 mL) in acetone (100 mL) was heated under reflux overnight. The mixture was filtered, evaporated and purified on silica gel (hexane:ethyl acetate, 95:5 to 9:1) to give the desired product as a white solid (7.49 g, 88.3%). ¹H NMR (500 MHz, CDCl₃): δ2.46 (s, 3 H), 3.87 (s, 3 H), 7.04 (s, 2 H).

c. 5-Methoxy-6-bromo-pyridine-2-carboxaldehyde.

A stirred mixture of 2-bromo-3-methoxy-6-methyl-pyridine (2.00 g, 9.9 mmol), Cu(II) sulfate pentahydrate (2.47 g, 9.9 mmol), and potassium peroxydisulfate (8.03 g, 29.7 mmol) in 80 mL of acetonitrile/H₂O (1:1) was heated under reflux. After 1 hour, the dark green mixture was cooled to room temperature and CH₂Cl₂ was added. The layers were separated and the aqueous layer further extracted with CH₂Cl₂. The organics were combined, dried (MgSO₄), filtered and evaporated. The resulting crude product was purified on silica gel [Biotage, hexane:ethyl acetate (4:1)] to give a white solid (0.51 g, 24% yield).

d. 6-(3,5,5,8,8-Pentamethyl-5,6,7,8-tetrahydronaphth-2-yl)-5-methoxypyridine-2-carboxaldehyde.

A mixture of 6-bromo-5-methoxypyridine-2-carboxaldehyde (512 mg, 2.37 mmol), (3,5,5,8,8-pentamethyl-5,6,7,8-tetrahydronaphthalen-2-yl) boronic acid (700 mg, 2.84 mmol) and potassium carbonate (1.31 g, 9.5 mmol) in 1,2-dimethoxyethane (22 mL) and water (2 mL) was degassed with argon. Tetrakis(triphenylphosphine) palladium(0) (550 mg, 0.48 mmol) was added and the mixture heated under reflux under argon for 24 hours. The solution was cooled to room temperature, diluted with ethyl acetate and washed successively with water and brine, dried over anhydrous magnesium sulfate, filtered and evaporated. The residue was chromatographed on silica gel (Biotage hexane:EtOAc 9:1) to give 603 mg (75% yield) of 6-(3,5,5,8,8-pentamethyl-5,6,7,8-tetrahydronaphth-2-yl)-5-methoxy-pyridine-2-carboxaldehyde.

¹H NMR (500 MHz; CDCl₃): δ1.29 (s, 6 H), 1.31 (s, 6 H), 1.70 (s, 4 H), 2.15 (s, 3 H), 3.90 (s, 3 H), 7.20 (s, 1 H), 7.27 (s, 1 H), 7.37 (d, J=8.5 Hz, 1 H), 8.00 (d, J=8.5 Hz, 1 H), 10.04 (s, 1 H).

### Example 10

3-(1,4-Diisopropyl-6-methyl-1,2,3,4-tetrahydro-7-quinoxalinyl)-4-methoxybenzaldehyde oxime;

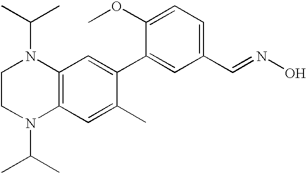

may be prepared in a similar manner as described in Example 1 using 3-(1,4-diisopropyl-6-methyl-1,2,3,4-tetrahydro-7-quinoxalinyl)-4-methoxybenzaldehyde.

The intermediate 3-(1,4-diisopropyl-6-methyl-1,2,3,4-tetrahydro-7-quinoxalinyl)-4-methoxybenzaldehyde was prepared as follows:

a. 6-Methyl-1,2,3,4-tetrahydroquinoxaline.

To a solution of 6-methylquinoxaline (2 g, 13.87 mmol) and nickel (II) chloride hexahydrate (6.6 g, 27.74 mmol) in anhydrous methanol (70 mL) was added in portions, sodium borohydride (10.5 g, 277.43 mmol) while maintaining the temperature between 0° C. and 5° C. The reaction mixture was stirred at 0° C. for 20 minutes and at room temperature for 4 hours. Removal of the solvent under reduced pressure was ensued by acidification of the residue with 2N HCl (600 mL). The mixture was stirred at room temperature for 16 hours and filtered. The green filtrate was made basic (pH 10-11) using concentrated NH₄OH (150 mL) and extracted with diethylether (3×200 mL). The ethereal extracts were successively washed with water (2×300 mL), a saturated aqueous solution of NaCl (150 mL), dried over MgSO₄ and filtered. Removal of the solvent under reduced pressure gave 6-methyl-1,2,3,4-tetrahydroquinoxaline as a solid (880 mg, 43%).

¹H NMR (500 MHz; CDCl₃): δ2.17 (s, 3 H), 3.39-3.40 (m, 4 H), 6.41-6.33 (m, 3 H).

b. 1,4-diisopropyl-6-methyl-1,2,3,4-tetrahydroquinoxaline.

A mixture of 6-methyl-1,2,3,4-tetrahydroquinoxaline (851 mg, 5.75 mmol), potassium carbonate (3.18 g, 23 mmol) and 2-iodopropane (4.6 mL, 46 mmol) in dry dimethylformamide (10 mL) was heated under reflux for 19 hours, allowed to cool to room temperature prior to the addition of water (100 mL) and extracted with ethyl acetate (2×75 mL). The organic extracts were successively washed with a saturated aqueous solution of NH₄Cl (100 mL), a saturated aqueous solution of NaCl (100 mL), dried over MgSO₄ and filtered. Removal of the solvent under reduced pressure gave a dark orange oil which was purified by column chromatography, using a Biotage 40S cartridge, eluting with 5% ethyl acetate/95% hexane, to give 1,4-diisopropyl-6-methyl-1,2,3,4-tetrahydroquinoxaline as a solid (870 mg, 66%).

¹H NMR (500 MHz; CDCl₃): δ1.19-1.16 (m, 12 H), 2.24 (s, 3 H), 3.16-3.14 (m, 2 H), 3.23-3.21 (m, 2 H), 4.02 (quintet, J=6.5 Hz, 1 H), 4.08 (quintet, J=6.5 Hz, 1 H), 6.44 (d, J=8.0 Hz, 1 H), 6.49 (s, 1 H), 6.56 (d, J=8.1 Hz, 1 H).

c. 7-Bromo-1,4-diisopropyl-6-methyl-1,2,3,4-tetrahydroquinoxaline.

A mixture of 1,4-diisopropyl-6-methyl-1,2,3,4-tetrahydroquinoxaline (516 mg, 2.22 mmol) and tetrabutylammonium tribromide (1.18 g, 2.45 mmol) in anhydrous dichloromethane (20 mL) was stirred at room temperature for 4 hours. The solution was washed successively with a saturated aqueous solution of NaHCO₃ (150 mL), water (150 mL), a saturated aqueous solution of NaCl (150 mL), dried over MgSO₄ and filtered. Removal of the solvent under reduced pressure gave a solid which was purified by column chromatography, using a Biotage 40S cartridge, eluting with 5% ethyl acetate/95% hexane, to give 7-bromo-1,4-diisopropyl-6-methyl-1,2,3,4-tetrahydroquinoxaline as a white solid (480 mg, 70%).

¹H NMR (500 MHz; CDCl₃): δ1.16-1.15 (m, 12 H), 2.25 (s, 3 H), 3.16 (s, 4 H), 3.95 (quintet, J=6.6 Hz, 1 H), 4.00 (quintet, J=6.6 Hz, 1 H), 6.47 (s, 1 H), 6.73 (s, 1 H).

d. 3-(1,4-Diisopropyl-6-methyl-1,2,3,4-tetrahydro-7-quinoxalinyl)-4-methoxybenzaldehyde.

A mixture of 7-bromo-1,4-diisopropyl-6-methyl-1,2,3,4-tetrahydroquinoxaline (469 mg, 1.51 mmol), 2-methoxy-5-formylphenylboronic acid (407 mg, 2.26 mmol), potassium carbonate (834 mg, 6.03 mmol) and water (2.5 mL) in anhydrous 1,2-dimethoxyethane (30 mL) was degassed with argon for 15 minutes prior to the addition of tetrakis(triphenylphosphine)palladium(0) (349 mg, 0.30 mmol). The reaction mixture was heated under reflux for 8.5 hours, allowed to cool to room temperature and extracted with ethyl acetate (2×100 mL). The organic extracts were successively washed with water (100 mL), saturated aqueous NH₄Cl (100 mL), saturated aqueous NaCl (100 mL), dried over MgSO₄ and filtered. Removal of the solvent under reduced pressure gave an oil which was purified by column chromatography, using a Biotage 40S cartridge, eluting with 10% ethyl acetate/90% hexane. The title compound was isolated as bright yellow solid (315 mg, 57%).

¹H NMR (500 MHz; CDCl₃): δ1.14 (d, J=6.6 Hz, 6 H), 1.20 (d, J=6.8 Hz, 6 H), 2.01 (s, 3 H), 3.19-3.17 (m, 2 H), 3.27-3.25 (m, 2 H), 3.86 (s, 3 H), 3.99 (quintet, J=6.6 Hz, 1 H), 4.11 (quintet, J=6.6 Hz, 1 H), 6.47 (s, 1 H), 6.51 (s, 1 H), 7.03 (d, J=8.7 Hz, 1 H), 7.72 (d, J=1.9 Hz, 1 H), 7.84 (dd, J=8.3 Hz, J=2.0 Hz, 1 H), 9.90 (s, 1 H).

### Example 11

3-(3,5,5,8,8-Pentamethyl-5,6,7,8-tetrahydro-2-naphthyl)-4-methoxy-6-hydroxybenzaldehyde oxime;

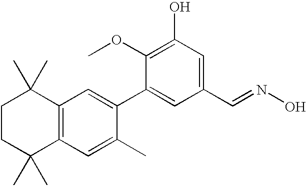

may be prepared in a similar manner as described in Example 1 using 3-(3,5,5,8,8-pentamethyl-5,6,7,8-tetrahydro-2-naphthyl)-4-methoxy-6-hydroxybenzaldehyde.

The intermediate 3-(3,5,5,8,8-pentamethyl-5,6,7,8-tetrahydro-2-naphthyl)-4-methoxy-6-hydroxybenzaldehyde was prepared as follows:

a. 3-Bromo-6-hydroxy-4-methoxy-benzaldehyde.

A mixture of 2-hydroxy-4-methoxy-benzaldehyde (3.04 g, 20 mmol) and tetrabutylammonium tribromide (6.40 g, 20 mmol) in anhydrous dichloromethane (200 mL) was stirred at room temperature for 24 hours. The solution was washed successively with a saturated aqueous solution of NaHCO₃ (150 mL), water (150 mL), a saturated aqueous solution of NaCl (150 mL), dried over MgSO₄ and filtered. Removal of the solvent under reduced pressure gave a solid which was purified by column chromatography, using a Biotage 40M cartridge, eluting with 5% ethyl acetate/95% hexane to give 3-bromo-6-hydroxy-4-methoxy-benzaldehyde as a white solid (3.50 g, 76%).

¹H NMR (500 MHz; CDCl₃): δ3.94 (s, 3 H), 6.47 (s, 1 H), 7.67 (s, 1 H), 9.68 (s, 1 H), 11.43 (s, 1 H).

b. 3-(3,5,5,8,8-pentamethyl-5,6,7,8-tetrahydro-2-naphthyl)-4-methoxy-6-hydroxybenzaldehyde.

A mixture of 3-bromo-6-hydroxy-4-methoxy-benzaldehyde (2 g, 8.66 mmol), (3,5,5,8,8-pentamethyl-5,6,7,8-tetrahydronaphthalen-2-yl) boronic acid (3.18 g, 12.99 mmol), potassium carbonate (4.79 g, 34.63 mmol) and water (4 mL) in anhydrous 1,2-dimethoxyethane (140 mL) was degassed with argon for 15 minutes prior to the addition of tetrakis(triphenylphosphine)palladium(0) (2.0 g, 1.73 mmol). The reaction mixture was heated under reflux for 15 hours, allowed to cool to room temperature and extracted with ethyl acetate (2×100 mL). The organic extracts were successively washed with water (100 mL), a saturated aqueous solution of NH₄Cl (100 mL), a saturated aqueous solution of NaCl (100 mL), dried over MgSO₄ and filtered. Removal of the solvent under reduced pressure gave an oil which was purified by column chromatography, using a Biotage 40M cartridge, eluting with 5% ethyl acetate/95% hexane, to give 3-(3,5,5,8,8-pentamethyl-5,6,7,8-tetrahydro-2-naphthyl)-4-methoxy-6-hydroxybenzaldehyde as a white solid (2.2 g, 73%).

¹H NMR (500 MHz; CDCl₃): δ1.28 (s, 6 H), 1.33 (s, 6 H), 1.70 (s, 4 H), 2.08 (s, 3 H), 3.84 (s, 3 H), 6.51 (s, 1 H), 7.07 (s, 1 H), 7.15 (s, 1 H), 7.31 (s, 1 H), 9.73 (s, 1 H), 11.53 (s, 1 H).

### Example 12

3-(3,5,5,8,8-Pentamethyl-5,6,7,8-tetrahydro-2-naphthyl)-4,6-dimethoxybenzaldehyde oxime;

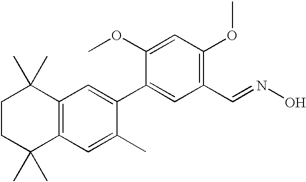

may be prepared in a similar manner as described in Example 1 using 3-(3,5,5,8,8-pentamethyl-5,6,7,8-tetrahydro-2-naphthyl)-4,6-dimethoxybenzaldehyde.

The intermediate 3-(3,5,5,8,8-pentamethyl-5,6,7,8-tetrahydro-2-naphthyl)-4,6-dimethoxybenzaldehyde was prepared as follows:

To a solution of 3-(3,5,5,8,8-pentamethyl-5,6,7,8-tetrahydro-2-naphthyl)-4-methoxy-6-hydroxybenzaldehyde (1.04 g, 2.95 mmol) in acetone (20 mL) was added dimethylsulfate (0.37 mL, 3.84 mmol) and potassium carbonate (490 mg, 3.55 mmol). The reaction mixture was stirred at room temperature for 15 hours and extracted with ethyl acetate (2×50 mL). The organic extracts were successively washed with water (100 mL) and a saturated aqueous solution of NaCl (100 mL), dried over MgSO₄ and filtered. Removal of the solvent under reduced pressure gave 3-(3,5,5,8,8-pentamethyl-5,6,7,8-tetrahydro-2-naphthyl)-4,6-dimethoxybenzaldehyde (1.05 g, 97%).

¹H NMR (500 MHz; CDCl₃): δ1.26 (s, 6 H), 1.31 (s, 6 H), 1.69 (s, 4 H), 2.06 (s, 3 H), 3.87 (s, 3 H), 3.99 (s, 3 H), 6.50 (s, 1 H), 7.05 (s, 1 H), 7.13 (s, 1 H), 7.67 (s, 1 H), 10.35 (s, 1 H).

### Example 13

3-(5,5,8,8-Tetramethyl-5,6,7,8-tetrahydro-2-naphthyl)-2,4-dimethoxybenzaldehyde oxime;

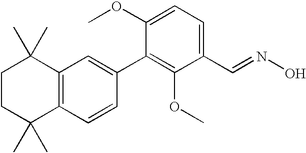

may be prepared in a similar manner as described in Example 1 using 3-(5,5,8,8-tetramethyl-5,6,7,8-tetrahydronaphthylen-2-yl)-2,4-dimethoxy-benzaldehyde.

The intermediate 3-(5,5,8,8-tetramethyl-5,6,7,8-tetrahydro-2-naphthyl)-2,4-dimethoxy-benzaldehyde was prepared as follows:

a. 6-(2,6-Dimethoxyphenyl)-1,1,4,4-tetramethy-1,2,3,4-tetrahydronaphthlene.

A mixture of 2,6-dimethoxy-phenylboronic acid (1.0 g, 5.48 mmol), 6-bromo-1,1,4,4 tetramethyl 1,2,3,4-tetrahydronaphthalene (0.73 g, 2.74 mmol) and potassium carbonate (1.50 g, 10.96 mmol) in 1,2-dimethoxyethane (20 mL) and water (1.0 mL) was degassed with argon for 15 minutes. Tetrakis(triphenylphosphine)palladium(0) (0.60 g, 0.54 mmol) was added and the mixture heated at reflux under argon for 5 hours. The solution was cooled to room temperature, diluted with ethyl acetate and washed successively with water and brine, dried over anhydrous magnesium sulfate, filtered and evaporated. The residue was chromatographed on silica gel (eluent: ethyl acetate/hexane, 1:9) to give 0.92 g of 6-(2,6-Dimethoxyphenyl)-1,1,4,4-tetramethy-1,2,3,4-tetrahydronaphthlene.

b. 6-(5-Bromo-2,6-dimethoxyphenyl)-1,1,4,4-tetramethy-1,2,3,4-tetrahydronaphthlene.

To a solution of 6-(2,6-dimethoxyphenyl)-1,1,4,4-tetramethy-1,2,3,4-tetrahydronaphthlene (340 mg, 1.05 mmol) in dichloromethane (10 mL) was added pyridinium tribromide (335 mg, 1.05 mmol) and the reaction mixture stirred at room temperature overnight. The solution was diluted with ethyl acetate and washed successively with water and brine, dried over anhydrous magnesium sulfate, filtered and evaporated. The residue was purified on silica gel (eluent: ethyl acetate/hexane, 0.5:9.5) to give 0.24 g (57%) of 6-(5-bromo-2,6-dimethoxyphenyl)-1,1,4,4-tetramethy-1,2,3,4-tetrahydronaphthlene. ¹H NMR (500 MHz; CDCl₃): δ1.28 (s, 6 H); 1.31 (s, 6 H); 1.70 (s, 4 H); 3.35 (s, 3 H); 3.73 (s, 3 H); 7.14 (dd, J₁=1.5 Hz, J₂=8.5 Hz, 1 H); 7.15 (dd, J₁=2.0 Hz, J₂=8.5 Hz, 1H); 7.30 (d, J=8.0 Hz, 1H); 7.36 (d, J=1.5 Hz, 1H); 7.45 (d,

J=8.0 Hz, 1 H), 7.95 (br, 1 H).

c. 3-(5,5,8,8-Tetramethyl-5,6,7,8-tetrahydro-2-naphthyl)-2,4-dimethoxybenzaldehyde.

To a solution of 6-(5-bromo-2,6-dimethoxyphenyl)-1,1,4,4-tetramethy-1,2,3,4-tetrahydronaphthlene (0.24 g, 0.55 mmol) in anhydrous THF (6 mL) was added at −78° C. under argon n-BuLi (1.6M in hexane, 0.7 mL, 1.1 mmol). The solution was stirred for 5 minutes at −78° C. and N,N-dimethylformamide (0.13 mL, 1.65 mmol) was added. The reaction mixture was stirred 2 hours at −78° C. then quenched with aqueous ammonium chloride and brought to room temperature. The solution was diluted with ethyl acetate and washed successively with water and brine, dried over anhydrous magnesium sulfate, filtered and evaporated. The residue was chromatographed and silica gel (eluent: ethyl acetate/hexane, 1:9) to give 0.14 g (72%) of 3-(5,5,8,8-tetramethyl-5,6,7,8-tetrahydro-2-naphthyl)-2,4-dimethoxy-benzaldehyde. ¹H NMR (500 MHz; CDCl₃): δ1.29 (s, 6 H); 1.32 (s, 6 H); 1.72 (s, 4 H); 3.37 (s, 3 H); 3.83 (s, 3 H); 6.83 (d, J=9.0 Hz, 1 H); 7.14 (dd, J₁=2.0 Hz, J₂=8.5 Hz, 1 H); 7.33 (d, J=8.0 Hz, 1 H); 7.36 (s, 1 H); 7.85 (d, J=8.5 Hz, 1 H); 10.29 (s, 1 H).

### Example 14

3-(1-Isopropyl-7-methyl-1,2,3,4-tetrahydro-6-quinolinyl)-4-methoxybenzaldehyde oxime;

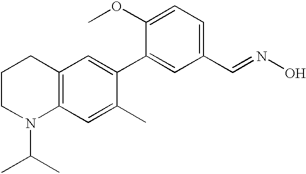

may be prepared in a similar manner as described in Example 1 using 3-(1-Isopropyl-7-methyl-1,2,3,4-tetrahydro-6-quinolinyl)-4-methoxybenzaldehyde.

The intermediate 3-(1-Isopropyl-7-methyl-1,2,3,4-tetrahydro-6-quinolinyl)-4-methoxybenzaldehyde was prepared as follows:

a. 1-Isopropyl-7-methyl-1,2,3,4-tetrahydro-6-bromoquinoline.

To a cooled solution of 7-methyl quinoline (5.00 g, 35 mmol) and nickel (II) chloride hexahydrate (1.40 g, 6 mmol) in methanol (130 mL) was added sodium borohydride (5.50 g, 140 mmol) portionwise. The reaction mixture was stirred at 0° C. for 1 hour and then at room temperature for 3 hours. Hydrochloric acid (2N, 200 mL) was added to the black residue and the mixture stirred at room temperature until disappearance of the black precipitate. The acidic solution was neutralized with concentrated ammonium hydroxide and extracted with ether. The organic layer was washed with brine and dried over anhydrous magnesium sulfate, filtered and evaporated to give 5.28 g of 7-methyl-1,2,3,4-tetrahydro-quinoline (100%), used without further purification. A mixture of 7-methyl-1,2,3,4-tetrahydro-quinoline (1.20 g, 8.2 mmol), potassium carbonate (2.3 g, 16.4 mmol) and isopropyl iodide (3.3 mL, 32.8 mmol) in N,N-dimethylformamide (10 mL) was heated at 60° C. with stirring for 24 hours. The solution was cooled to room temperature and washed successively with water and brine, dried over anhydrous magnesium sulfate, filtered and evaporated to give 1.28 g (82%) of 1-isopropyl-7-methyl-1,2,3,4-tetrahydro-quinoline. To a solution of 1-isopropyl-7-methyl-1,2,3,4-tetrahydro-quinoline (1.04 g, 5.5 mmol) in dichloromethane was added tetrabutylammonium tribromide (2.65 g, 5.5 mmol) and the solution stirred at room temperature for 5 hours. The solution was washed successively with water and brine, dried over anhydrous magnesium sulfate, filtered and evaporated. The residue was chromatographed and silica gel (ethyl acetate/hexane, 1:9) to give 1.00 g of 6-bromo-1-isopropyl-7-methyl-1,2,3,4-tetrahydro-quinoline (67%).

¹H NMR(500 MHz; CDCl₃): δ1.10 (s, 3 H); 1.11 (s, 3 H); 1.81 (m, 2 H); 2.20 (s, 3 H); 2.64 (m, 2 H); 3.0.8 (m, 2 H); 3.5 (m, 1 H); 6.94 (s, 1 H); 6.54 (s, 1 H); 7.08 (s, 1 H).

b. 3-(1-Isopropyl-7-methyl-1,2,3,4-tetrahydro-6-quinolinyl)-4-methoxybenzaldehyde.

A mixture of 6-bromo-1-isopropyl-7-methyl-1,2,3,4-tetrahydro-quinoline (0.85 g, 3.16 mmol), 2-methoxy-5-formyl boronic acid (1.13 g, 6.31 mmol) and potassium carbonate (1.70 g, 12.64 mmol) in 1,2-dimethoxyethane (30 mL) and water (1.5 mL) was degassed with argon for 15 minutes. Tetrakis(triphenylphosphine)palladium(0) (0.80 g, 0.63 mmol) was added and the mixture heated at reflux under argon for 35 hours. The solution was cooled to room temperature, diluted with ethyl acetate and washed successively with water and brine, dried over anhydrous magnesium sulfate, filtered and evaporated. The residue was chromatographed and silica gel (eluent: ethyl acetate/hexane, 1:9) to give 0.81 g of 3-(1-isopropyl-7-methyl-1,2,3,4-tetrahydro-6-quinolinyl)-4-methoxybenzaldehyde (79%). ¹H NMR (500 MHz; CDCl₃): δ1.18 (s, 3 H); 1.20 (s, 3 H); 1.94 (m, 2 H); 2.06 (s, 3 H); 2.72 (m, 2 H); 3.18 (m, 2 H); 3.85 (s, 3 H); 4.16 (m, 1 H); 6.57 (s, 1 H); 6.78 (s, 1 H); 7.02 (d, 1 H); 7.69 (d, 1 H); 7.34 (s, 1 H); 7.84 (m, 1 H); 9.89 (s, 1 H).

### Example 15

3-(3,5,5,8,8-Pentamethyl-5,6,7,8-tetrahydro-2-naphthyl)-4,5-dimethoxybenzaldehyde oxime;

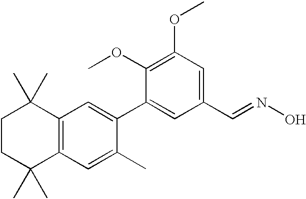

may be prepared in a similar manner as described in Example 1 using 3-(3,5,5,8,8-pentamethyl-5,6,7,8-tetrahydro-2-naphthyl)-4,5-dimethoxybenzaldehyde prepared from 3-bromo-4,5-dimethoxybenzaldehyde.

The intermediate 3-bromo-4,5-dimethoxybenzaldehyde was prepared as follows:

To a solution of 5-bromovanillin (2.00 g, 8.65 mmol) in acetone (50 mL) was added potassium carbonate (1.4 g, 10.38 mmol) and dimethylsulfate (1 mL, 10.38 mmol). The solution was stirred at room temperature for 16 hours. The reaction mixture was diluted with ethyl acetate and the organic layer was washed successively with water and brine, dried over anhydrous magnesium sulfate, filtered and evaporated to afford 1.88 g of 3-bromo-4,5-dimethoxybenzaldehyde (89%).

### Example 16

3-(3,5,5,8,8-Pentamethyl-5,6,7,8-tetrahydro-2-naphthyl)-4-hydroxybenzaldehyde oxime;

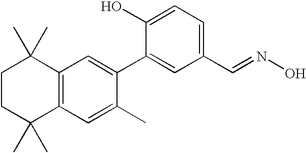

may be repaired in a similar manner as described in Example 1 using 4-hydroxy-3-(3,5,5,8,8-pentamethyl-5,6,7,8-tetrahydro-naphthalen-2-yl)benzaldehyde.

The intermediate 4-hydroxy-3-(3,5,5,8,8-pentamethyl-5,6,7,8-tetrahydronaphthalen-2-yl)benzaldehyde was prepared as follows:

To a solution of 4-methoxy-3-(3,5,5,8,8-pentamethyl-5,6,7,8-tetrahydro-naphthalen-2-yl)benzaldehyde (0.30 g, 0.89 mmol) in anhydrous dichloromethane (10 ml) at −78° C. under argon was added boron tribromide (0.17 mL, 1.78 mmol). The solution was slowly warmed to room temperature and stirred for 24 hrs. The solution was carefully poured onto ice water and extracted with ethyl acetate. The organic layer was further washed with water and brine, dried over anhydrous magnesium sulfate, filtered and evaporated. The residue was chromatographed on silica gel (Biotage, eluent: ethyl acetate/hexane, 1:9) to give 0.24 g of product (84%). ¹H NMR (500 MHz; CDCl₃) δ1.26 (s, 6 H), 1.32 (s, 6,H), 1.71 (s, 4 H), 2.10 (s, 3 H), 5.46 (s, 1 H), 7.11 (d, J=8.3 Hz, 1 H), 7.13 (s, 1 H); 7.26 (s, 1 H); 7.69 (d, J=1.8 Hz, 1 H); 7.83 (dd, J₁=6.8 Hz, J₂=1.8 Hz, 1 H), 9.89 (s, 1 H).

### Example 17

3-(3,5-Di-t-butyl-4-hydroxyphenyl)-4-methoxybenzaldehyde oxime;

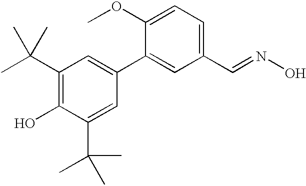

may be prepared in a similar manner as described in Example 1 using 3-(3,5-di-t-butyl-4-hydroxyphenyl)-4-methoxybenzaldehyde.

The intermediate 3-(3,5-di-t-butyl-4-hydroxyphenyl)-4-methoxybenzaldehyde was prepared in a manner similar to the procedure described in Example 1 b using 4-bromo-2,6-di-t-butylphenol (0.50 g, 1.75 mmol), 2-methoxy-5-formylphenyl boronic acid (0.315 g, 1.75 mmol), tetrakis(triphenylphosphine)palladium(0) (0.20 g, 0.175 mmol), K₂CO₃ (0.95 g, 7.0 mmol), dimethoxyethane (15 mL) and H₂O (1 mL); 367 mg, 61% yield.

¹H NMR (500 MHz; CDCl₃) δ1.48 (s, 18 H), 3.93 (s, 3 H), 5.30 (s, 1 H), 7.08 (d, J=8.0 Hz, 1 H), 7.36 (s, 2 H), 7.80-7.85 (m, 2 H), 9.94 (s, 1 H).

### Example 18

3-(3,5,5,8,8-Pentamethyl-5,6,7,8-tetrahydro-2-naphthyl)-4-dimethylaminobenzaldehyde;

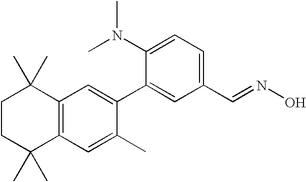

may be prepared in a similar manner as described in Example 1 using 4-dimethylamino-3-(3,5,5,8,8-pentamethyl-5,6,7,8-tetrahydronaphtalen-2-yl)benzaldehyde.

The intermediate 4-dimethylamino-3-(3,5,5,8,8-pentamethyl-5,6,7,8-tetrahydronaphtalen-2-yl)benzaldehyde was prepared as follows:

a. 3-Bromo-4-(dimethylamino)-benzaldehyde.

To a solution of 4-(dimethylamino)-benzaldehyde (10.0 g, 67.03 mmol) in dichloromethane (250 mL) was added pyridinium tribromide (21.4 g, 67.03 mmol). The reaction mixture was stirred at room temperature overnight. The solution was washed successively with water and brine, dried over anhydrous magnesium sulfate, filtered and evaporated. Choromatography on silica gel (15% EtOAc in hexane) afforded 14.06 g of 3-bromo-4-(dimethylamino)-benzaldehyde (92%).

b. 4-Dimethylamino-3-(3,5,5,8,7-pentamethyl-5,6,7,8-tetrahydronaphthalen-2-yl) benzaldehyde.

To a solution of 3-bromo-4-(dimethylamino)-benzaldehyde (5 g, 21.92 mmol), (3,5,5,8,8-pentamethyl-5,6,7,8-tetrahydronaphtalen-2-yl) boronic acid (6.5 g, 26.30 mmol) in a mixture of toluene (50 mL), ethanol (10 mL) and water (7.5 mL) was added potassium carbonate (6.0 g, 43.83 mmol). The solution was degased with argon for 30 minutes. Tetrakis(triphenylphosphine)palladium(0) (0.50 g, 0.438 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was chromatographed on silica gel (8% ethyl acetate in hexane) to give 7.08 g of 4-dimethylamino-3-(3,5,5,8,7-pentamethyl-5,6,7,8-tetrahydronaphtalen-2-yl) benzaldehyde (92%). ¹H NMR (300 MHz; DMSO) δ1.22 (s, 3H); 1.28 (s, 3H); 1.29 (s, 3H); 1.31 (s, 3H); 1.69 (s, 4H); 2.07 (s, 3H); 2.64 (s, 6H); 6.93 (d, J=8.4 Hz, 1H); 7.13 (s, 1H); 7.15 (s, 1H); 7.58 (d, J=2.4 Hz, 1H); 7.75 (dd, J₁=8.7 Hz, J₂=2.1 Hz, 1H); 9.80 (s, 1H).

### Example 19

3-(3,5,5,8,8-Pentamethyl-5,6,7,8-tetrahydro-2-naphthyl)-4-chlorobenzaldehyde oxime;

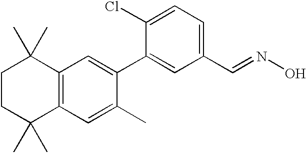

may be prepared in a similar manner as described in Example 1 using 4-chloro-3-(3,5,5,8,8-pentamethyl-5,6,7,8-tetrahydronaphtalen-2-yl)benzaldehyde.

The intermediate 4-chloro-3-(3,5,5,8,8-pentamethyl-5,6,7,8-tetrahydronaphtalen-2-yl)benzaldehyde was prepared as follows:

a. Ethyl 3-bromo-4-chlorobenzoate.

To a solution of 3-bromo-4-chlorobenzoic acid (3.00 g, 12.74 mmol) and cesium carbonate (6.23 g, 19.11 mmol) in acetonitrile (70 mL) was added iodoethane (5.1 mL, 63.7 mmol). The reaction mixture was heated at reflux overnight. After cooling to room temperature, the solution was extracted with ethyl acetate. The organic layer was washed successively with water and brine, dried over anhydrous magnesium sulfate, filtered and evaporated. Choromatography on silica gel (biotage, 5% EtOAc in hexane) afforded 3.5 g of ethyl 3-bromo-4-chlorobenzoate (97%). ¹H NMR (300 MHz; CDCl₃) δ1.40 (t, 3H); 4.37 (q, 2H); 7.52 (d, J=8.1 Hz, 1H); 7.91 (dd, J₁=8.4 Hz, J₂=1.8 Hz, 1H); 8.28 (d, J=1.8 Hz, 1H).

b. 3-Bromo-4-chloro-benzyl alcohol.

To a solution of ethyl-3-bromo-4-chlorobenzoate (3.25 g, 12.34 mmol) in toluene (70 mL) was added, at −78° C. under argon, diisobutylaluminum hydride (1.5M in toluene, 24 mL, 37.01 mmol). The reaction mixture was stirred at −78° C. for 1 hr then methanol (9 mL) and water (18 mL) was added. The solution was warmed up to room temperature and extracted with ethyl acetate. The organic layer was washed successively with water and brine, dried over anhydrous magnesium sulfate, filtered and evaporated to give 2.73 g of 3-bromo-4-chloro-benzyl alcohol.

c. 3-Bromo-4-chloro-benzaldehyde.

To a solution of 3-bromo-4-chlorobenzyl alcohol (2.73 g, 12.34 mmol) in dichloromethane (75 mL) was added, at room temperature, pyridinium chlorochromate (2.66 g, 12.34 mmol). The reaction mixture was stirred at room temperature for 1 hr then filtered over celite. The solvent was removed under reduced pressure and the residue chromatographed on silica gel (10% ethyl acetate in hexane) to afford 2.52 g of 3-bromo-4-chloro-benzaldehyde (93% yield). ¹H NMR (300 MHz; CDCl₃) ¹H NMR (300 MHz; CDCl₃) δ7.65 (d, J=8.1 Hz, 1H); 7.78 (dd, J₁=8.4 Hz, J₂=2.1 Hz, 1H); 8.12 (d, J=2.1 Hz, 1H).

d. 4-chloro-3-(3,5,5,8,7-pentamethyl-5,6,7,8-tetrahydronaphtalen-2-yl) benzaldehyde.

To a solution of 3-bromo-4-chlorobenzaldehyde (2.5 g, 11.39 mmol), (3,5,5,8,8-pentamethyl-5,6,7,8-tetrahydronaphtalen-2-yl) boronic acid (3.1 g, 12.53 mmol) in a mixture of toluene (25 mL), ethanol (5 mL) and water (4 mL) was added potassium carbonate (3.15 g, 22.78 mmol). The solution was degased with argon for 30 minutes. Tetrakis(triphenylphosphine)palladium(0) (0.26 g, 0.23 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was chromatographed on silica gel (biotage: eluant: ethyl acetate/hexane, 5:95) to give 3.0 g of 4-chloro-3-(3,5,5,8,7-pentamethyl-5,6,7,8-tetrahydronaphtalen-2-yl) benzaldehyde (77%). ¹H NMR (300 MHz; DMSO) δ1.18 (s, 3H); 1.20 (s, 3H); 1.24 (s, 3H); 1.26 (s, 3H); 1.36 (s, 4H); 1.98 (s, 3H); 7.04 (s, 1H); 7.23 (s, 1H); 7.75 (d, J=7.8 Hz, 1H); 7.80 (d, J=1.8 Hz, 1H); 7.88 (dd, J₁=7.8 Hz, J₂=1.8 Hz, 1H); 9.99 (s, 1H).

### Example 20

3-(3,5,5,8,8-Pentamethyl-5,6,7,8-tetrahydro-2-naphthyl)-4-trifluoromethoxybenzaldehyde oxime;

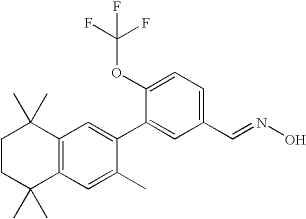

may be prepared in a similar manner as described in Example 1 using 4-trifluoromethoxy-3-(3,5,5,8,7-pentamethyl-5,6,7,8-tetrahydronaphthalen-2-yl) benzaldehyde.

The intermediate 4-trifluoromethoxy-3-(3,5,5,8,7-pentamethyl-5,6,7,8-tetrahydronaphthalen-2-yl) benzaldehyde was prepared as follows:

a. 3-Bromo-4-trifluoromethoxybenzaldehyde.

To a solution of 4-trifluoromethoxybenzaldehyde (215 g, 1.13 mol) in a mixture of TFA (300 mL), CH₂Cl₂ (300 mL) and H₂SO₄ (150 mL) was added at room temperature N-bromosuccinimide (402 g, 2.26 mol) in equal portion over 7 hours. The reaction mixture was stirred for 4 days at room temperature, poured into ice-water and extracted with CH₂Cl₂ The organic layer was washed with water then treated with saturated NaHCO₃ (1.5L) for 2 hrs. The layers were separated and the organic layer further washed with water and brine, dried over MgSO₄, filtered and evaporated. The residue was triturated with hexane and filtered. After evaporation of the solvent, the residue was distilled to give 3-bromo-4-trifluoromethoxybenzaldehyde (190.2 g, 81° C., 1.0 mm/Hg, 62%).

b. 4-Trifluoromethoxy-3-(3,5,5,8,7-pentamethyl-5,6,7,8-tetrahydronaphthalen-2-yl) benzaldehyde.

To a solution of 3-bromo-4-trifluoromethoxybenzaldehyde (10.0 g, 37.2 mmol), (3,5,5,8,8-pentamethyl-5,6,7,8-tetrahydronaphthalen-2-yl) boronic acid (11 g, 44.68 mmol, 1.2 eq) in a mixture of toluene (100 mL), ethanol (20 mL) and water (15 mL) was added potassium carbonate (10.28 g, 74.4 mmol, 2 eq). The solution was degassed with argon for 40 minutes. Tetrakis(triphenylphosphine)palladium(0) (0.86 g, 0.74 mmol, 0.02 eq) was added and the mixture heated at reflux under argon for 22 hours. The solution was cooled to room temperature, diluted with ethyl acetate and washed successively with water and brine, dried over MgSO₄, filtered and evaporated. The residue was chromatographed on silica gel (silica: 70-230 mesh, 60A, 400 g, eluant: ethyl acetate/hexane, 5:95) to give 4-trifluoromethoxy-3-(3,5,5,8,7-pentamethyl-5,6,7,8-tetrahydronaphthalen-2-yl) benzaldehyde (11.1 g, 76%). ¹H NMR (300 MHz; CDCl₃) δ1.25 (s, 6H); 1.32 (s, 6H); 1.70 (s, 4H); 2.08 (s, 3H); 7.06 (s, 1H); 7.18 (s, 1H); 7.48 (dd, J₁=8.4 Hz, J₂=1.5 Hz, 1H); 7.84 (d, J=2.0 Hz, 1H); 7.88 (dd, J₁=2.0 Hz , J₂=8.5 Hz 1H), 9.91 (s, 1H).

### Example 21

4-[3-(3,5,5,8,8-Pentamethyl-5,6,7,8-tetrahydro-2-naphthyl)-4-trifluoromethoxy-benzyloxy]benzaldehyde oxime;

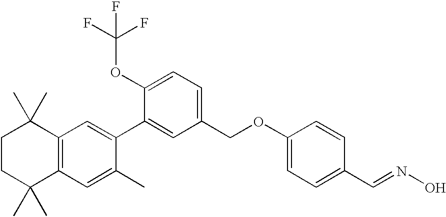

may be prepared in a similar manner as described in Example 1 using 4-[3-(3,5,5,8,8-pentamethyl-5,6,7,8-tetrahydro-2-naphthyl)-4-trifluoromethoxy-benzyloxy]benzaldehyde.

The intermediate 4-[3-(3,5,5,8,8-pentamethyl-5,6,7,8-tetrahydro-2-naphthyl)-4-trifluoromethoxy-benzyloxy]benzaldehyde was prepared as follows:

a. 3-(3,5,5,8,8-Pentamethyl-5,6,7,8-tetrahydro-2-naphthyl)-4-trifluoromethoxy benzyl alcohol.

To a solution of 4-trifluoromethoxy-3-(3,5,5,8,7-pentamethyl-5,6,7,8-tetrahydronaphthalen-2-yl) benzaldehyde (1.76 g, 4.51 mmol, see Example 20) in methanol at 0° C. was added NaBH₄ (0.170 g, 4.51 mmol) portionwise. After 30 minutes, the reaction was quenched with 10% acetic acid and the resulting mixture was extracted with ethyl acetate. The organics were washed sequentially with NaHCO₃, water and brine, dried over MgSO₄ and filtered. After evaporation, the residue was purified on silica gel (9:1 to 7:3, hexane:ethyl acetate) to afford 0.400 g (22%) of 3-(3,5,5,8,8-pentamethyl-5,6,7,8-tetrahydro-2-naphthyl)-4-trifluoromethoxy benzyl alcohol. ¹H NMR (500 MHz; CDCl₃) δ[1.25 (s), 1.31 (s), 12 H], 1.70 (s, 4H); 2.08 (s, 3H); 4.73 (s, 2 H), 7.07 (s, 1 H), 7.15 (s, 1H), 7.30-7.40 (m, 2 H), 7.38 (dd, J₁=2.0 Hz, J₂=8.9 Hz 1H).

b. 4-[3-(3,5,5,8,8-pentamethyl-5,6,7,8-tetrahydro-2-naphthyl)-4-trifluoromethoxy-benzyloxy]benzaldehyde.

To a solution of 3-(3,5,5,8,8-pentamethyl-5,6,7,8-tetrahydro-2-naphthyl)-4-trifluoromethoxy benzyl alcohol (0.400 g, 1 mmol) in DMF was added NaH (0.30 mg, 80% in mineral oil). After the evolution of hydrogen gas had stopped then 4-fluoro benzaldehyde (0.128 mL, 1.2 eq) was added and the mixture heat at 80° C. for 4 hours. The solution was cooled to room temperature and diluted with ethyl acetate and washed successively with water and brine, dried over MgSO₄, filtered and evaporated. The residue was purified on silica gel (hexane to 7:3 hexane:ethyl acetate) to afford 0.80 g (16%) of 4-[3-(3,5,5,8,8-pentamethyl-5,6,7,8-tetrahydro-2-naphthyl)-4-trifluoromethoxy-benzyloxy]benzaldehyde. ¹H NMR (500 MHz; CDCl₃) δ[1.25 (s), 1.31 (s), 12 H], 1.70 (s, 4H); 2.06 (s, 3H); 5.17 (s, 2 H), 7.08 (d, J₁=9.0 Hz, 2 H), 7.08 (s, 1 H), 7.16 (s, 1H), 7.30-7.40 (m, 2 H), 7.44 (dd, J₁=3.0 Hz , J₂=8.5 Hz 1H), 9.90 (s, 1 H).

### Example 22

4-[2-(Methyl-pyridin-2-yl-amino)-ethoxy] benzaldehyde oxime;

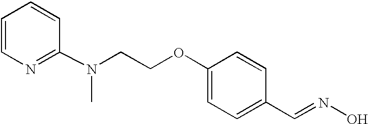

may be prepared in a similar manner as described in Example 1 using 4-[2-(methylpyridin-2-yl-amino)-ethoxy] benzaldehyde.

The intermediate 4-[2-(methyl-pyridin-2-yl-amino)-ethoxy] benzaldehyde was prepared as described by Cantello, et al., Bioorganic & Medicinal Chemistry Letters, 1994, 4, 1181-1184).

### Example 23

2-(3,5,5,8,8-Pentamethyl-5,6,7,8-tetrahydro-2-naphthyl)-3-methoxybenzaldehyde oxime;

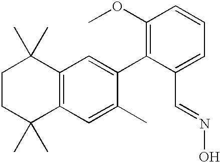

may be prepared in a similar manner as described in Example 1 using 3-methoxy-2-(3,5,5,8,8-pentamethyl-5,6,7,8-tetrahydronaphthalen-2-yl)benzaldehyde.

The intermediate 3-methoxy-2-(3,5,5,8,8-pentamethyl-5,6,7,8-tetrahydronaphthalen-2-yl)benzaldehyde was prepared as follows:

a. To a solution of o-vanillin (0.5 g, 3.28 mmol; i.e., 3-methoxy-2-hydroxybenzaldehyde) in dichloromethane (20 mL) was added pyridine (0.3 mL, 1.2 eq) and the solution cooled to 0° C. Triflic anhydride (0.65 mL, 1.2 eq) was added slowly and the resulting reaction mixture was allowed to warm slowly to room temperature and stirred for 3 hr. at room temperature. The solution was washed successively with water and brine, dried over anhydrous magnesium sulfate, filtered and evaporated. The residue was purified on silica gel (ethyl acetate/hexane, 1:9) to give 0.437 g of 3-methoxy-2-trifluoromethanesulfonyl benzaldehyde (yield 47%). The product was used without further purification.

b. A mixture of 3-methoxy-2-trifluoromethanesulfonyl benzaldehyde (0.430 g, 1.51 mmol), (3,5,5,8,8-pentamethyl-5,6,7,8-tetrahydronaphtalen-2-yl) boronic acid (0.740 g, 3.00 mmol) and potassium carbonate (0.835 g) in 1,2-dimethoxyethane (20 mL) and water (1 mL) was degassed with argon for 15 minutes. To this mixture was added tetrakis(triphenylphosphine)palladium(0) (0.35 g, 0.3 mmol) and the resulting mixture was heated at reflux under argon for 4 hours. The solution was cooled to room temperature, diluted with ethyl acetate and washed successively with water and brine, dried over anhydrous magnesium sulfate, filtered and evaporated. The residue was chromatographed on silica gel (ethyl acetate/hexane, 1:9) to give 0.48 g of 3-methoxy-2-(3,5,5,8,8-pentamethyl-5,6,7,8-tetrahydronaphthalen-2-yl)benzaldehyde.

### Example 24

### Oral Administration of Compound 1 in the Treatment of Hypercholesterolemia in Sprague Dawley Rats Maintained on a High Cholesterol Atherogenic Diet

### METHODS

Animals and Housing

Six week-old male Sprague Dawley rats (HSD, Harlan) were housed in a fixed 12-12- hr artificial light-dark cycle, and maintained on a high cholesterol, atherogenic diet ad libitum (#C13002, Research Diets, NJ).

Animals were maintained on this diet throughout the course of the study.

Dosage Groups and Treatment

Following six days of maintenance on the high cholesterol diet, the animals were bled from the tail vein (100-200 μL of whole blood) and serum levels of total cholesterol were measured in duplicate (Infinity Cholesterol Kit; Sigma, St. Louis, Mo.). Based on these initial measures, animals were sorted into groups with approximately the same average serum cholesterol levels. Once sorted, the animals were housed three per cage and maintained on the high cholesterol diet ad libitum.

Experiment I: (Compound 1)

Treatment groups(n=6/group):

1) Lean Sprague Dawley control (sesame oil)

2) High cholesterol fed control (sesame oil)

3) Compound 1 in sesame oil (10 mg/kg; once daily)

Drug is prepared by mixing Compound 1 in sesame oil, and administered to animals in a volume of 3 ml/kg/dose. Drug is administered by oral gavage daily for five consecutive days.

Serum Measurements

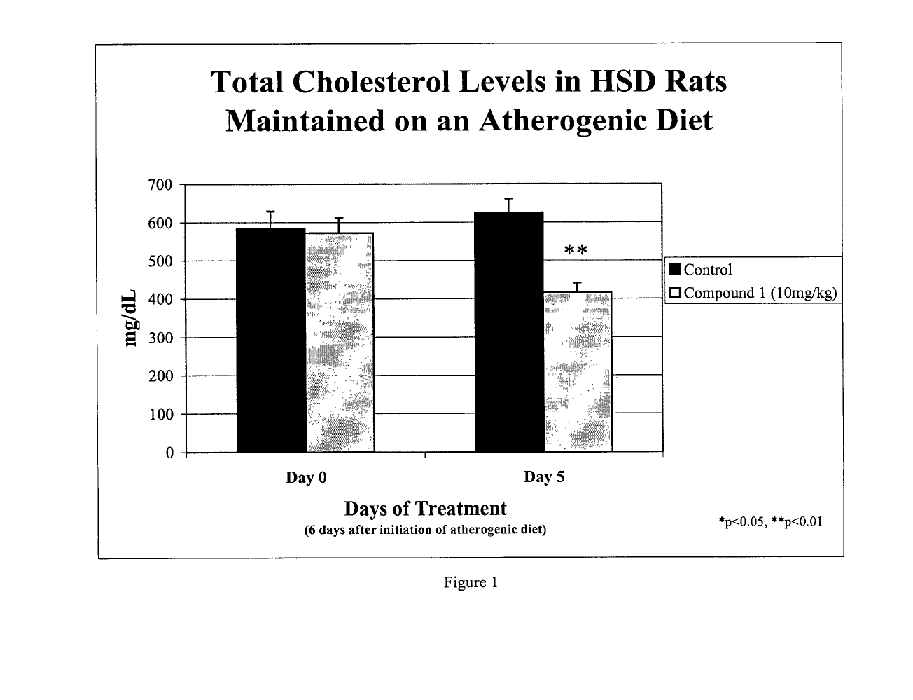

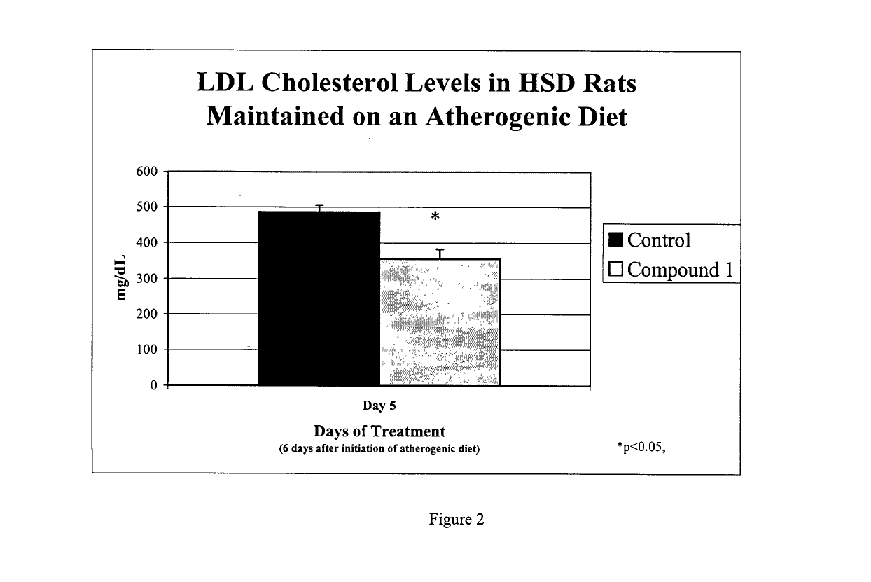

To monitor the effect of Compound 1, animals were bled from the tail vein five days after commencement of oral treatment. Serum cholesterols were measured in duplicate. The blood is kept at room temperature to allow coagulation, after which the serum is separated and assayed for total cholesterol and low density lipoprotein cholesterol levels. As shown in FIG. 1, Compound 1 significantly reduced total serum cholesterol compared to control animals maintained on the same atherogenic diet (ANOVA, Fisher's Least significant difference test, p≦0.01). Similarly, compound 1 reduced LDL cholesterol levels (FIG. 2) compared to controls (ANOVA, Fisher's Least significant difference test, p≦0.0 1).

### Example 25

### Oral Administration of Compound 1 in the Treatment of Obese, Glucose Intolerant Zucker Fatty Rats (fa/fa)

### METHODS

Animals and Housing

Ten week-old male Zucker Fatty Rats (fa/fa; Harlan) were housed in a fixed 12-12- hr artificial light-dark cycle, and maintained on a standard diet provided ad libitum. Animals were allowed two days to acclimate in this experimental environment prior to the initiation of the study.

Dosage Groups and Treatment

Prior to initiation of treatment, the animals were bled from the tail vein (100-200 μL of whole blood) and serum levels of triglycerides were measured in duplicate (Infinity kits; Sigma, St. Louis, Mo.). Based on these initial measures, animals were sorted into groups with approximately the same average serum triglyceride levels. Once sorted, the animals were housed one per cage and provided standard rodent diet ad libitum.

Treatment groups (n=5/group):

1) Control (sesame oil)

2) Compound 1 in sesame oil (10 mg/kg once daily)

Drug is prepared by mixing Compound 1 in sesame oil, and administered to animals in a volume of 3 ml/kg/dose. Drug is administered by oral gavage once daily.

Serum Measurements

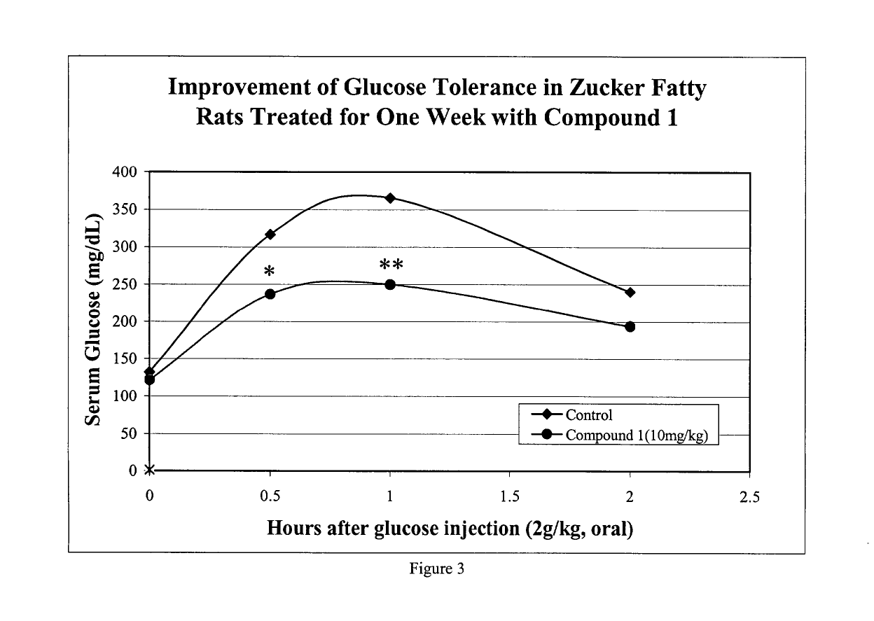

To monitor the effect of Compound 1, animals were tested in a glucose tolerance test. One day prior to the tolerance test, the animals were fasted overnight (12 hr fast). Animals were then bled at 0 hours to establish baseline glucose levels. Following this initial bleed the animals were given 2 g/kg glucose administered in a 40% glucose solution by oral gavage. The animals were then bled 0.5, 1, and 2 hours later and serum glucose levels were measured in duplicate. The blood is kept at room temperature to allow coagulation, after which the serum is separated and assayed for glucose levels. As shown in FIG. 3, Compound 1 produced a significant increase glucose clearance rate following 1 week of treatment (*p<0.05 and **p<0.01; ANOVA and Fisher's Least Significant Difference; FIG. 3).

### Example 26

3-(5-Isobutyl-3,3-dimethyl-2,3-dihydro-benzofuran-7-yl)-4-trifluoromethoxy-benzaldehyde oxime

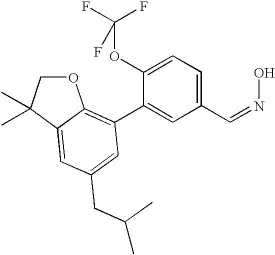

A mixture of 3-(5-isobutyl-3,3-dimethyl-2,3-dihydro-benzofuran-7-yl)-4-trifluoromethoxy-benzaldehyde and hydroxylamine sulfate is reacted in a basic solution to form the illustrated compound, as a mixture of syn and anti isomers.

The intermediate 3-(5-isobutyl-3,3-dimethyl-2,3-dihydro-benzofuran-7-yl)-4-trifluoromethoxy-benzaldehyde was prepared as follows:

a. 3-(5-isobutyl-3,3-dimethyl-2,3-dihydro-benzofuran-7-yl)-4-trifluoromethoxy-benzaldehyde.

A mixture of 3-bromo-4-trifluoromethoxy benzaldehyde (4.24 g, 15.75 mmol), 5-isobutyl-3,3-dimethyl-2,3-dihydro-benzofuran-7-boronic acid (4.3 g, 17.33 mmol) and potassium carbonate (4.35 g, 31.5 mmol) in toluene (39 mL), ethanol (7.5 mL) and water (2.5 mL) was degassed with argon for 15 minutes.

Tetrakis(triphenylphosphine)palladium(0) (0.728 g, 0.63 mmol) was added and the mixture heated at reflux under argon for 20 hrs. The solution was cooled to room temperature, diluted with ethyl acetate and washed successively with water and brine, dried over anhydrous magnesium sulfate, filtered and evaporated. The residue was purified on silica gel (0 to 5% ethyl acetate in hexane) to give 5.76 g of 3-(5-isobutyl-3,3-dimethyl-2,3-dihydro-benzofuran-7-yl)-4-trifluoromethoxy-benzaldehyde (93%). ¹H NMR (300 MHz; CDCl₃): 0.92 (d, J=6.9 Hz, 6 H), 1.36 (s, 6 H), 1.84 (m, 1 H), 2.47 (d, J=7.5 Hz, 2 H), 4.22 (s, 2 H), 6.92 (d, J=4.8 Hz, 2 H), 7.46 (dd, J=1.5 Hz and 8.7 Hz, 1 H), 7.90 (dd, J=2.1 Hz and 8.7 Hz, 1 H), 8.03 (d, J=2.1 Hz, 1 H), 10.03 (s, 1 H).

b. 5-isobutyl-3,3-dimethyl-2,3-dihydro-benzofuran-7-boronic acid.

To a mixture of 7-bromo-5-isobutyl-3,3-dimethyl-2,3-dihydro-benzofuran (9.9 g, 34.96 mmol) in THF (50 mL) cooled to −78° C. under an atmosphere of argon was added n-BuLi (25.17 mL, 2.5M, 62.93 mmol) dropwise. The reaction mixture was stirred for 5 minutes and triisopropylborate (24.2 mL, 104.87 mmol) was added dropwise. The mixture was stirred at −50° C. for 2 hours then warmed up to room temperature and stirred overnight at room temperature. 1.0N HCl (100 mL) was slowly added to the reaction mixture. After 1 hour the mixture was diluted with ethyl acetate and the layers separated. The organic layer was further washed with water, brine, dried (Mg₂SO₄), filtered and evaporated. The residue was chromatographed on silica gel (0 to 20% ethyl acetate in hexane) to give 4.3 g of 5-isobutyl-3,3-dimethyl-2,3-dihydrobenzofuran-7-boronic acid (46%). ¹H NMR (300 MHz; CDCl₃): 0.90 (d, J=6.6 Hz, 6 H), 1.33 (s, 6 H), 1.81 (m, 1 H), 2.43 (d, J=7.5 Hz, 2 H), 4.28 (s, 2 H), 5.86 (br s, 2 H), 6.98 (d, J=2.1 Hz, 1 H), 7.33 (d, J=2.1 Hz, 1 H).

c. 7-bromo-5-isobutyl-3,3-dimethyl-2,3-dihydro-benzofuran.

To a solution of 5-isobutyl-3,3-dimethyl-2,3-dihydro-benzofuran (1.59 g, 7.78 mmol) in dichloromethane (40 mL) was added pyridinium tribromide (2.49 g, 7.78 mmol) and the reaction mixture stirred at room temperature overnight. The solution was washed with water and brine, dried (Mg₂SO₄), filtered and evaporated. The residue was purified on silica gel (0% to 2% ethyl acetate in hexane) to give 1.51 g of 7-bromo-5-isobutyl-3,3-dimethyl-2,3-dihydro-benzofuran (68%). ¹H NMR (300 MHz; CDCl₃): δ0.90 (d, J=6.3 Hz, 6 H), 1.33 (s, 6 H), 1.77 (m, 1 H), 2.39 (d, J=7.5 Hz, 2 H), 4.30 (s, 2 H), 6.80 (d, J=1.5 Hz, 1 H), 7.05 (d, J=1.5 Hz, 1 H).

d. 5-isobutyl-3,3-dimethyl-2,3-dihydro-benzofuran.

To a cold solution (0° C.) of 1-(3,3-dimethyl-2,3-dihydro-benzofuran-5-yl)-2-methyl-propan-1-ol (1.97 g, 8.93 mmol) in dry dichloromethane (40 mL) was added triethylsilane (2.85 mL, 17.86 mmol). After 10 minutes, trifluoroacetic acid was the reaction mixture stirred at 0° C. for 30 minutes. Water was poured into the reaction mixture and the layers separated. The organic layer was further washed with water, aqueous NaHCO₃ and brine, dried (Mg₂SO₄), filtered and evaporated. The residue was purified on silica gel (0% to 5% ethyl acetate in hexane) to give 1.6 g of 5-isobutyl-3,3-dimethyl-2,3-dihydro-benzofuran (87%). ¹H NMR (300 MHz; CDCl₃): δ0.90 (d, J=6.3 Hz, 6 H), 1.32 (s, 6 H), 1.79 (m, 1 H), 2.40 (d, J=6.9 Hz, 2 H), 4.20 (s, 2 H), 6.68 (dd, J=1.2 Hz and 7.5 Hz, 1 H), 6.87 (m, 2 H).

e. 1-(3,3-dimethyl-2,3-dihydro-benzofuran-5-yl)-2-methyl-propan-1-ol.

To a solution of 5-bromo-3,3-dimethyl-2,3-dihydro-benzofuran (2.03 g, 8.93 mmol) in dry THF (10 mL) at −78° C., under argon, was added dropwise n-BuLi (1.6M in hexane, 13.4 mmol, 8.38 mL). The mixture was stirred for 5 minutes then isobutyraldehyde (1.22 mL, 8.38 mmol) was added and the mixture was slowly warmed up to room temperature and stirred overnight at room temperature. Aqueous ammonium chloride was added and the solution extracted with ethylacetate and the organic extract was dried (Mg₂SO₄), filtered and evaporated. The residue was purified on silica gel (0% to 20% ethyl acetate in hexane) to give 1.97 g of 1-(3,3-dimethyl-2,3-dihydrobenzofuran-5-yl)-2-methyl-propan-1-ol (100%). ¹H NMR (300 MHz; CDCl₃): δ0.77 (d, J=6.6 Hz, 3 H), 0.90 (d, J=6.6 Hz, 3 H), 1.33 (s, 6 H), 1.95 (m, 1 H), 4.23 (s, 2 H), 4.28 (d, J=7.2 Hz, 2 H), 6.72 (d, J=8.4 Hz, 1 H), 7.03 (dd, J=8.1 Hz and 1.8 Hz, 1 H), 7.06 (d, J=1.5 Hz, 1 H).

f. 5-bromo-3,3-dimethyl-2,3-dihydro-benzofuran

A mixture of 4-bromo-2-(2-chloro-1,1-dimethyl-ethyl)-1-methoxy-benzene (65 g, 0.234 mol), pyridine hydrochloride (121.8 g, 1.054 mol) and quinoline (110.67 mL, 0.936 mol) was refluxed at 164° C.-167° C. under argon for 5 hrs. After cooling to room temperature the reaction mixture was treated with ice-cold 6N HCl and extracted twice with ether. The organic layers were combined, dried (Mg₂SO₄), filtered and evaporated. The residue was purified on silica gel (10% ethyl acetate in hexane) to give 52 g of 5-bromo-3,3-dimethyl-2,3-dihydro-benzofuran (98%). ¹H NMR (300 MHz; CDCl₃): δ1.32 (s, 61H), 4.23 (s, 2 H), 6.67 (d,J=8.1 Hz, 1 H), 7.19 (m, 2 H).

g. 4-bromo-2-(2-chloro-1,1-dimethyl-ethyl)-1-methoxy-benzene.

Sulfuric acid (2 mL, 0.033 mol) was added dropwise under argon to 4-bromoanisole (14.6 mL, 0.117 mol). The mixture was warmed to 40-43° C. (warm water bath) and 3-chloro-2-methyl propene was added dropwise in 4 equal portions over 2 hrs. After 2 hrs at 40-43° C. the solution was diluted with dichloromethane and washed successively with water, saturated aqueous NaHCO₃, water and brine, dried (Mg₂SO₄), filtered and evaporated. The residue was crystallized from hexanes to give 14.1 g of 4-bromo-2-(2-chloro-1,1-dimethyl-ethyl)-1-methoxy-benzene. The mother liquor was further purified on silica gel (10% ethyl acetate in hexane) to afford additional 4.8 g of product. 58% yield. ¹H NMR (300 MHz; CDCl₃): δ1.43 (s, 6 H), 3.82 (s, 3 H), 3.93 (s, 2 H), 6.75 (dd, J=2.4 Hz and 7.2 Hz, 1 H), 7.32 (m, 2 H).

### Example 27

3-(1,4,4,6-Tetramethyl-2-oxo-1,2,3,4-tetrahydro-quinolin-7-yl)-4-trifluoromethoxy-benzaldehyde oxime.

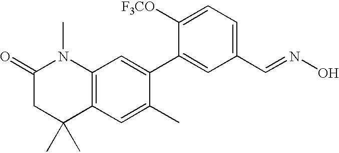

A mixture of 3-(1,4,4,6-Tetramethyl-2-oxo-1,2,3,4-tetrahydro-quinolin-7-yl)-4-trifluoromethoxy-benzaldehyde and hydroxylamine sulfate is reacted in a basic solution to form the illustrated oxime compound.

The intermediate 3-(1,4,4,6-tetramethyl-2-oxo-1,2,3,4-tetrahydro-quinolin-7-yl)-4-trifluoromethoxy-benzaldehyde was prepared as follows:

a. 3-(1,4,4,6-Tetramethyl-2-oxo-1,2,3,4-tetrahydro-quinolin-7-yl)-4-trifluoromethoxy-benzaldehyde.

A mixture of 3-formyl-6-trifluoromethoxy-1-phenyl boronic acid (3.14 g, 13.42 mmol), 7-bromo-1,4,4,6-tetramethyl-3,4-dihydro-1H-quinoline-2-one (3.15 g, 11.19 mmol) and potassium carbonate (3.1 g, 22.38 mmol) in toluene (35 mL), ethanol (11.8 mL) and water (7.3 mL) was degassed with argon for 15 minutes. Tetrakis(triphenylphosphine)palladium(0) (0.259 g, 0.02 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was purified on silica gel (20 to 30% ethyl acetate in hexane) to give 2.34 g of 3-(1,4,4,6-tetramethyl-2-oxo-1,2,3,4-tetrahydro-quinolin-7-yl)-4-trifluoromethoxy-benzaldehyde (54%). ¹H NMR (300 MHz; CDCl₃): 1.35 (s, 6 H), 2.11 (s, 3 H), 2.55 (s, 2 H), 3.35 (s, 3 H), 6.79 (s, 1 H), 7.20 (s, 1 H), 7.54 (dd, J=3 and 8.4 Hz, 1 H), 7.85 (d, J=2.7 Hz, 1 H), 7.90 (dd, J=2.1 and 8.7 Hz, 1 H), 10.04 (s, 1 H).

b. 3-formyl-6-trifluoromethoxy-1-phenyl boronic acid.

To a mixture of 2-(3-bromo-4-trifluoromethoxy-1-phenyl)-1,3-dioxolane (7.20 g, 22.9 mmol) in THF (70 mL) cooled to −78° C. under an atmosphere of argon was added n-BuLi (13.8 mL, 2.5M, 34.4 mmol) dropwise. The resulting suspension was stirred for 5 minutes and triisopropylborate (15.9 mL, 68.7 mmol) was added dropwise via syringe. The mixture was stirred at −50° C. for 2 hours then warmed up to room temperature and stirred overnight at room temperature. 1.0N HCl (50 mL) was slowly added to the reaction mixture. After 3 hours the mixture was diluted with ethyl acetate and the layers separated, the aqueous layer was extracted once with ethyl acetate and the two organic layers combined. The resulting organic layer was washed with water, brine and dried (Mg₂SO₄). The mixture was filtered, evaporated and the residue stirred in hexane. The resulting white suspension was filtered and the white solid dried under high vacuum to afford 3.00 g of 3-formyl-6-trifluoromethoxy-1-phenyl boronic acid (56%). ¹H NMR (300 MHz; CDCl₃): δ7.42 (d, J=7.0 Hz, 1 H), 8.07 (dd, J₁=2.1 Hz, J₂=8.7 Hz, 1 H), 8.47 (d, J=1.8 Hz, 1 H), 10.05 (s, 1 H).

c. 2-(3-bromo-4-trifluoromethoxy-1-phenyl)-1,3-dioxolane.

To a solution of 3-bromo-4-trifluoromethoxybenzaldehyde (20 g, 74.0 mmol) in toluene (200 mL) was added ethylene glycol (82.6 mL, 1.48 mol) and p-toluenesulfonic acid monohydrate (0.84 g, 4.44 mmol). The reaction mixture was heated at reflux overnight and the water was removed using a Dean Stark apparatus. The solution was cooled to room temperature, poured into aqueous potassium carbonate (10%) and extracted with ethyl acetate. The organic layer was washed with water, brine and dried (Mg₂SO₄). The residue was purified on silica gel (eluent: 10% ethyl acetate in hexane) to give 15.4 g of 2-(3-bromo-4-trifluoromethoxy)-1,3-dioxolane (66%). ¹H NMR (500 MHz; CDCl₃): δ4.05 (m, 2 H), 4.11 (m, 2 H), 5.79 (s, 1 H), 7.32 (d, 1 H), 7.43 (d, 1 H),7.77 (d,J=1.1 Hz, 1 H).

d. 7-bromo-1,4,4,6-tetramethyl-3,4-dihydro-1H-quinoline-2-one.

A mixture of powdered KOH (14.06 g, 0.250 mol) in DMSO (150 mL) was stirred at 0° C. for 10 min. 7-Bromo-4,4,6-trimethyl-3,4-dihydro-1H-quinoline-2-one (33.59 g, 0.125 mol) was added cautiously, followed immediately by the addition of methyl iodide (39 mL, 0.625 mol). The reaction mixture was kept at 0° C. for 30 min then slowly warmed up to room temperature and stirred overnight at room temperature. The reaction mixture was poured into water and extracted with dichloromethane washed with water and brine, dried (Mg₂SO₄), filtered and evaporated to give 35.74 g of 7-bromo-1,4,4,6-tetramethyl-3,4-dihydro-1H-quinoline-2-one (99%) and used without further purification in the Suzuki coupling (step a). ¹H NMR (300 MHz; CDCl₃): 1.27 (s, 6 H), 2.37 (s, 3 H), 2.48 (s, 2 H), 3.35 (s, 3 H), 7.12 (s, 1 H), 7.16 (s, 1 H).

e. 7-bromo-4,4,6-trimethyl-3,4-dihydro-1H-quinoline-2-one.

To a solution of 3-methyl-but-2-enoic acid (3-bromo-4-methyl-phenyl)-amide (70.0 g, 261 mmol) at 90° C. was added portion wise, under argon, with vigorous stirring aluminum chloride (52.3 g, 391 mmol) over 1.5 hr. The reaction mixture was stirred for 2 hours at 110-120° C. The reaction mixture was cooled to room temperature and ice-water was carefully added. The solution was extracted with dichloromethane and the organic washed with 2N HCl, water, saturated aqueous NaHCO₃, water and brine, dried (Mg₂SO₄), filtered and evaporated. The residue was crystallized from dichloromethane/hexane to give 46 g of 7-bromo-4,4,6-trimethyl-3,4- dihydro-1H-quinoline-2-one. The mother liquor was further chromatographed on silica gel(20% ethyl acetate in hexane) to give 6.2 g more of product. (75%). ¹H NMR (300 MHz; CDCl₃): 1.30 (s, 6 H), 2.33 (s, 3 H), 2.46 (s, 2 H), 7.07 (s, 1 H), 7.10 (s, 1 H), 9.87 (br s, 1 H).

f. 3-Methyl-but-2-enoic acid (3-bromo-4-methyl-phenyl)-amide.

To a biphasic mixture of 3-bromo-4-methylaniline (50 g, 0.269 mol), 10% NaOH (270 mL) and dichloromethane (160 mL) was added dropwise over a period of 2 hours 3,3-dimethylacryloyl chloride (36 mL, 0.322 mol) in dichloromethane (95 mL). The solution was stirred at room temperature for 48 hours then diluted with water (100 mL). The aqueous layer was further extracted with dichloromethane. The organic layers were combined and washed with water and brine, dried (Mg₂SO₄), filtered and evaporated. The white solid was triturated with hexane and collected to give 70 g (97%) of 3-Methyl-but-2-enoic acid (3-bromo-4-methyl-phenyl)-amide. ¹H NMR (300 MHz; CDCl₃): 1.89 (s, 3 H), 2.21 (s, 3 H), 2.33 (s, 3 H), 5.68 (s, 1 H), 7.14 (d, J=8.0 Hz, 1 H), 7.17 (br s, 1 H), 7.33 (d, J=8.0 Hz, 1 H), 7.79 (s, 1 H).

g. 3-bromo-4-methylaniline.

To a solution of 2-bromo-4-nitrotoluene (50 g, 0.231 mol in ethylacetate (330 mL) and Ethanol (150 mL) was added Tin(II)chloride dihydrate (208 g, 0.924 mol) portionwise. The reaction mixture was stirred at room temperature overnight. The solution was then treated with potassium carbonate until pH=7 and filtered over celite. The filtrate was washed with water, aqueous NaHCO₃, water and brine, dried (Mg₂SO₄), filtered and evaporated to give 42.71 g (100%) of 3-bromo-4-methylaniline. ¹H NMR (300 MHz; CDCl₃): 2.27 (s, 3 H), 3.57 (br s, 2 H), 6.54 (dd, J=2.7 Hz and 8.1 Hz, 1 H), 6.90 (d, J=2.1 Hz, 1 H), 6.98 (d, J=8.1 Hz, 1 H).

### Example 28

4-Dimethylamino-3-(1,4,7-trimethyl-2,3-dioxo-1,2,3,4-tetrahydroquinoxalin-6-yl)-benzaldehyde oxime.

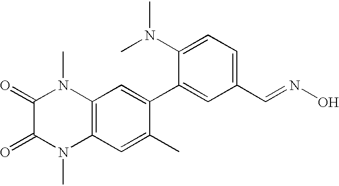

The compound is prepared by reacting 4-Dimethylamino-3-(1,4,7-trimethyl-2,3-dioxo-1,2,3,4-tetrahydro-quinoxalin-6-yl)-benzaldehyde and hydroxylamine sulfate in a basic solution.

The intermediate 4-Dimethylamino-3-(1,4,7-trimethyl-2,3-dioxo-1,2,3,4-tetrahydro-quinoxalin-6-yl)-benzaldehyde was prepared in a similar manner to example 3a using 6-dimethylamino-3-formyl-1-phenyl boronic acid (example 3b) and 6-bromo-1,4,7-trimethyl-1,4-dihydro-quinoxaline-2,3-dione (18%). ¹H NMR (300 MHz; CDCl₃): 2.12 (s, 3 H), 2.69 (s, 6 H), 3.65 (s, 6 H), 7.1-7.6 (m, 5 H), 9.84 (s, 1 H).

a. 6-bromo-1,4,7-trimethyl-1,4-dihydro-quinoxaline-2,3-dione.

To a solution of 1,4,6-trimethyl-1,4-dihydro-quinoxaline-2,3-dione (0.66 g, 3.2 mmol) in acetic acid (40 mL) was added bromine (0.52 g, 3.2 mmol) and the solution stirred at 50° C. overnight. The reaction mixture was cooled to room temperature and poured into water. The solution was neutralized with aqueous NaOH to Ph=7, extracted with dichloromethane and washed with brine, dried (Mg₂SO₄), filtered and evaporated to give 0.9 g of 6-bromo-1,4,7-trimethyl-1,4-dihydro-quinoxaline-2,3-dione used without further purification in the Suzuki coupling (step a). ¹H NMR (300 MHz; CDCl₃): 2.47 (s, 3 H), 3.64 (s, 6 H), 7.09 (s, 1 H), 7.40 (s, 1 H).

b. 1,4,6-trimethyl-1,4-dihydro-quinoxaline-2,3-dione.

To a solution of 6-methyl-1,4-dihydro-quinoxaline-2,3-dione (5.3 g, 30 mmol) in THF (150 mL) was added, at 0° C. under argon, sodium hydride (3.68 g, 80% in mineral oil, 120 mmol) followed by methyl iodide (7.5 mL, 120 mmol). The solution was stirred at 0° C. for 3 hrs and at room temperature overnight. The reaction mixture was cooled to 0° C. and acidified with IN HCl. The solution was extracted with dichloromethane washed with brine, dried (Mg2SO4), filtered and evaporated. The residue was chromatographed on silica gel (10 to 25% acetonitrile in dichloromethane) to give 1.1 g of 1,4,6-trimethyl-1,4-dihydro-quinoxaline-2,3-dione (18%). ¹H NMR (300 MHz; CDCl₃): 2.44 (s, 3 H), 3.66 (s, 6 H), 7.06-7.15 (m, 3 H).

c. 6-methyl-1,4-dihydro-quinoxaline-2,3-dione.

3,4-Diaminotoluene (24.4 g, 0.2 mmol) was dissolved in 2N HCl (300 mL), oxalic acide dihydrate (27.7 g, 0.22 mmol) was added and the mixture was heated at reflux for 3.5 hrs. The reaction mixture was cooled to room temperature, filtered, washed with water, dried (Mg₂SO₄), filtered and evaporated to give 34 g of 6-methyl-1,4-dihydro-quinoxaline-2,3-dione (96%). ¹H NMR (300 MHz; CDCl₃): 2.25 (s, 3 H), 6.87-6.99 (m, 3 H), 11.87 (br s, 2H).

### Example 29

4-(7-Adamantan-1-yl-benzo[1,3]dioxol-5-yl)-benzaldehyde oxime

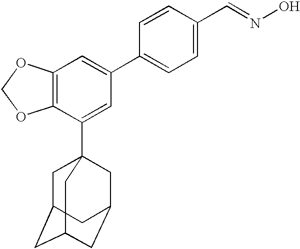

A solution of 4-[3-(1-adamantyl)-4,5-methylenedioxyphenyl]-benzaldehyde and hydroxylamine sulfate is reacted to produce the illustrated compound.

The intermediate 4-[3-(1-Adamantyl)-4,5-methylenedioxyphenyl]-benzaldehyde was prepared as follows:

a. 4-[3-(1-Adamantyl)-4,5-methylenedioxyphenyl]-benzaldehyde.

A mixture of 3-(1-adamantyl)-4,5-methylenedioxy-1-bromobenzene (2.00 g, 5.97 mmol), 4-formylphenylboronic acid (1.07 g, 7.16 mmol) and potassium carbonate (1.86 g, 13.42 mmol) in 1,2-dimethoxyethane (50 mL) and water (2.5 mL) was degassed with argon for 30 minutes. Tetrakis(triphenylphosphine)palladium(0) (0.34 g, 0.298 mmol) was added and the mixture heated at reflux under argon overnight. The solution was cooled to room temperature, diluted with ethyl acetate (200 mL) and washed successively with water (100 mL) and brine (100 mL), dried over anhydrous magnesium sulfate, filtered and evaporated. The residue was purified on silica gel (eluent:hexane:ethyl acetate, 95:5) to give 1.82 g of 4-[3-(1-Adamantyl)-4,5-methylenedioxy phenyl]-benzaldehyde (85%). ¹H NMR (500 MHz; CDCl₃): δ1.79 (s, 6 H); 2.08 (s, 9 H); 6.01 (s, 2 H); 7.00 (d, J=2.0 Hz, 1 H); 7.04 (d, J=2.0 Hz, 1 H); 7.68 (d, J=8.1 Hz, 2 H); 7.91 (d,J=8.1 Hz, 2 H); 10.03 (s, 1 H).

b. 3-(1-Adamantyl)-4,5-methylenedioxy-1-bromobenzene.

To a mixture of 3,4-methylenedioxy-1-bromobenzene (5.00 g, 24.87 mmol) and 1-adamantanol (3.79 g, 24.87 mmol) in CH₂Cl₂ (50 mL) under an atmosphere of argon was added sulfuric acid (2.0 mL) at room temperature. After stirring for 3 days the resulting mixture was diluted with CH₂Cl₂ and washed with water. The aqueous layer was extracted with CH₂Cl₂ and the combined organics were washed successively with water, brine and dried (MgSO₄). The mixture was filter, evaporated and the residue purified on silica gel (hexane) to give 4.41 g of 3-(1-adamantyl)-4,5-methylenedioxy-1-bromobenzene (53%) as a white solid, mp 135.5-136.0° C.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1) Compounds of the formula 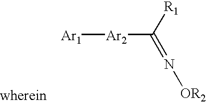
a) Ar₁ comprises a substituted aryl or heteroaryl ring wherein two substituents together with the aryl or heteroaryl ring of Ar₁ together form an additional cycloalkyl, substituted cycloalkyl, cycloalkenyl or substituted cycloalkenyl ring radicals optionally comprising 1 or 2 ring heteroatoms selected from O, S, SO, SO₂ and N, wherein N is further substituted with hydrogen, alkyl or substituted alkyl;
b) Ar₂ is a substituted or unsubstituted aryl radical or a substituted or unsubstituted heteroaryl radical;
c) R₁ is hydrogen, a substituted or unsubstituted amino radical, or a substituted or unsubstituted organic radical comprising from one to 12 carbon atoms; and
d) R₂ is hydrogen, or a substituted or unsubstituted organic radical comprising from one to 12 carbon atoms; or a pharmaceutically acceptable salt thereof.

2) The compound of claim 1, wherein the Ar₁ aryl or heteroaryl ring and the additional cyclic ring radical bonded thereto have 1, 2, 3, 4, 5, 6, or 7 non-hydrogen substituent groups, and Ar₁ and its substitutent groups together comprise between 6 and 30 carbon atoms.

3) The compound of claim 2, wherein the non-hydrogen substitutent groups are independently selected from the group consisting of an alkyl, substituted alkyl, alkenyl, substituted alkenyl, alkynyl, substituted alkynyl, halogen, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, or alkylsulfonamide radical.

4) The compound of claim 1, wherein the additional cycloalkyl, substituted cycloalkyl, cycloalkenyl or substituted cycloalkenyl ring radical bonded to the aryl or heteroaryl ring of Ar₁ comprises from 1 to 8 additional ring carbon atoms exocyclic to the aryl or heteroaryl ring.

5) The compound of claim 1, wherein Ar₁ has the formula: 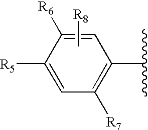
wherein: R₅ and R₆ together with the aromatic ring form a cycloalkyl, substituted cycloalkyl, cycloalkenyl or substituted cycloalkenyl optionally comprising 1 or 2 heteroatoms selected from O, S, SO, SO₂ and N, wherein N is further substituted with hydrogen, alkyl or substituted alkyl; and R₇ and R₈ are independently or together selected from the group consisting of hydrogen, alkyl, substituted alkyl, alkenyl, substituted alkenyl, alkynyl, substituted alkynyl, halogen, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, and alkylsulfonamide radical.

6) The compound of claim 1, wherein Ar₁ comprises a substituted or unsubstituted ring radical of the formula:

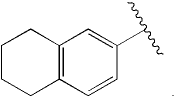

7) The compound of claim 1, wherein Ar₁ has one of following the formulas:

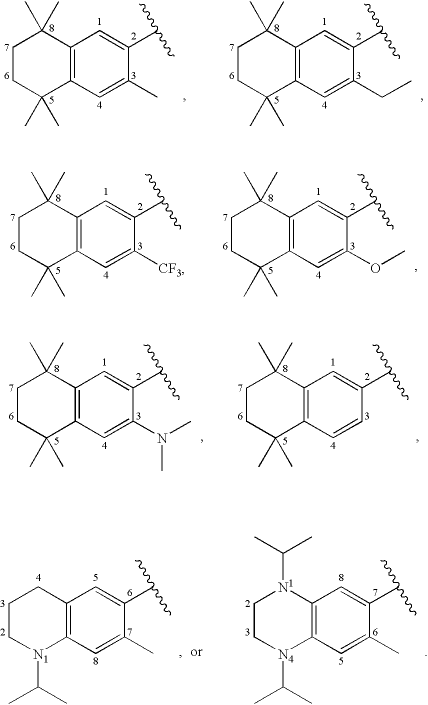

8) The compound of claim 1, wherein the Ar₂ aryl or heteroaryl ring has 0, 1, 2, or 3 non-hydrogen substituent groups, and Ar₂ and its substitutent groups together comprise between 4 and 20 carbon atoms.

9) The compound of claim 8, wherein the non-hydrogen substituent groups are independently selected from the group consisting of an alkyl, substituted alkyl, alkenyl, substituted alkenyl, alkynyl, substituted alkynyl, halogen, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, and alkylsulfonamide radical.

10) The compound of claim 1, wherein Ar₂ has one of the formulas: 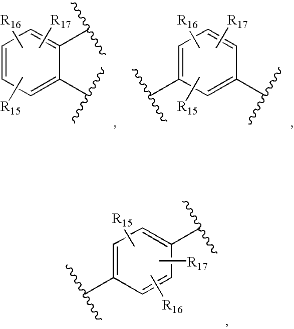
wherein R₁₅, R₁₆ and R₁₇ are independently selected from the group consisting of a hydrogen, alkyl, substituted alkyl, alkenyl, substituted alkenyl, alkynyl, substituted alkynyl, halogen, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, and alkylsulfonamide radical.

11) The compound of claim 1, wherein Ar₂ has one of the formulas; 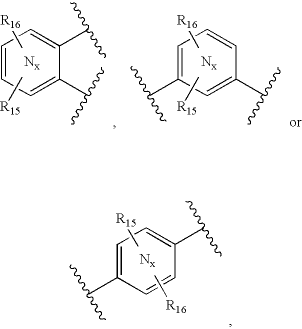
wherein Nₓ is 1 or 2 and the nitrogen atoms are unsubstituted ring atoms, R₁₅, R₁₆ are independently selected from the group consisting of a hydrogen, alkyl, substituted alkyl, alkenyl, substituted alkenyl, alkynyl, substituted alkynyl, halogen, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, and alkylsulfonamide radical.

12) The compound of claim 1, wherein Ar₂ has one of the formulas: 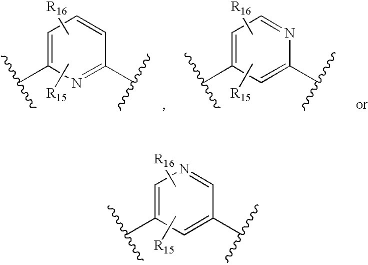
wherein R₁₅, and R₁₆ are independently selected from the group consisting of a hydrogen, alkyl, substituted alkyl, alkenyl, substituted alkenyl, alkynyl, substituted alkynyl, halogen, hydroxyl, acyloxy, alkoxy, substituted alkoxy, acyl, amino, mono-substituted amino, di-substituted amino, carboxy, carboalkoxy, alkylcarboxamide, substituted alkylcarboxamide, dialkylcarboxamide, substituted dialkylcarboxamide, and alkylsulfonamide radical.

13) The compound of claim 1, wherein R₁ is hydrogen, alkyl or substituted alkyl.

14) The compound of claim 1, wherein R₁ is hydrogen.

15) The compound of claim 1, wherein R₂ is hydrogen, alkyl or substituted alkyl.

16) The compound of claim 1, wherein R₁ and R₂ are hydrogen.

17) A compound having the formula:
3-(3,5,5,8,8-Pentamethyl-5,6,7,8-tetrahydro-2-naphthyl)-4-methoxybenzaldehyde oxime,
3-(3,5,5,8,8-Pentamethyl-5,6,7,8-tetrahydro-2-naphthyl)-4-trifluoromethoxybenzaldehyde oxime,
3-(3,5,5,8,8-Pentamethyl-5,6,7,8-tetrahydro-2-naphthyl)-4-dimethylaminobenzaldehyde oxime,
3-(3,5,5,8,8-Pentamethyl-5,6,7,8-tetrahydro-2-naphthyl)-2-fluoro-4-methoxybenzaldehyde oxime,
5-(3,5,5,8,8-Pentamethyl-5,6,7,8-tetrahydro-2-naphthyl)-6-methoxy-3-pyridinecarboxaldehyde oxime,
6-(3,5,5,8,8-Pentamethyl-5,6,7,8-tetrahydro-2-naphthyl)-5-methoxy-2-pyridinecarboxaldehyde oxime,
3-(3,5,5,8,8-Pentamethyl-5,6,7,8-tetrahydro-2-naphthyl)-4-methoxy-6-hydroxybenzaldehyde oxime,
3-(3,5,5,8,8-Pentamethyl-5,6,7,8-tetrahydro-2-naphthyl)-4,6-dimethoxybenzaldehyde oxime,
3-(3,5,5,8,8-Pentamethyl-5,6,7,8-tetrahydro-2-naphthyl)-4,6-dihydroxybenzaldehyde oxime,
3-(1,4-Diisopropyl-6-methyl-1,2,3,4-tetrahydro-7-quinoxalinyl)-4-methoxybenzaldehyde oxime, or
a pharmaceutically acceptable salt thereof.

18) A process for preparing a compound having the formula: 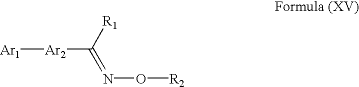
wherein:
a) Ar₁ comprises a substituted aryl or heteroaryl ring wherein two substituents together with the aryl or heteroaryl ring of Ar₁ together form an additional cycloalkyl, substituted cycloalkyl, cycloalkenyl or substituted cycloalkenyl ring radical optionally comprising 1 or 2 ring heteroatoms selected from O, S, SO, SO₂ and N, wherein N is further substituted with hydrogen, alkyl or substituted alkyl;
b) Ar₂ is a substituted or unsubstituted aryl radical or a substituted or unsubstituted heteroaryl radical;
c) R₁ is hydrogen, a substituted or unsubstituted amino radical, or a substituted or unsubstituted organic radical comprising from one to 12 carbon atoms; and
d) R₂ is hydrogen, or a substituted or unsubstituted organic radical comprising from one to 12 carbon atoms; comprising the steps of:
i) coupling an Ar₁ precursor compound with an Ar₂ precursor compound to give a biaryl carbonyl containing compound; wherein:
(1) the Ar₁ precursor compound has the structure: 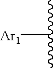
(2) and the Ar₂ precursor compound has a carbonyl group and has the structure: 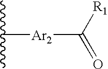
(3) and wherein the biaryl carbonyl containing compound has the structure: 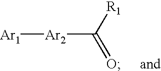
ii) condensing the biaryl carbonyl containing compound with a hydroxylamine derivative having the structure: 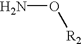
to give a compound of Formula (XV), or a pharmaceutically acceptable salt thereof.

19) The process of claim 18 wherein one of the Ar₁ or Ar₂ precursor compounds is an aryl boronic acid or ester, and the other Ar₁ or Ar₂ precursor compound is an aryl halide, triflate, or diazonium tetrafluoroborate.

20) The process of claim 18, wherein the coupling is conducted in the presence of a palladium catalyst.

21) A pharmaceutical composition comprising one or more compounds of claim 1 and a pharmaceutically acceptable carrier, for administration in mammals for modulating lipid metabolism, carbohydrate metabolism, lipid and carbohydrate metabolism, or adipocyte differentiation.

22) A pharmaceutical composition of claim 21 wherein the administration treats type 2 diabetes, polycystic ovary syndrome or syndrome X.

23) A method of modulating lipid metabolism, carbohydrate metabolism, lipid and carbohydrate metabolism, or adipocyte differentiation in a mammal, comprising administering the pharmaceutical composition of claim 21 to a mammal in an amount that is effective to change the rate of lipid or carbohydrate metabolism, or change the rate of adipocyte differentiation, as compared to the rate of lipid or carbohydrate metabolism, or the rate of adipocyte differentiation that occurs in the absence of the pharmaceutical composition.

24) The method of claim 23 wherein the mammal is a human.

25) A method of treating type 2 diabetes comprising administering to a mammal diagnosed as having type 2 diabetes an amount of the pharmaceutical composition of claim 21 that is effective to treat the type 2 diabetes.

26) The method of claim 25 wherein the mammal is a human.
